(19) United States
(12) Patent Application Publication
(10) Pub. No.: US 2025/0325711 A1
(43) Pub. Date: Oct. 23, 2025

(54) COVALENT FLUORESCENT PROBES FOR CANNABINOID RECEPTOR 2

(71) Applicant: Hoffmann-La Roche Inc., Little Falls, NJ (US)

(72) Inventors: Miroslav Kosar, Urdorf (CH); Erick Moran Carreira, Zuerich (CH); Uwe Grether, Efringen-Kirchen (DE); Roman Clà Sarott, Zuerich (CH); Wolfgang Guba, Müllheim (DE); Rudolf Liun Zaccaria Ganzoni, Zürich (CH)

(73) Assignee: Hoffmann-La Roche Inc., Little Falls, NJ (US)

(21) Appl. No.: 19/064,442

(22) Filed: Feb. 26, 2025

Related U.S. Application Data
(63) Continuation of application No. PCT/EP2023/073188, filed on Aug. 24, 2023.

(30) Foreign Application Priority Data
Aug. 26, 2022 (EP) 22192302.2

(51) Int. Cl. A61K 49/00; C07C 307/06; C07D 271/12; C07D 293/10; C07D 401/14; C07D 405/14; C07D 413/12; C07D 413/14; C07F 7/08

(52) U.S. Cl. CPC A61K 49/0052; C07C 307/06; C07D 271/12; C07D 293/10; C07D 401/14; C07D 405/14; C07D 413/12; C07D 413/14; C07F 7/0812; A61K 2123/00

(57) ABSTRACT

The present invention provides covalent fluorescent probes for cannabinoid receptor 2 (“CB2”) having the general formula (I)

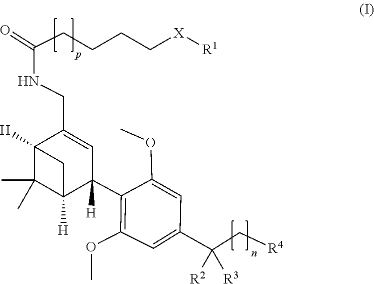

wherein X, n, p, and R¹ to R⁴ are as described herein, compositions including the compounds, processes of manufacturing the compounds and methods of using the compounds.

### CROSS-REFERENCE TO RELATED APPLICATIONS

This application is a continuation of PCT Application No. PCT/EP2023/073188, filed Aug. 24, 2023, which claims benefit of priority to EP Application No. 22192302.2, filed Aug. 26, 2022, each of which is incorporated herein by reference in its entirety.

### FIELD OF THE INVENTION

The present invention relates to organic compounds useful as affinity-driven covalent probes for the cannabinoid receptor 2 (CB₂R). The probes are achieved by a novel, convergent synthetic blueprint from a central reactive motif.

### BACKGROUND OF THE INVENTION

Dysregulation of the endocannabinoid system, specifically of signalling pathways encompassed by CB₂R, has been implicated in a range of diseases including tissue injury, neurodegenerative conditions and inflammation. Despite the evident importance of CB₂R no compounds have been brought to the clinic. This feature is primarily ascribed to insufficient understanding of CB₂R biology, particularly expression and localization in health and disease states. Fluorescent imaging probes have recently emerged as high-resolution tools to allow investigation of CB₂R localization, expression levels and distribution in living cells and in vivo (R. C. Sarott, et. al., J Am. Chem. Soc., 2020, 142, 16953-16964, T. Gazzi, et. al., Chem. Sci. 2022, 13, 5539-5545). However, these reported probes are standard affinity fluorescent probes that bind the orthosteric site of CB₂R and with their strong agonist ligands trigger associated signalling pathways and finally result in desensitization by receptor internalization. Even in the case of antagonist or inverse agonist CB₂R probes (S. Singh, et. al., ACS Med. Chem. Lett. 2019, 10, 209-214) that do not inherently trigger signalling, the probes modulate the cellular homeostasis as they constantly occupy the orthosteric site of CB₂R and hence directly change the cellular tone by preventing the binding of endogenous ligands. As such, there is currently no available probe to study CB₂R without concurrent change in the receptors signalling pathways, localization, expression, trafficking and/or distribution.

### SUMMARY OF THE INVENTION

The limitations outlined above are addressed by this invention which reports CB₂R affinity-driven probes that carry a cleavable motif which allows covalent transfer of a reporter unit to study the target protein with concomitant release of the targeting ligand (T. Tamura, I. 5 Hamachi, J. Am. Chem. Soc. 2019, 141, 2782-2799). Such probes can be applied in flow cytometry fluorescence-activated cell sorting (FACS) experiments or cellular studies using confocal live cell imaging, for example, to quantify trafficking of low abundance targets. In addition, such probes offer the potential for generating equilibrium and kinetic binding data in a high-throughput fashion, without handling radioactive material using, for example, time-resolved Forster resonance energy transfer (TR-FRET). Furthermore, the probes are especially well suited to construct FRET biosensors on the surface and inside live cells to study ligand-endogenous protein binding interactions in real time (K. Matsuo, et. al., Angew. Chem. Int. Ed. 2018, 57, 659-662). Due to the covalent nature of reporter transfer, such probes are ideally suited to target a low expression target such as CB₂R in a healthy brain. The probes can also support the translation of preclinical pharmacological animal data to clinics.

In a first aspect, the present invention provides a compound of Formula (I)

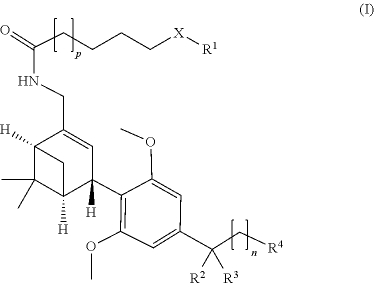

or pharmaceutically acceptable salts thereof, wherein X, n, p, and R¹ to R⁴ are as defined herein.

In a further aspect, the present invention provides processes for manufacturing fluorescent and bioorthogonal probes for proteins of interest, including the compounds of formula (I) as described herein.

In a further aspect, the present invention provides a process that enables orthogonal formation of amides in the presence of labile, highly electrophilic functional groups.

In a further aspect, the present invention provides certain synthetic intermediates that are useful in the processes according to the invention.

In a further aspect, the present invention provides the use of a compound of formula (I) described herein as a fluorescent probe, or a probe for secondary bioorthogonal conjugation, for the cannabinoid receptor 2 (CB₂R).

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms, e.g., 1, 2, 3, 4, 5, or 6 carbon atoms (“C₁-C₆-alkyl”). In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “cycloalkyl” as used herein refers to a saturated or partly unsaturated monocyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. Preferably, the cycloalkyl group is a saturated monocyclic hydrocarbon group of 3 to 6 ring carbon atoms, in particular of 3 to 5 ring carbon atoms, e.g., of 3, 4 or 5 carbon atoms. Some non-limiting examples of cycloalkyl include cyclopropyl, cyclobutyl, cyclopentyl, cyclohexyl and cycloheptyl. A preferred, yet non-limiting example of cycloalkyl is cyclopropyl.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cyano” refers to a —CN (nitrile) group.

The term “azide” refers to a —N₃ group.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride and trifluoroacetatesalts.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “CB2” refers to the cannabinoid receptor 2.

### Compounds of the Invention

In a first aspect (A1), the present invention provide a compound of Formula (I)

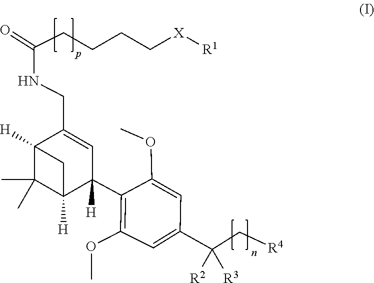

or a pharmaceutically acceptable salt thereof, wherein.
(i) R¹ is

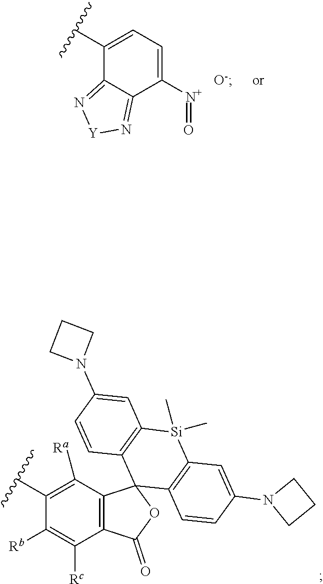

and
   X is selected from O, S, and SO; or
(ii) R¹ is

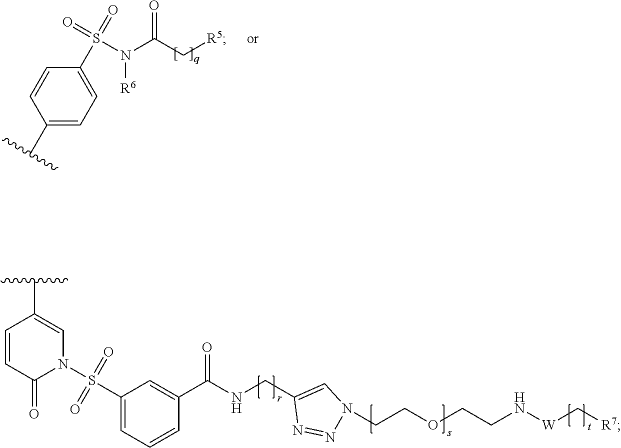

and
   X is selected from O, S, SO, and NHCO;
R² and R³ are selected from hydrogen and C₁-C₄-alkyl; or
R² and R³, taken together with the carbon atom to which they are attached, form a C₃-C₅-cycloalkyl;
R⁴ is selected from hydrogen, halogen, cyano, and azide;
R⁵ is selected from:

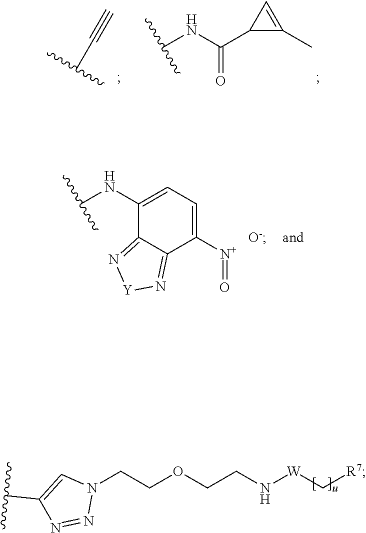

R⁶ is selected from:

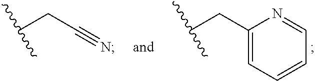

R⁷ is selected from:

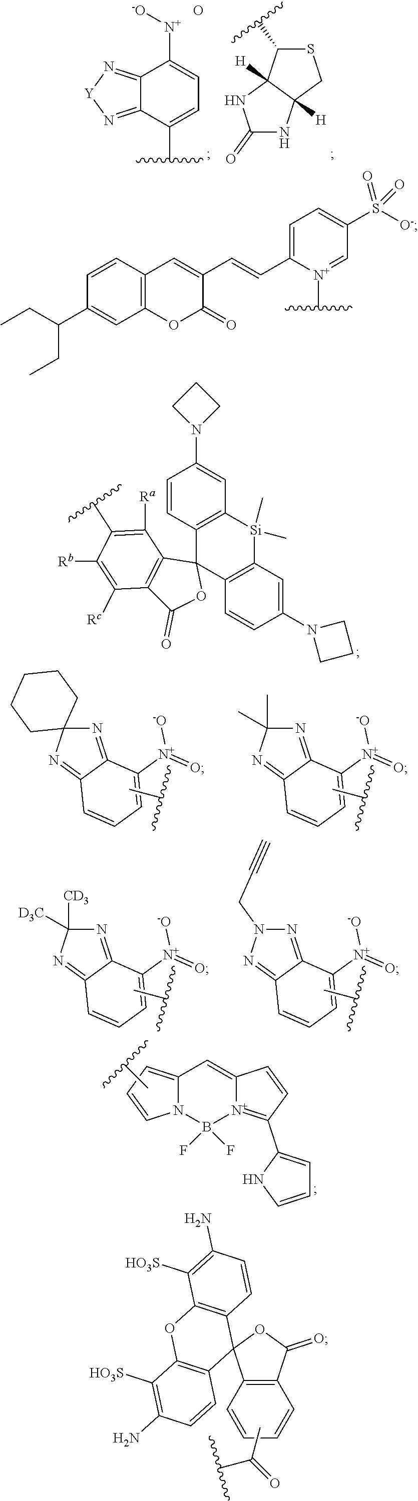

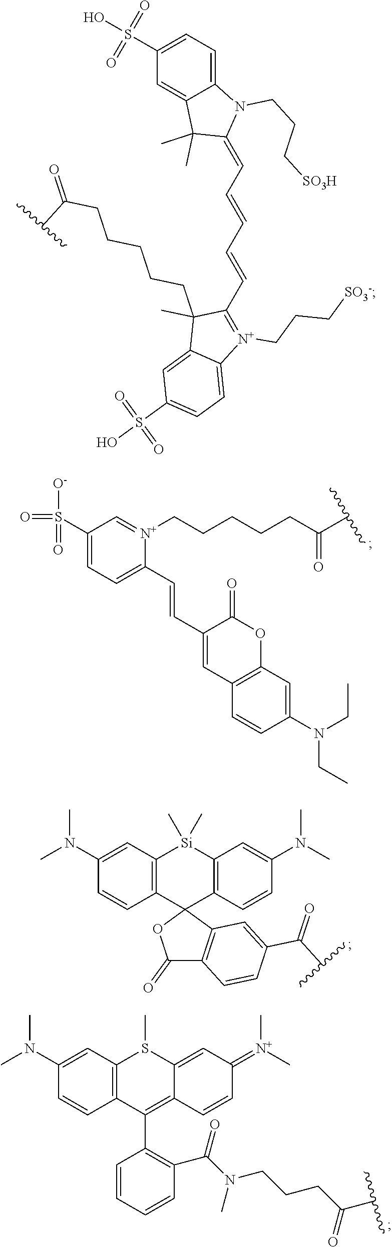

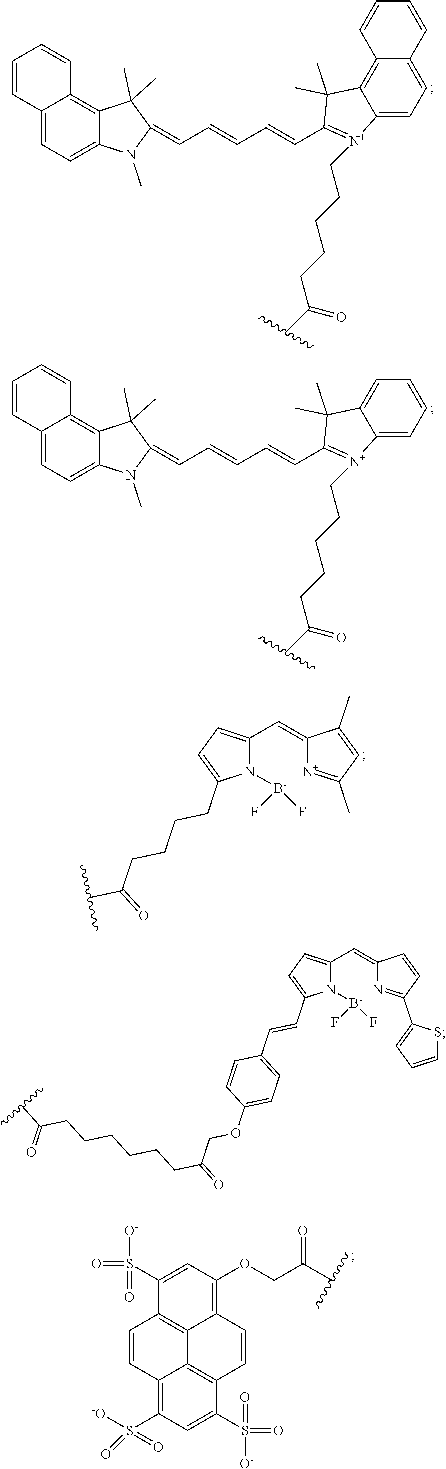

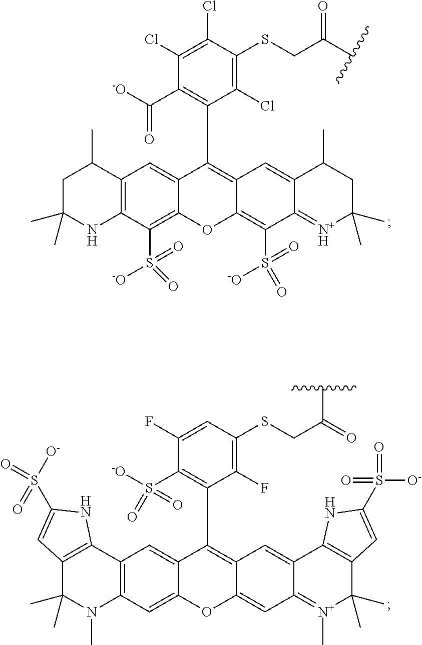

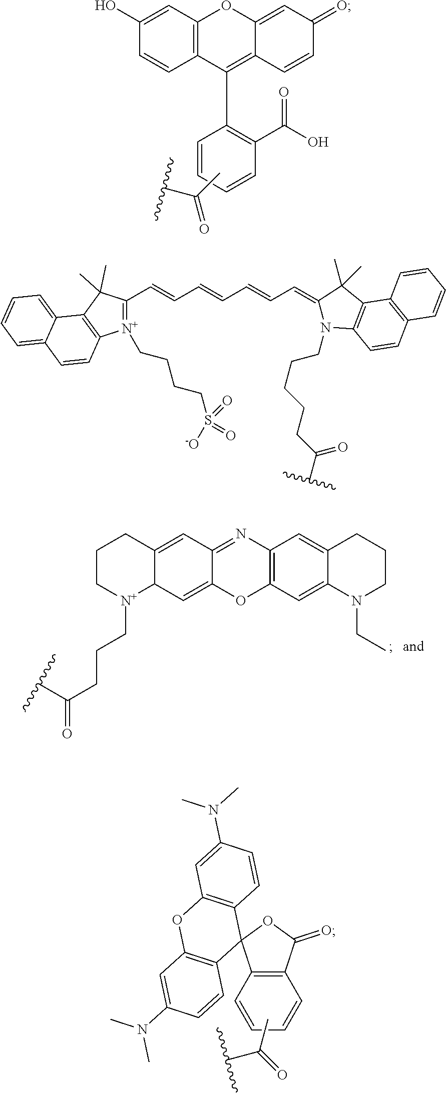

R^{a}, R^{b}, and R^{c} are each independently selected from hydrogen and halogen;
n is selected from 1, 2, 3, 4, 5 and 6;
p is selected from 1, 2, 3, 4, 5, 6, and 7;
q is selected from 1, 2, 3, 4, and 5;
r is selected from 1 and 2;
s is selected from 1, 2, 3, and 4;
t is selected from 0, 1, 2, 3, 4, and 5;
u is selected from 0, 1, 2, 3, 4, and 5;
W is carbonyl or absent;
Y is selected from Se, S, and O.

In one embodiment, the present invention provides a compound of formula (I) as described herein, or a pharmaceutically acceptable salt thereof, wherein:
(i) R¹ is

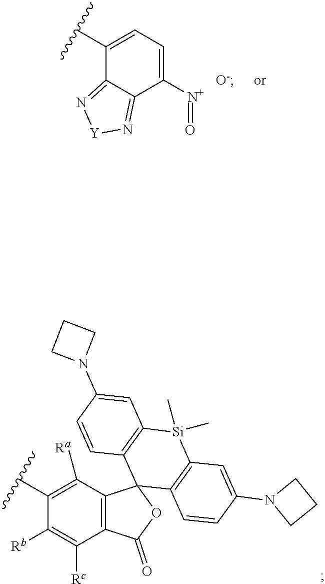

and
   X is selected from O, S, and SO; or
(ii) R¹ is

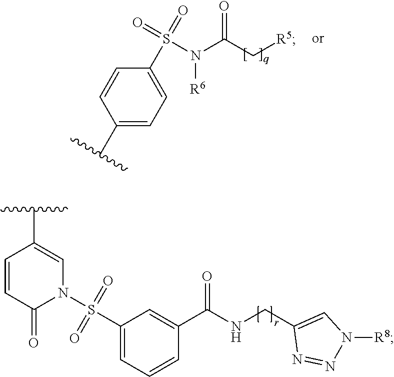

and
   X is selected from O, S, SO, and NHCO;
R² and R³ are selected from hydrogen and C₁-C₄-alkyl; or
R² and R³, taken together with the carbon atom to which they are attached, form a C₃-C₅-cycloalkyl;
R⁴ is selected from hydrogen, halogen, cyano, and azide;
R⁵ is selected from:

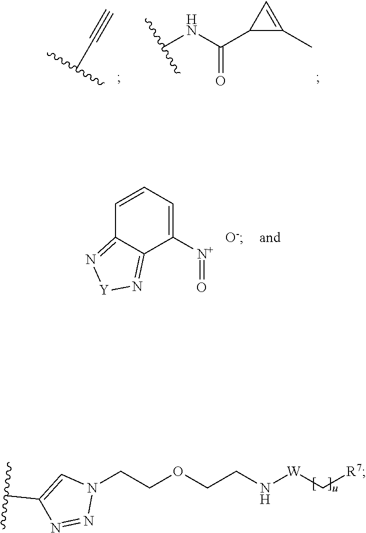

R⁶ is selected from:

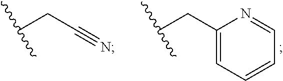

R⁷ is selected from:

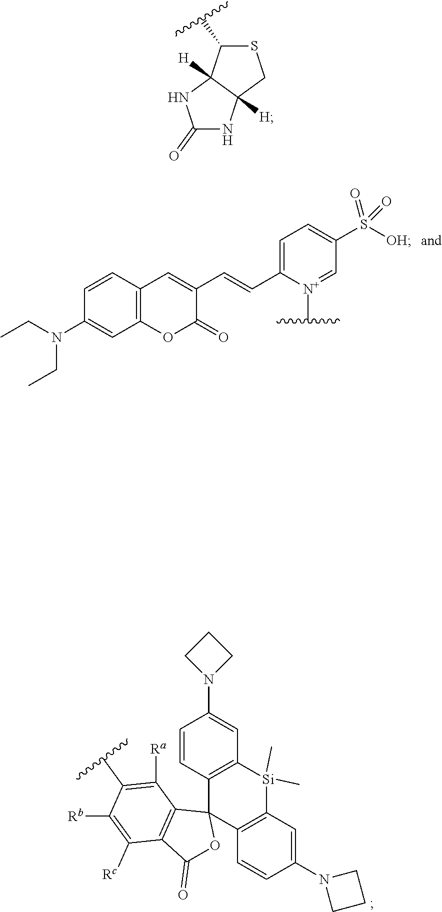

R⁸ is selected from:

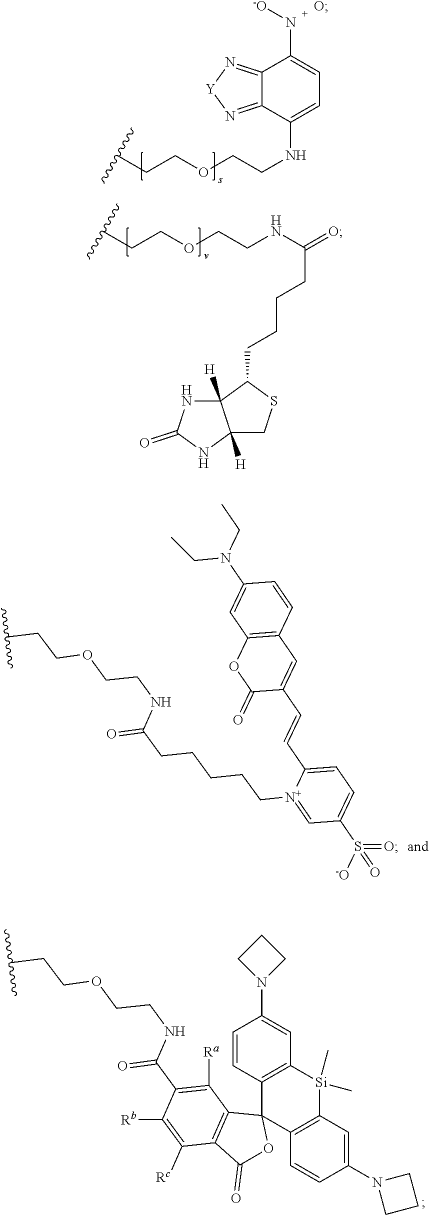

R^{a}, R^{b}, and R^{c} are each independently selected from hydrogen and halogen;
n is selected from 1, 2, 3, 4, 5 and 6;
p is selected from 1, 2, 3, 4, 5, 6, and 7;
q is selected from 1, 2, 3, 4, and 5;
r is selected from 1 and 2;
s is selected from 1, 2, 3, and 4;
u is selected from 0, 1, 2, 3, 4, and 5;
v is selected from 1, 2, 3, and 4;
W is carbonyl or absent;
Y is selected from Se, S, and O.

In one embodiment, the present invention provides a compound of formula (I) as described herein, or a pharmaceutically acceptable salt thereof, wherein:
(i) R¹ is

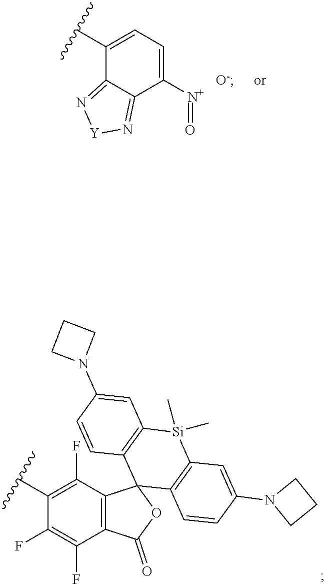

and
   X is selected from O, S, and SO; or
(ii) R¹ is

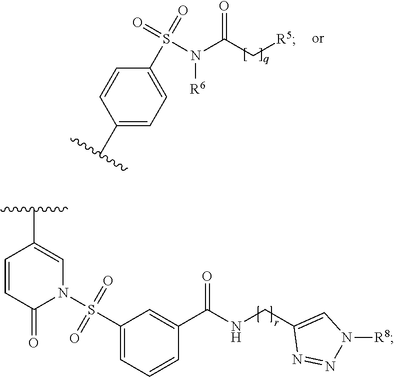

and
   X is NHCO;
R⁵ is selected from:

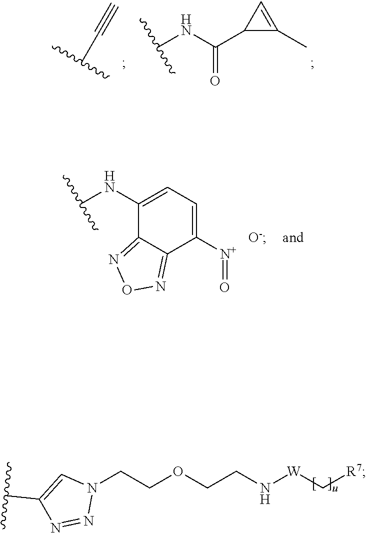

R⁶ is selected from:

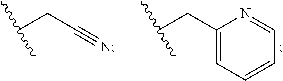

R⁷ is selected from:

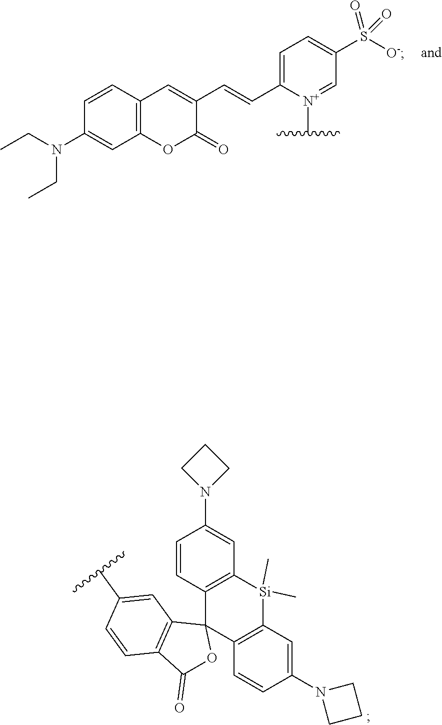

R⁸ is selected from:

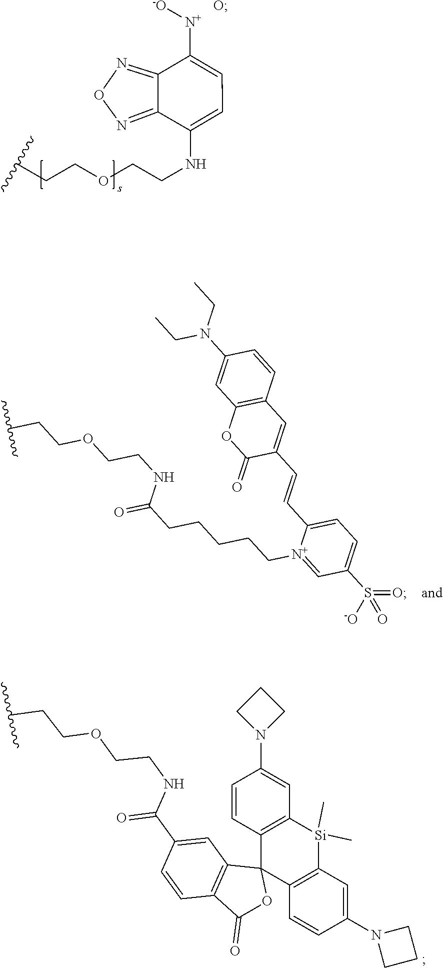

p is selected from 3, 4, and 5;
q is selected from 2, 3, and 5;
r is selected from 1 and 2;
s is selected from 1 and 4;
u is selected from 0, 4, and 5; and
W is carbonyl or absent;
Y is selected from Se and O.

In a preferred embodiment, the present invention provides a compound of formula (I) as described herein, or a pharmaceutically acceptable salt thereof, wherein:
(1) R¹ is

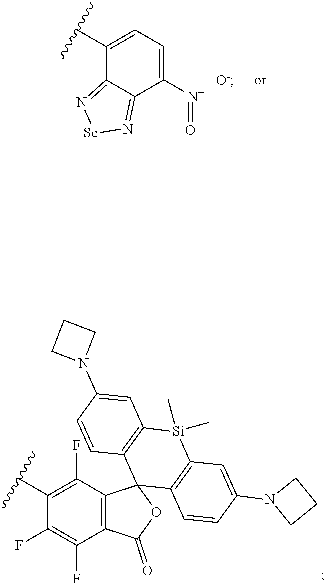

and
   X is O; or
(ii) R¹ is

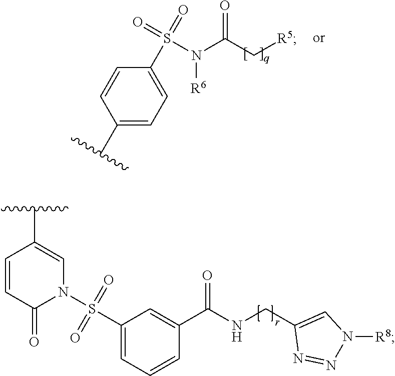

and
   X is NHCO;
R⁵ is selected from:

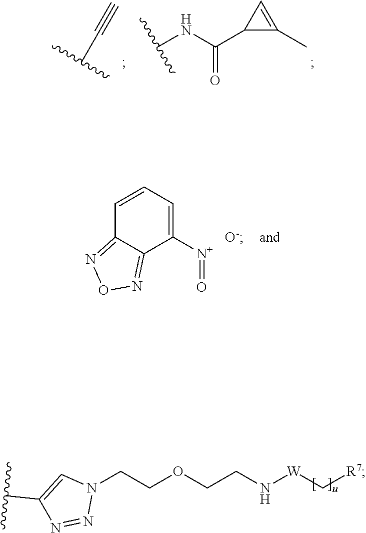

R⁶ is selected from:

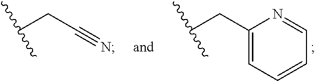

R⁷ is selected from:

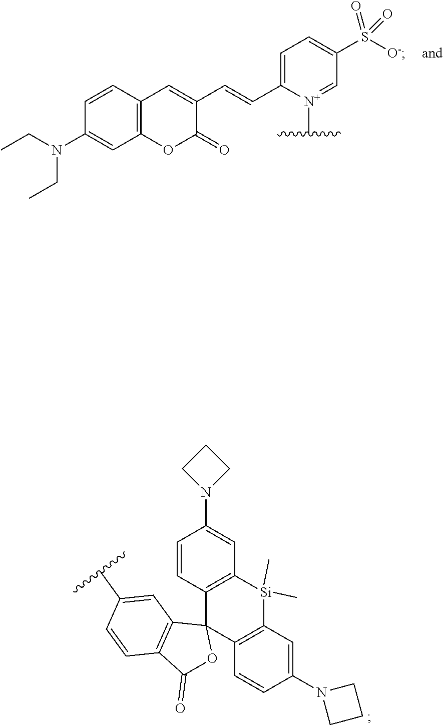

R⁸ is selected from:

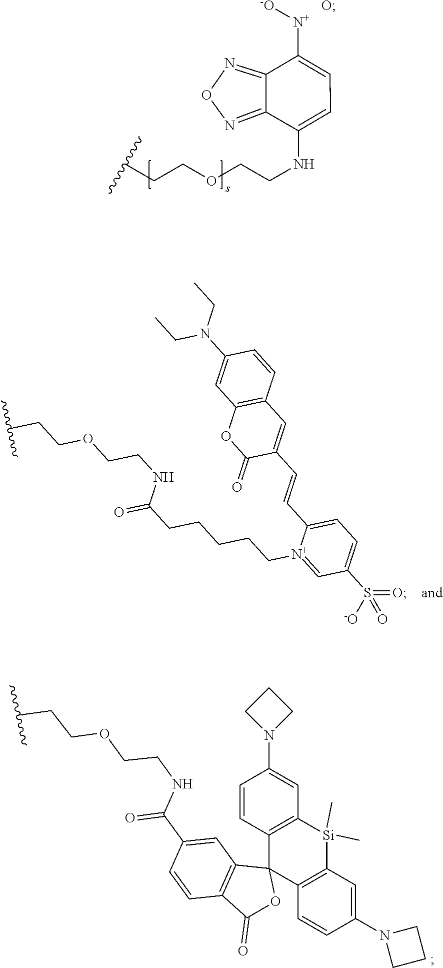

p is 3;
q is selected from 2, 3, and 5;
r is 1;
s is 1; and
u is 5.

In a particularly preferred embodiment, the present invention provides a compound of formula (I) as described herein, or a pharmaceutically acceptable salt thereof, wherein:
R¹ is

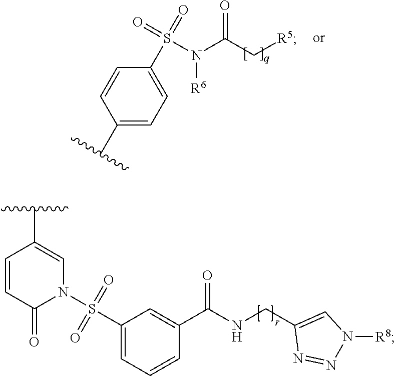

R⁵ is

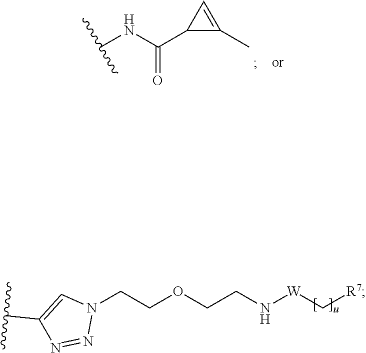

R⁶ is

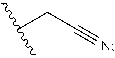 [SMILES: *CC#N]

R⁷ is

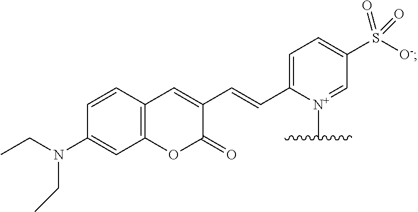

R⁸ is

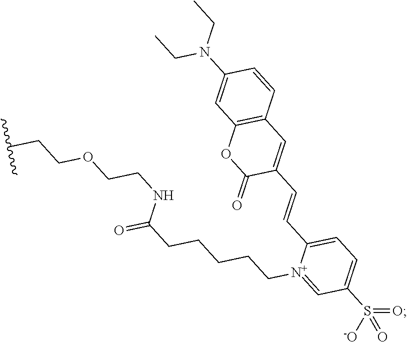

X is NHCO;
p is 3;
q is 2 or 3;
r is 1; and
u is 5.

In one embodiment, the present invention provides a compound of formula (I) as described herein, or a pharmaceutically acceptable salt thereof, wherein:
R¹ is

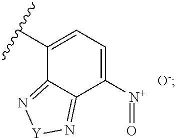

and
X is selected from O, S, and SO.

In one embodiment, the present invention provides a compound of formula (I) as described herein, or a pharmaceutically acceptable salt thereof, wherein:
R¹ is

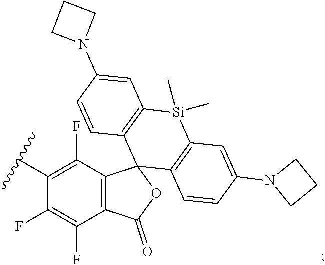 [SMILES: *c1c(F)c(F)c2c(c1F)C1(OC2=O)c2ccc(N3CCC3)cc2[Si](C)(C)c2cc(N3CCC3)ccc21]

and
X is selected from O, S, and SO.

In one embodiment, the present invention provides a compound of formula (I) as described herein, or a pharmaceutically acceptable salt thereof, wherein:
R¹ is

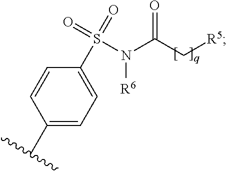

and
X is NHCO; wherein
R⁵, R⁶, and q are as defined herein.

In one embodiment, the present invention provides a compound of formula (I) as described herein, or a pharmaceutically acceptable salt thereof, wherein:
R¹ is

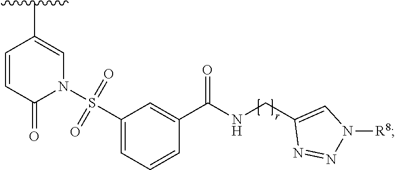

and
X is NHCO; wherein
R⁸ and r are as defined herein.

In a preferred embodiment, the present invention provides a compound of formula (I) as described herein, or a pharmaceutically acceptable salt thereof, wherein:
R¹ is

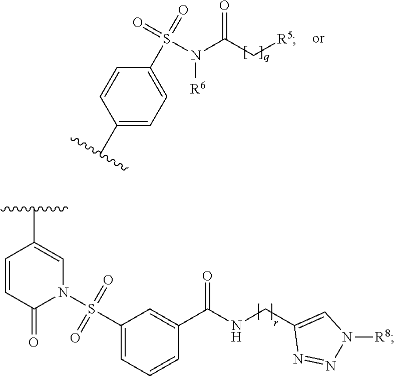

and
X is NHCO; wherein
R⁵, R⁶, R⁸, q and r are as defined herein.

In one embodiment, the present invention provides a compound of formula (I) as described herein, or a pharmaceutically acceptable salt thereof, wherein:
R⁵ is selected from:

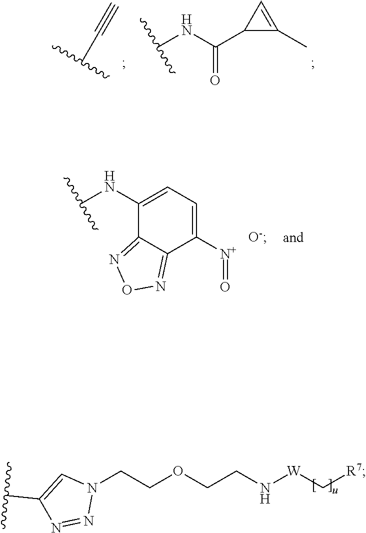

wherein
R⁷, W, and u are as defined herein.

In a preferred embodiment, the present invention provides a compound of formula (I) as described herein, or a pharmaceutically acceptable salt thereof, wherein:
R⁵ is

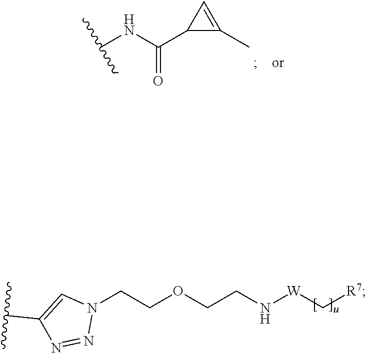

wherein
R⁷ and u are as defined herein.

In one embodiment, the present invention provides a compound of formula (I) as described herein, or a pharmaceutically acceptable salt thereof, wherein:
R⁶ is

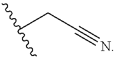 [SMILES: *CC#N]

In one embodiment, the present invention provides a compound of formula (I) as described herein, or a pharmaceutically acceptable salt thereof, wherein:
R⁶ is

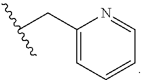 [SMILES: *Cc1ccccn1]

In one embodiment, the present invention provides a compound of formula (I) as described herein, or a pharmaceutically acceptable salt thereof, wherein:
R⁷ is selected from:

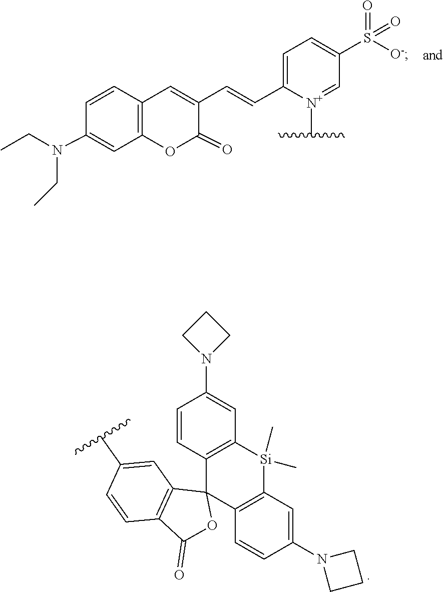

In a particularly preferred embodiment, the present invention provides a compound of formula (I) as described herein, or a pharmaceutically acceptable salt thereof, wherein:
R⁷ is

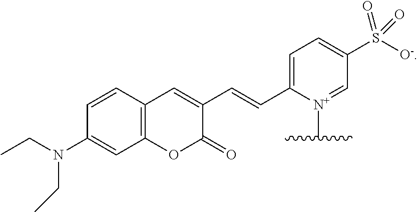

In a preferred embodiment, the present invention provides a compound of formula (I) as described herein, or a pharmaceutically acceptable salt thereof, wherein:
R⁷ is

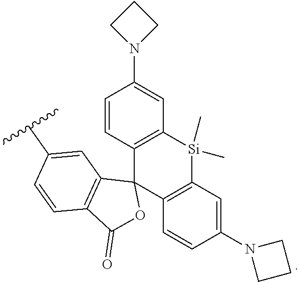 [SMILES: *c1ccc2c(c1)C1(OC2=O)c2ccc(N3CCC3)cc2[Si](C)(C)c2cc(N3CCC3)ccc21]

In one embodiment, the present invention provides a compound of formula (I) as described herein, or a pharmaceutically acceptable salt thereof, wherein:
R⁸ is selected from:

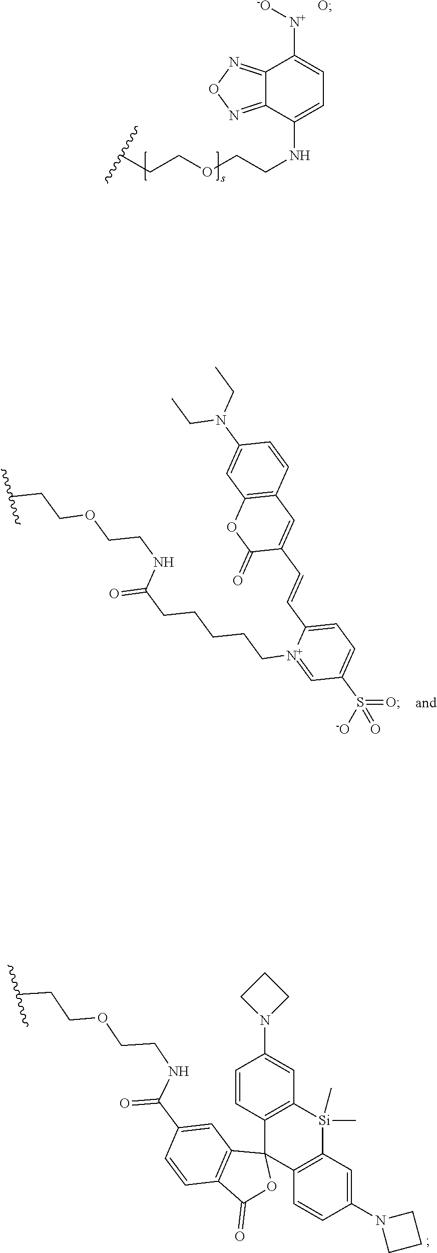

wherein
s is as defined herein.

In a preferred embodiment, the present invention provides a compound of formula (I) as described herein, or a pharmaceutically acceptable salt thereof, wherein:
R⁸ is

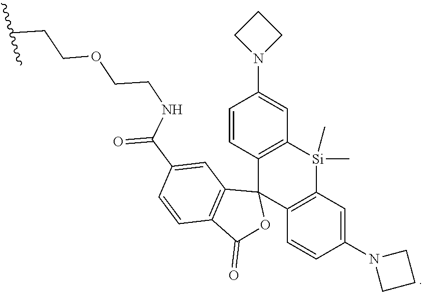 [SMILES: *CCOCCNC(=O)c1ccc2c(c1)C1(OC2=O)c2ccc(N3CCC3)cc2[Si](C)(C)c2cc(N3CCC3)ccc21]

In a preferred embodiment, the present invention provides a compound of formula (I) as described herein, or a pharmaceutically acceptable salt thereof, wherein:
R⁸ is

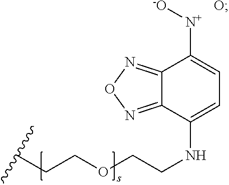

wherein
s is as defined herein.

In a particularly preferred embodiment, the present invention provides a compound of formula (I) as described herein, or a pharmaceutically acceptable salt thereof, wherein:
R⁸ is

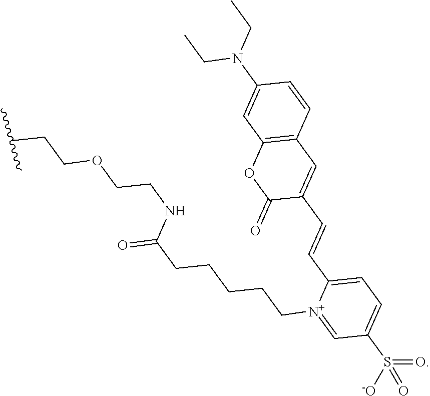

In one embodiment, the present invention provides a compound of formula (I) as described herein, or a pharmaceutically acceptable salt thereof, wherein:
p is selected from 1, 3, 4, and 5.

In a preferred embodiment, the present invention provides a compound of formula (I) as described herein, or a pharmaceutically acceptable salt thereof, wherein:
p is 1.

In a particularly preferred embodiment, the present invention provides a compound of formula (I) as described herein, or a pharmaceutically acceptable salt thereof, wherein:
p is 3.

In a preferred embodiment, the present invention provides a compound of formula (I) as described herein, or a pharmaceutically acceptable salt thereof, wherein:
p is 4.

In a preferred embodiment, the present invention provides a compound of formula (I) as described herein, or a pharmaceutically acceptable salt thereof, wherein:
p is 5.

In one embodiment, the present invention provides a compound of formula (I) as described herein, or a pharmaceutically acceptable salt thereof, wherein:
q is selected from 2, 3, and 5.

In a preferred embodiment, the present invention provides a compound of formula (I) as described herein, or a pharmaceutically acceptable salt thereof, wherein:
q is 2 or 3.

In one embodiment, the present invention provides a compound of formula (I) as described herein, or a pharmaceutically acceptable salt thereof, wherein:
r is selected from 1 and 2.

In a preferred embodiment, the present invention provides a compound of formula (I) as described herein, or a pharmaceutically acceptable salt thereof, wherein:
r is 1.

In one embodiment, the present invention provides a compound of formula (I) as described herein, or a pharmaceutically acceptable salt thereof, wherein:
s is selected from 1 and 4.

In one embodiment, the present invention provides a compound of formula (I) as described herein, or a pharmaceutically acceptable salt thereof, wherein:
u is selected from 0, 4, and 5.

In a preferred embodiment, the present invention provides a compound of formula (I) as described herein, or a pharmaceutically acceptable salt thereof, wherein:
u is 5.

In one embodiment, the present invention provides a compound of formula (I) as described herein, or a pharmaceutically acceptable salt thereof, wherein:
Y is selected from Se and O.

In a preferred embodiment, the present invention provides a compound of formula (I) as described herein, or a pharmaceutically acceptable salt thereof, wherein:
s is 1.

In a preferred embodiment, the present invention provides a compound of formula (I) as described herein, or a pharmaceutically acceptable salt thereof, wherein:
u is 5.

In one embodiment, the present invention provides a compound of formula (I) as described herein, or a pharmaceutically acceptable salt thereof, wherein:
R² and R³ are selected from hydrogen and C₁-C₄-alkyl;
R⁴ is hydrogen or azide; and
n is 6.

In one embodiment, the present invention provides a compound of formula (I) as described herein, or a pharmaceutically acceptable salt thereof, wherein:
R² and R³ are both C₁-C₄-alkyl;
R⁴ is azide; and
n is 6.

In one embodiment, the present invention provides a compound of formula (I) as described herein, or a pharmaceutically acceptable salt thereof, wherein:
R² and R³ are both methyl;
R⁴ is azide; and
n is 6.

In one embodiment, the present invention provides a compound of formula (I) as described herein, or a pharmaceutically acceptable salt thereof, wherein:
R² and R³ are selected from hydrogen and C₁-C₄-alkyl.

In a preferred embodiment, the present invention provides a compound of formula (I) as described herein, or a pharmaceutically acceptable salt thereof, wherein:
R² and R³ are both C₁-C₄-alkyl.

In one embodiment, the present invention provides a compound of formula (I) as described herein, or a pharmaceutically acceptable salt thereof, wherein:
R² and R³ are both hydrogen.

In a particularly preferred embodiment, the present invention provides a compound of formula (I) as described herein, or a pharmaceutically acceptable salt thereof, wherein:
R² and R³ are both methyl.

In one embodiment, the present invention provides a compound of formula (I) as described herein, or a pharmaceutically acceptable salt thereof, wherein:
R⁴ is hydrogen or azide.

In one embodiment, the present invention provides a compound of formula (I) as described herein, or a pharmaceutically acceptable salt thereof, wherein:
R⁴ is hydrogen.

In a preferred embodiment, the present invention provides a compound of formula (I) as described herein, or a pharmaceutically acceptable salt thereof, wherein:
R⁴ is azide.

In one embodiment, the present invention provides a compound of formula (I) as described herein, or a pharmaceutically acceptable salt thereof, wherein:
n is 6.

In one embodiment, the present invention provides a compound of formula (I) as described herein, or a pharmaceutically acceptable salt thereof, wherein:
(i) R¹ is

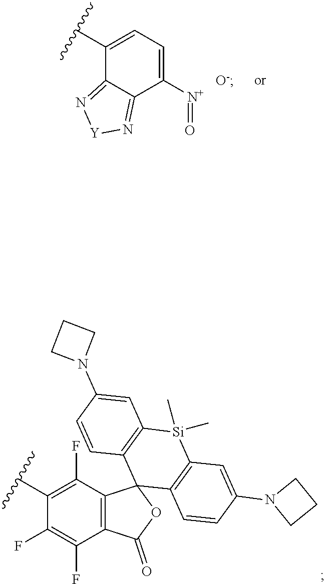

and
   X is selected from O, S, and SO; or
(ii) R¹ is

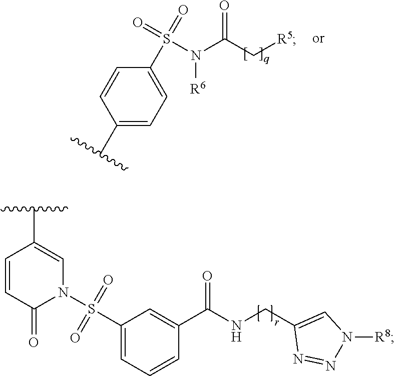

and
   X is NHCO;
R² and R³ are selected from hydrogen and C₁-C₄-alkyl;
R⁴ is hydrogen or azide;
R⁵ is selected from:

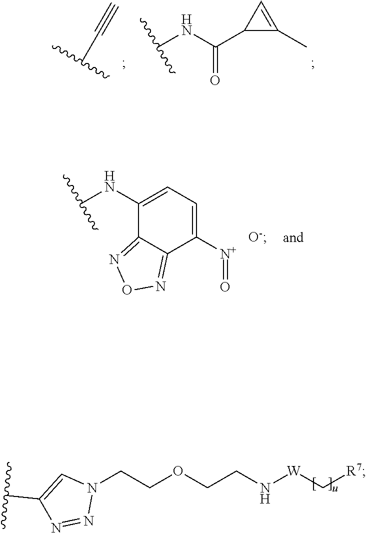

R⁶ is selected from:

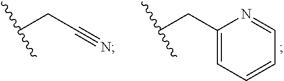

R⁷ is selected from:

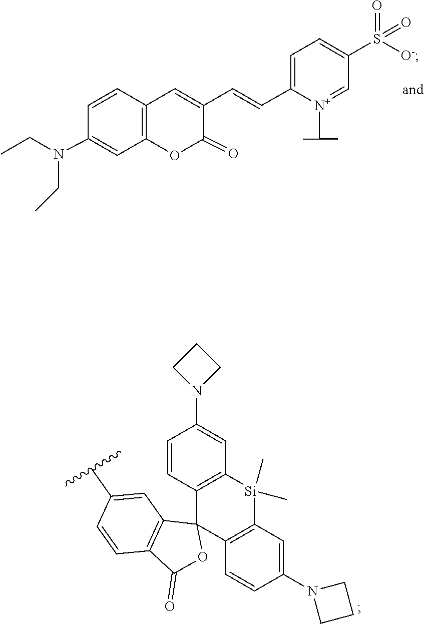

R⁸ is selected from:

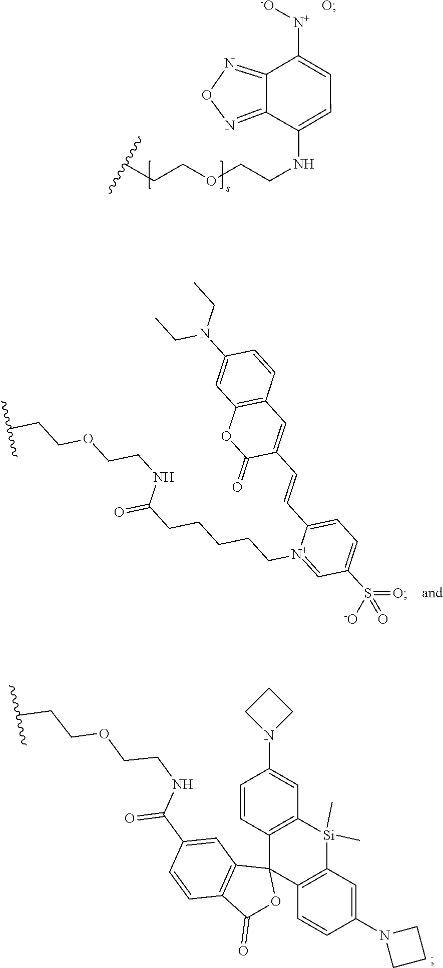

n is 6;
p is selected from 1, 3, 4, and 5;
q is selected from 2, 3, and 5;
r is selected from 1 and 2;
s is selected from 1 and 4;
u is selected from 0, 4, and 5;
W is carbonyl or absent;
Y is selected from Se and O.

In a preferred embodiment, the present invention provides a compound of formula (I) as described herein, or a pharmaceutically acceptable salt thereof, wherein:
(i) R¹ is

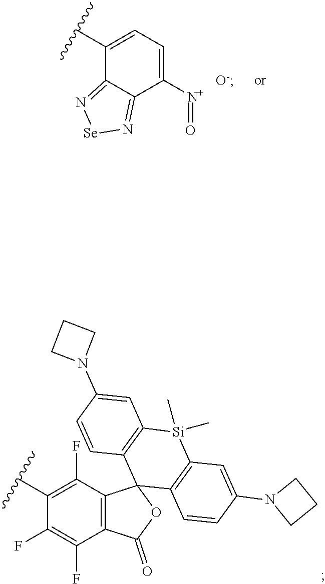

and
   X is O; or
(ii) R¹ is

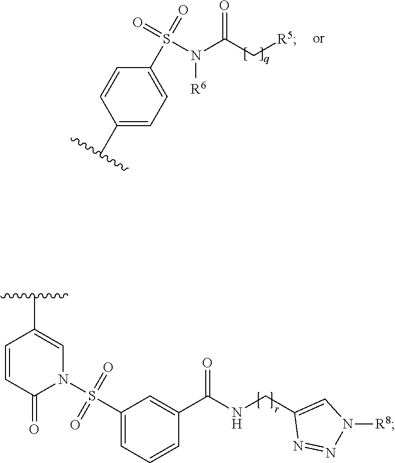

and
   X is NHCO;
R² and R³ are both C₁-C₄-alkyl;
R⁴ is azide;
R⁵ is selected from:

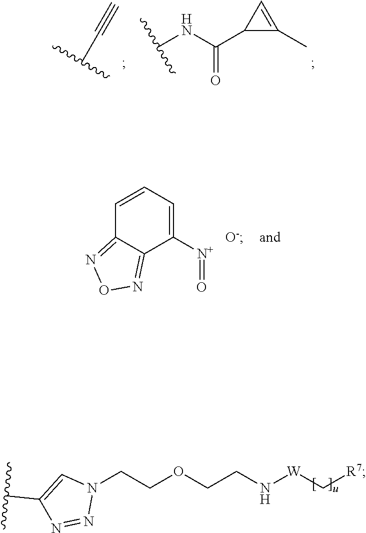

R⁶ is selected from:

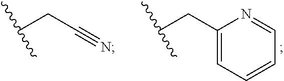

R⁷ is selected from:

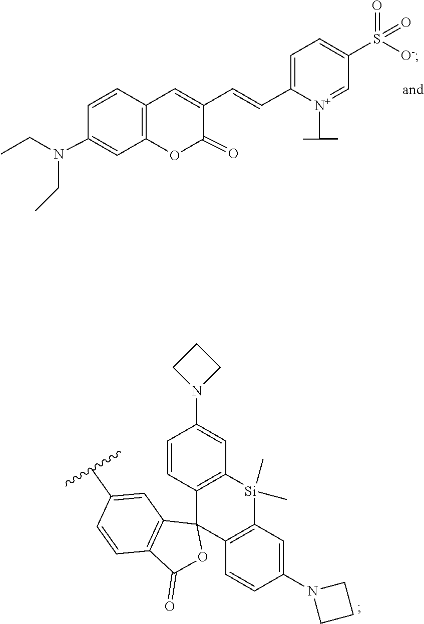

R⁸ is selected from:

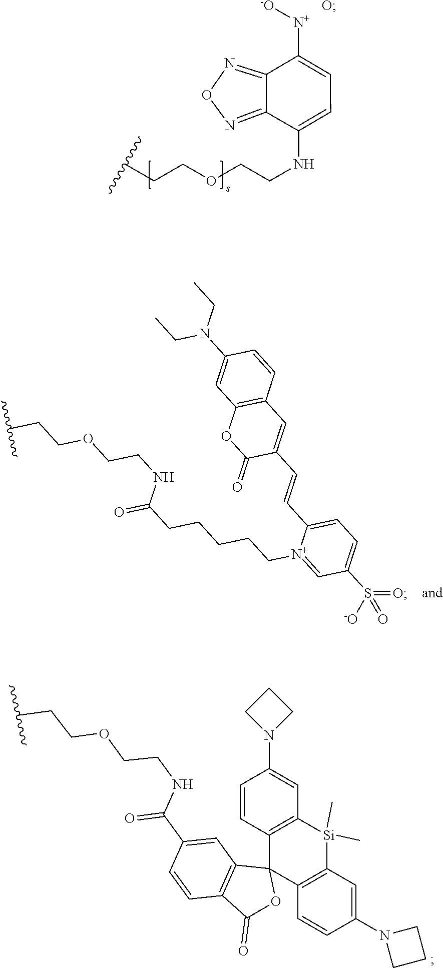

n is 6;
p is 3;
q is selected from 2, 3, and 5;
r is 1;
s is 1; and
u is 5.

In a particularly preferred embodiment, the present invention provides a compound of formula (I) as described herein, or a pharmaceutically acceptable salt thereof, wherein:
R¹ is

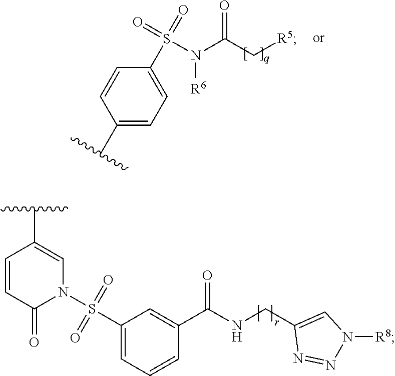

R² and R³ are both methyl;
R⁴ is azide;
R⁵ is

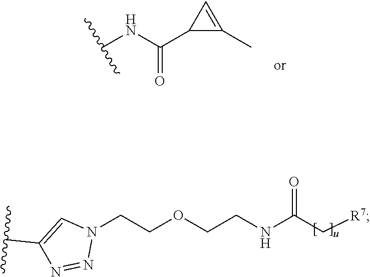

R⁶ is

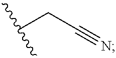 [SMILES: *CC#N]

R⁷ is

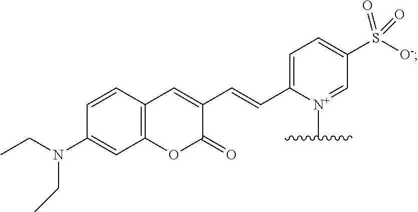

R⁸ is

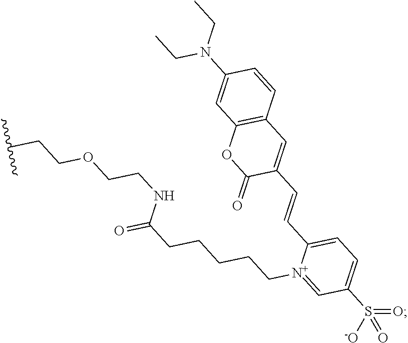

X is NHCO;
n is 6;
p is 3;
q is 2 or 3;
r is 1; and
u is 5.

In one embodiment, the present invention provides a compound of formula (I) as described herein, or a pharmaceutically acceptable salt thereof, wherein the compound of formula (I) is selected from:
N-(9-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-9-oxononyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(5-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-5-oxopentyl)-1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamide;
1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(6-((7-nitrobenzo[c][1,2,5]oxadiazol-4-yl)amino)hexanoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamide;
1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate;
3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)thio)octanamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)sulfinyl)octanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-(5-((3aS,4S,6aR)-2-oxohexahydro-1H-thieno[3,4-d]imidazol-4-yl)pentanamido)ethoxy)ethyl)-1H-1,2,3-triazol-4-yl)propanoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(16-oxo-20-((3aS,4S,6aR)-2-oxohexahydro-1H-thieno[3,4-d]imidazol-4-yl)-3,6,9,12-tetraoxa-15-azaicosyl)-1H-1,2,3-triazol-4-yl)propanoyl)sulfamoyl)benzamide; and
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-6-oxo-1-((3-(((1-(2-(2-(5-((3aS,4S,6aR)-2-oxohexahydro-1H-thieno[3,4-d]imidazol-4-yl)pentanamido)ethoxy)ethyl)-1H-1,2,3-triazol-4-yl)methyl)carbamoyl)phenyl)sulfonyl)-1,6-dihydropyridine-3-carboxamide.

In one embodiment, the present invention provides a compound of formula (I) as described herein, or a pharmaceutically acceptable salt thereof, wherein the compound of formula (I) is selected from:
N-(9-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-9-oxononyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(5-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-5-oxopentyl)-1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamide;
1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(6-((7-nitrobenzo[c][1,2,5]oxadiazol-4-yl)amino)hexanoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamide;
1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate;
3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)thio)octanamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)sulfinyl)octanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide; and
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide.

In a preferred embodiment, the present invention provides a compound of formula (I) as described herein, or a pharmaceutically acceptable salt thereof, wherein the compound of formula (I) is selected from:
1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide;
1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate;
3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide; and
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide.

In a particularly preferred embodiment, the present invention provides a compound of formula (I) as described herein, or a pharmaceutically acceptable salt thereof, wherein the compound of formula (I) is 1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate.

In a particularly preferred embodiment, the present invention provides a compound of formula (I) as described herein, or a pharmaceutically acceptable salt thereof, wherein the compound of formula (I) is N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide.

In a particularly preferred embodiment, the present invention provides a compound of formula (I) as described herein, or a pharmaceutically acceptable salt thereof, wherein the compound of formula (I) is N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide.

In a particularly preferred embodiment, the present invention provides a compound of formula (I) as described herein, or a pharmaceutically acceptable salt thereof, wherein the compound of formula (I) is N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide.

In a particularly preferred embodiment, the present invention provides a compound of formula (I) as described herein, or a pharmaceutically acceptable salt thereof, wherein the compound of formula (I) is N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide.

In a particularly preferred embodiment, the present invention provides a compound of formula (I) as described herein, or a pharmaceutically acceptable salt thereof, wherein the compound of formula (I) is 1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate.

In a particularly preferred embodiment, the present invention provides a compound of formula (I) as described herein, or a pharmaceutically acceptable salt thereof, wherein the compound of formula (I) is 3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide.

In a particularly preferred embodiment, the present invention provides a compound of formula (I) as described herein, or a pharmaceutically acceptable salt thereof, wherein the compound of formula (I) is N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide.

In a particularly preferred embodiment, the present invention provides a compound of formula (I) as described herein, or a pharmaceutically acceptable salt thereof, wherein the compound of formula (I) is N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide.

In a particularly preferred embodiment, the present invention provides a compound of formula (I) as described herein, or a pharmaceutically acceptable salt thereof, wherein the compound of formula (I) is N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide.

In a particular embodiment, the present invention provides pharmaceutically acceptable salts of the compounds according to formula (I) as described herein. In a further particular embodiment, the present invention provides compounds according to formula (I) as described herein as free bases.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, ACN=acetonitrile, Boc=tert-butyloxycarbonyl, CAS RN=chemical abstracts registration number, CH₂Cl₂=dichloromethane, COMU=(1-Cyano-2-ethoxy-2-oxoethylidenaminooxy)dimethylamino-morpholino-carbenium hexafluorophosphate, [Cu(MeCN)₄]PF₆=Tetrakis(acetonitrile)copper(I) hexafluorophosphate, DCC=N,N′-Dicyclohexylcarbodiimide, DIC=N,N′-Diisopropylcarbodiimide, N,N′-Dicyclohexylcarbodiimide, DMAP=4-dimethylaminopyridine, DMA=N,N-dimethylacetamide, DMF=N,N-dimethylformamide, DMSO=dimethylsulfoxide, i-Pr₂NEt=N,N-diisopropylethylamine, EDCI=N-(3-dimethylaminopropyl)-N′-ethylcarbodiimide, EDCI·HCl=N-(3-dimethylaminopropyl)-N′-ethylcarbodiimide hydrochloride, ESI=electrospray ionization, Et₃N=triethylamine, Et₂O=diethyl ether, EtOAc=ethyl acetate, EtOH=ethanol, h=hour(s), GABA=γ-Aminobutyric acid, H₂O=water, HATU=1-[bis(dimethylamino)methylene]-1H-1,2,3-triazolo[4,5-b]pyridinium-3-oxide hexafluorophosphate, HCl=hydrochloric acid HOAt=1-Hydroxy-7-azabenzotriazole, HOBt=1-hydroxy-1H-benzotriazole; HPLC=high performance liquid chromatography, KO^{t}Bu=Potassium tert-butoxide, K₂CO₃=potassium carbonate, MeOH=methanol, MgSO₄=magnesium sulfate, min=minute(s), mL=milliliter, MS=mass spectrum, NaH=sodium hydride, NaHCO₃=sodium hydrogen carbonate, NaOH=sodium hydroxide, Na₂SO₄=sodium sulfate, NEt₃=triethylamine (TEA), NH₄Cl=ammonium chloride, OtBu=tert-butoxy, PG=protective group, PyAOP=(7-Azabenzotriazol-1-yloxy)tripyrrolidinophosphonium hexafluorophosphate, PyBOP=(Benzotriazol-1-yloxy)tripyrrolidinophosphonium hexafluorophosphate, R=any group, rt=room temperature, SiO₂=silicon dioxide, T3P=Propylphosphonic anhydride, TBTU=0-(Benzotriazol-1-yl)-N,N,N′,N′-tetramethyluronium tetrafluoroborate, TFA=trifluroacetic acid, THF=tetrahydrofuran, TLC=thin layer chromatography.

Compound of formula I wherein R⁴, R⁷, p, r, s, t, W, are as described herein can be synthesized in analogy to literature and/or as depicted for example in Scheme 1.

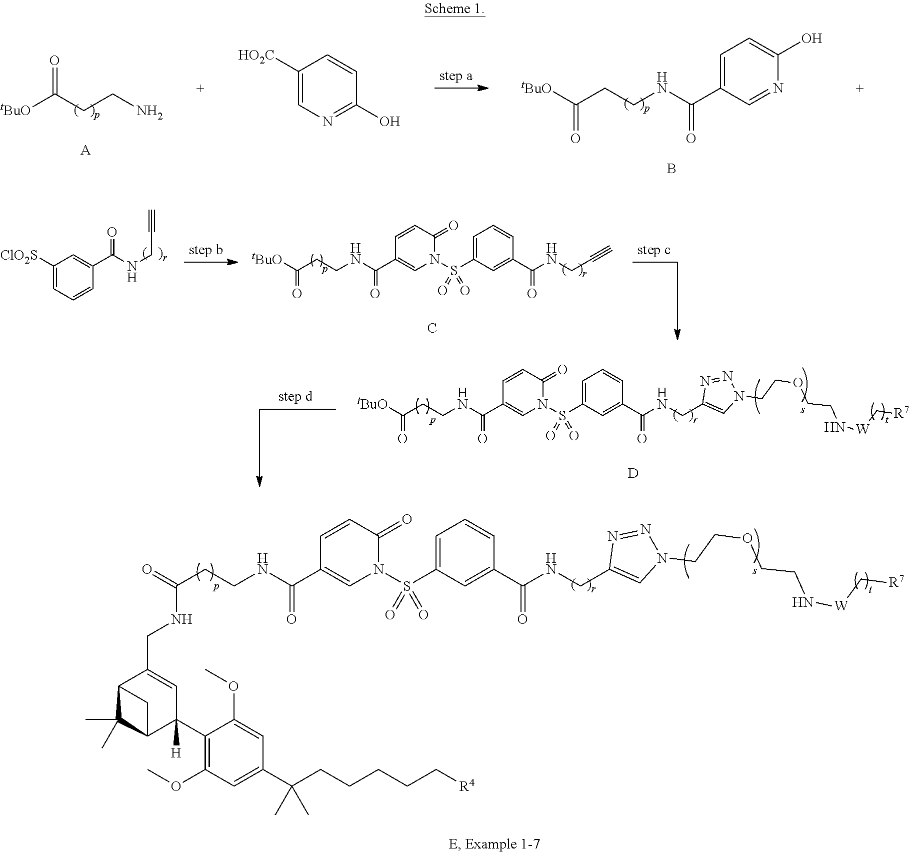

Accordingly, ω-amino ester linkers type A are reacted with 6-hydroxynicotinic acid to give B (step a). Amide couplings of this type can be accomplished by using one of the well-known coupling reagents such as, DCC, HATU, EDCI, TBTU, T3P, etc. and a base like i-Pr₂NEt, Et₃N or DMAP in a suitable solvent like DMF, DMA, CH₂Cl₂ or dioxane, preferably between 0° C. and room temperature. Subsequently, B is reacted with an activated sulfonate, such as, but not limited to sulfonyl halide in the presence of a base, preferably, KO^{t}Bu but also i-Pr₂NEt, TEA, DMAP in a suitable solvent, such as, THF or CH₂Cl₂ or dioxane to give C (step b). A person skilled in the art will acknowledge that the order of the addition of the reagents and the selection of base and solvent can be important in this type of reactions due to the reactivity and stability of the two tautomers of deprotonated 2-pyridone anions resulting in the formation of N- or O-sulfonylpyridone constitutional isomers. Compounds type C can be directly linked to a ligand to be used in a covalent transfer of a universal cargo such as alkyne to be further functionalized by secondary conjugation such as with an azide in the presence of a metal catalyst. Alternatively, the N-pyridone motif type C can be coupled by a [3+2]-azide-alkyne cycloaddition, optionally catalyzed by a metal catalyst such as Cu^{I} to give functionalized N-pyridone scaffold type D (step c). The use of a protic non-nucleophilic solvent mixture is preferential (CH₂Cl₂, AcOH) allowing the reaction to proceed at ambient temperature at higher yields, however not essential as for example MeOH, CH₂Cl₂ at 40° C. also works. The functionality is preferentially a fluorescent label but can also be any cargo for secondary reaction in vitro or in vivo, preferentially bioorthogonal, such as, strained alkene, strained alkyne, tetrazene, alkyne, azide, diazirine, aldehyde, boronic acid. Compounds type D are subsequently deprotected by acid such as HCl or TFA in a range of solvents such as CH₂Cl₂, Et₂O, dioxane and the acid coupled without further required purification in one step to a desired Protein-of-Interest ligand to yield E (step d). The ligand carries an amino functional group to be coupled by one of the well known coupling reagents (DCC, EDCI, TBTU, T3P), preferentially HATU, PyAOP or generally an activating reagent that yields an HOAt ester from the carboxylic acid. The reaction is run in a suitable solvent like DMF, DMA, CH₂Cl₂ or dioxane and an acid/base buffer system, such as, i-Pr₂NEt, TFA preferably between 0° C. and room temperature.

A key part of this invention is the discovery that the N-sulfonyl pyridone C can be formed virtually exclusively over the O-pyridone constitutional isomer using KO^{t}Bu in THF. Another key part of this invention is the discovery and use of the acid/base buffer system such as TFA/i-Pr₂NEt in the amide bond formation step with an N-sulfonyl pyridone present.

Preferentially, HATU mediated reaction is applied with a TFA/i-Pr₂NEt buffer system (step d). The HOAt active ester facilitates rapid formation of the amide bond, however the free HOAt if left unbuffered reacts with the electrophilic N-sulfonyl pyridone and mediates degradation. Selecting an appropriate acid/base buffer system prevents this critical side reaction and allows the final amide bond formation to proceed in the presence of the highly electrophilic motif. A person skilled in the art will acknowledge that similar reactivity would be anticipated for other coupling reagents where the activating agent following the bond formation is sufficiently acidic and nucleophilic to cleave the electrophile, such as TBTU, PyAOP, PyBOP, COMU.

A person skilled in the art will acknowledge that the order of the functionalization of C is interchangeable and depends upon desired application as well as availability of either functionalizing component.

Compound of formula I wherein R⁴, R⁵, p, q are as described herein can be synthesized in analogy to literature and/or as depicted for example in Scheme 2.

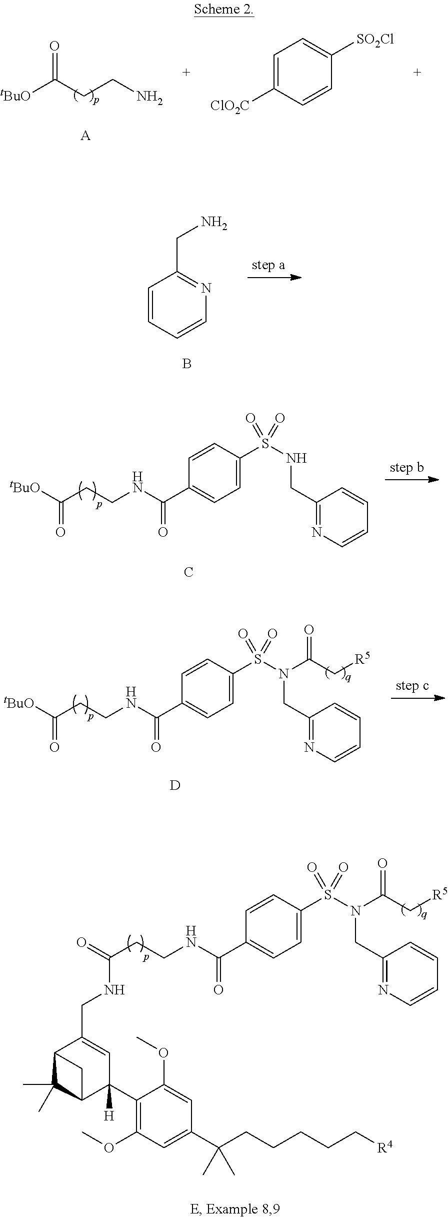

Accordingly, ω-amino ester linkers type A are reacted with activate 4-sulfobenzoic acid, such as in the form of acyl, sulfonyl halide, followed by addition of amine B to give compounds of formula C (step a). Subsequently, N-alkyl sulfonamide C is reacted with a functionalized acid to yield the N-acyl-N-alkyl sulfonamide D (step b). Amide couplings of this type can be accomplished by using one of the well-known coupling reagents such as, DCC, HATU, EDCI, TBTU, T3P, etc. and a base like i-Pr₂NEt, Et₃N or DMAP in a suitable solvent like DMF, DMA, CH₂Cl₂ or dioxane, preferably between 0° C. and room temperature. The compounds type D are subsequently deprotected by acid such as HCl or TFA in a range of solvents such as CH₂Cl₂, Et₂O, dioxane and the acid coupled without further required purification in one step to a desired Protein-of-Interest ligand to yield E (step c). The ligand carries an amino functional group to be coupled by one of the well known coupling reagents (DCC, EDCI, TBTU, T3P), preferentially HATU, PyAOP or generally an activating reagent that yields an HOAt ester from the carboxylic acid. The reaction is run in a suitable solvent like DMF, DMA, CH₂Cl₂ or dioxane and an acid/base buffer system, such as, i-Pr₂NEt, TFA preferably between 0° C. and room temperature.

Compound of formula I wherein R⁴, R⁵, p, q are as described herein can be synthesized in analogy to literature and/or as depicted for example in Scheme 3.

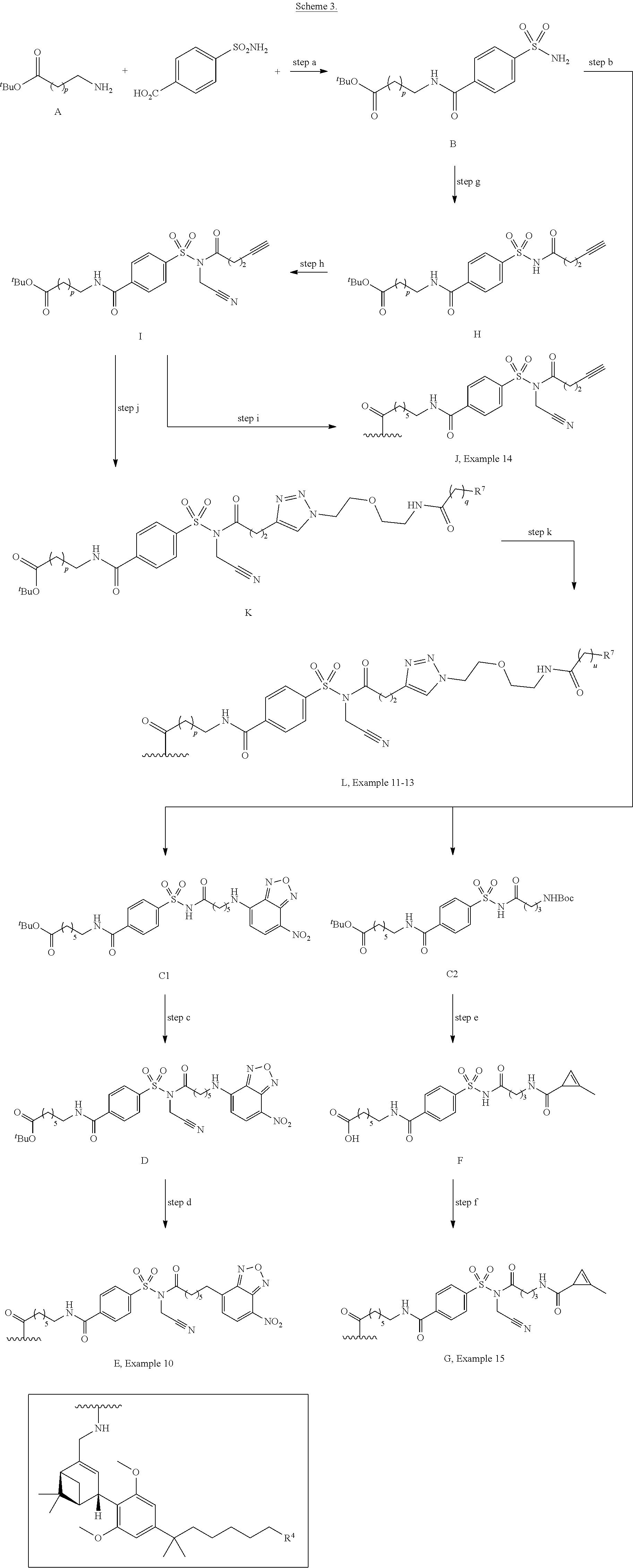

Accordingly, ω-amino ester linkers type A are reacted with 4-sulfamoylbenzoic acid to yield B (step a). Amide couplings of this type can be accomplished by using one of the well-known coupling reagents such as, DCC, HATU, EDCI, TBTU, T3P, etc. and a base like i-Pr₂NEt, Et₃N or DMAP in a suitable solvent like DMF, DMA, CH₂Cl₂ or dioxane, preferably between 0° C. and room temperature, preferably catalyzed by HOBt. Subsequently, the sulfonamide of B is N-acylated with a carboxylic acid and one of the well-known coupling reagents such as, DCC, HATU, EDCI, TBTU, T3P, etc. and a base like i-Pr₂NEt, Et₃N, preferably catalyzed by DMAP in a suitable solvent like DMF, DMA, CH₂Cl₂ or dioxane, preferably between 0° C. and room temperature to yield C₁ and C₂ (step b).

Following pathway towards Example 10, the N-acyl sulfonamide C₁ is alkylated with iodoacetonitrile, assisted with a suitable base, to yield compounds type D (step c). The tert-butyl esters of compounds type D are subsequently deprotected by acid such as HCl or TFA in a range of solvents such as CH₂Cl₂, Et₂O, dioxane and the acid coupled without further required purification in one step to a desired Protein-of-Interest ligand to yield E (step d). The ligand carries an amino functional group to be coupled by one of the well known coupling reagents (DCC, EDCI, TBTU, T3P), preferentially HATU, PyAOP or generally an activating reagent that yields an HOAt ester from the carboxylic acid. The reaction is run in a suitable solvent like DMF, DMA, CH₂Cl₂ or dioxane and an acid/base buffer system, such as, i-Pr₂NEt, TFA preferably between 0° C. and room temperature.

Following pathway towards Example 15, C₂ is deprotected with an acid, such as, HCl or TFA and reacted with an activated carboxylic acid derivative, such as, N-hydroxysuccinimide ester or an acid anhydride, to yield N-acyl sulfonamides F (step e). The compounds type F are subsequently coupled to a desired Protein-of-Interest ligand. The ligand carries a complementary functional group such as an amine or an alcohol to be coupled by one of the well known coupling reagents DCC, HATU, EDCI, TBTU, T3P, etc. and a base like i-Pr₂NEt, Et₃N or DMAP in a suitable solvent like DMF, DMA, CH₂Cl₂ or dioxane, preferably between 0° C. and room temperature. Finally, alkylation of the intermediate N-acyl sulfonamide with a desired alkyl halide furnishes the probe G (step f).

Synthesis towards Example 11-14, in step g, B is acylated with 4-pentynoic acid to give H under conditions analogous to step b. In step h, the N-acyl sulfonamide in H is alkylated under analogous conditions to step c, to yield activated N-acyl-N-alkyl sulfonamide platform reagent I.

Following pathway towards Example 14, I is deprotected by acid such as HCl or TFA in a range of solvents such as CH₂Cl₂, Et₂O, dioxane and the acid coupled without further required purification in one step to a desired Protein-of-Interest ligand (step i).

In synthesis of Examples 11-13, the alkyne of I is functionalized with an azide bearing cargo, such as a fluorophore, in an analogous fashion to Scheme 1, step c, to yield K (step j). K is subsequently deprotected and coupled with a desired ligand, analogous conditions to Scheme 1, step d, to bear the final compounds type L (step k).

A key part of this invention is the discovery and use of an acid/base buffer system, such as TFA/i-Pr₂NEt, in the amide bond formation step with N-acyl-N-alkyl sulfonamide present. Preferentially, HATU mediated reaction is applied with a TFA/i-Pr₂NEt buffer system. The HOAt active ester facilitates rapid formation of the amide bond, however the free HOAt if left unbuffered reacts with the electrophilic N-acyl-N-alkyl sulfonamide and mediates degradation. Selecting an appropriate acid/base buffer system prevents this critical side reaction and allows the final amide bond formation to proceed in the presence of the highly electrophilic motif. A person skilled in the art will acknowledge that similar reactivity would be anticipated for other coupling reagents where the activating agent following the bond formation is sufficiently acidic and nucleophilic to cleave the electrophile, such as TBTU, PyAOP, PyBOP, COMU.

A person skilled in the art will acknowledge that the amide bond formation relies on the differences in acidity of the nucleophilic catalyst activating agent (e.g. HOAt), the carboxylic acid and the acid/base (e.g. TFA/i-Pr₂NEt) buffer system. The same reaction outcome would be expected should a different activating agent be applied with a matched buffer system. In the aforementioned case, acidity of HOAt (pKa≈3.3) lies between the carboxylic acid (pKa≈5) and TFA (pKa≈0.5). As such, during the reaction any released nucleophilic HOAt anion is immediately protonated by TFA and therefore does not participate in the nucleophilic degradation of the electrophilic motif. The resulting non-nucleophilic TFA anion does not cleave the electrophilic motif. With the above exemplified logic a person skilled in the art can construct a matching system to their preferred coupling reagent and buffer system.

A person skilled in the art will acknowledge that the order of the functionalization of I is interchangeable and depends upon desired application as well as availability of either functionalizing component.

Compound of formula I wherein R¹, R⁴, p, X are as described herein can be synthesized in analogy to literature and/or as depicted for example in Scheme 4.

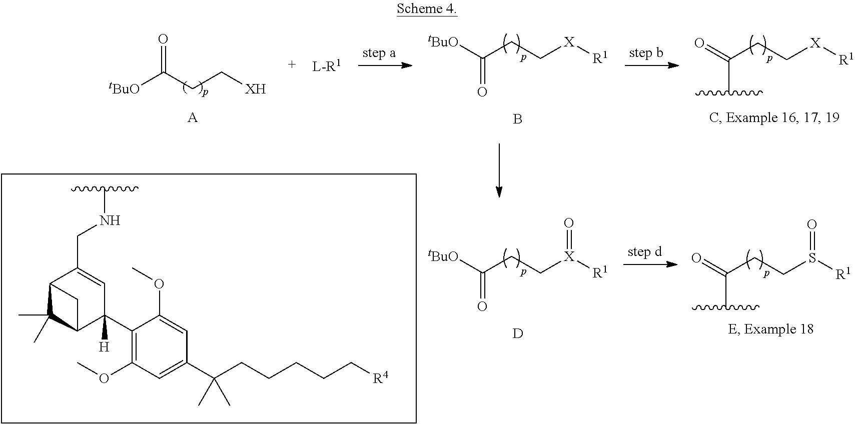

Accordingly, esters type A where X can be N, O, S, Se, P react with a fluorophore carrying a leaving group (L) to give compounds type B (step a). The ester of B is subsequently cleaved with an acid using standard conditions to reveal the carboxylic acid which is coupled to a desired targeting ligand to yield compounds type C with analogy to methods described previously herein (step b). Alternatively, B can be oxidized to yield compounds type D (step c). This can be achieved by oxidants such as hydrogen peroxide, meta-chloroperoxybenzoic acid, dimethyldioxirane or oxone. In an analogous fashion to C the compounds type E are obtained following deprotection and amide bond formation with the ligand of interest (step d).

In one aspect, the present invention provides a process for manufacturing a fluorescent probe P1 or P2 for a protein of interest,

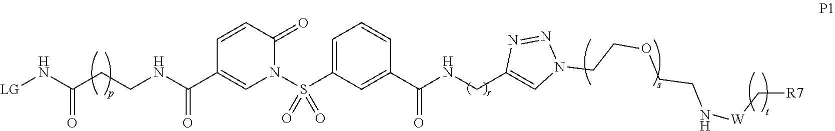

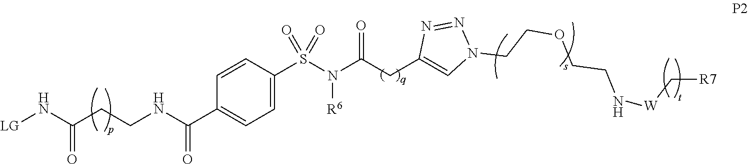

wherein LG is a ligand for a protein of interest, and W, R⁶, R⁷, p, q, r, and s are as defined herein, comprising:
   (a) reacting an alkyne C or I

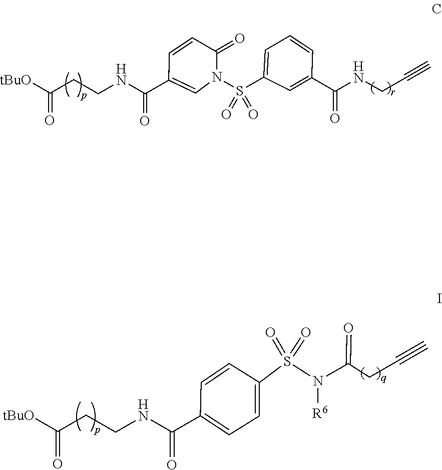

wherein R⁶, p, q, and r are as defined herein, with an azide-substituted fluorophore M

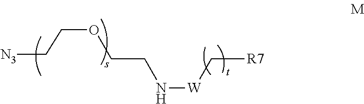

wherein W, R⁷, s, and t are as defined herein;
to form a compound D or K

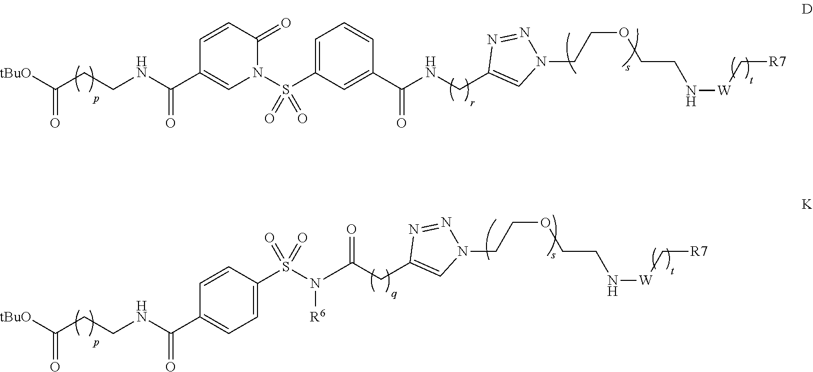

wherein W, R⁶, R⁷, p, q, r, and s are as defined herein;
followed by
(b) removing the t-Bu protective group from compound D or K using an acid, for example HCl or TFA, to afford compound D1 or K1

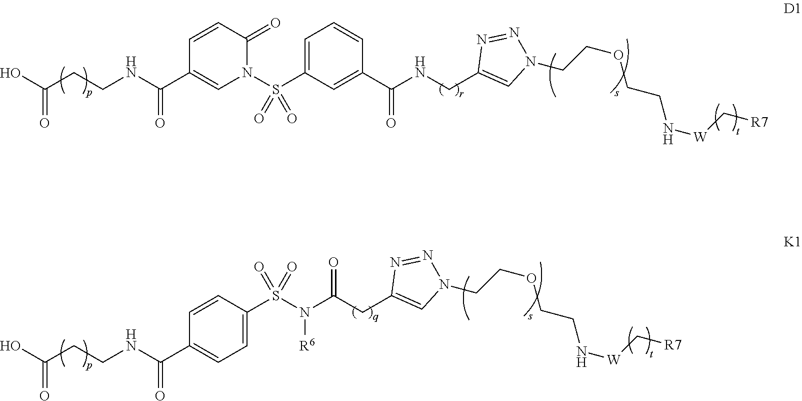

wherein W, R⁶, R⁷, p, q, r, and s are as defined herein;
followed by
(c) reacting compound D1 or K1 with an amino-substituted ligand for a protein of interest N,

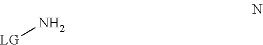

wherein LG is a ligand for a protein of interest,
in the presence of a coupling reagent and an acid/base buffer system;
to afford said fluorescent probe P1 or P2 for a protein of interest.

In the process according to the invention described above, the reaction sequence may be reversed, such that steps (b) and (c) are performed prior to step (a).

Thus, in a further aspect, the present invention provides a process for manufacturing a fluorescent probe P1 or P2 for a protein of interest,

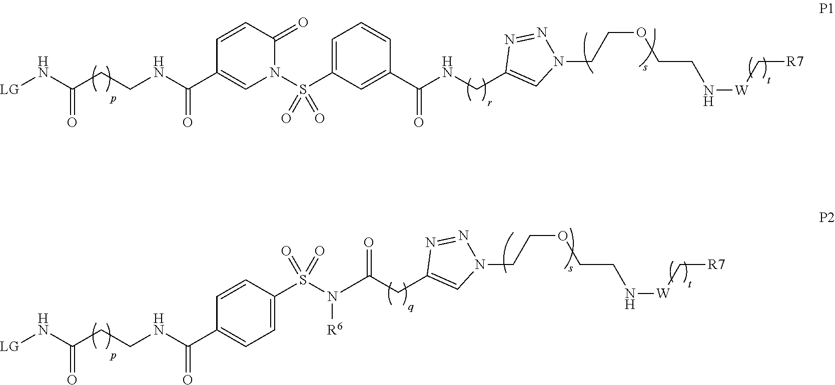

wherein LG is a ligand for a protein of interest, and W, R⁶, R⁷, p, q, r, and s are as defined herein, comprising:
   (a) removing the t-Bu protective group from a compound C or I using an acid, for example HCl or TFA,

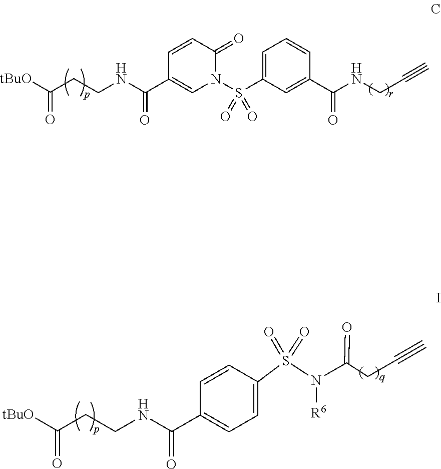

wherein R⁶, p, q, and r are as defined herein,
to afford a compound Ci or Ii

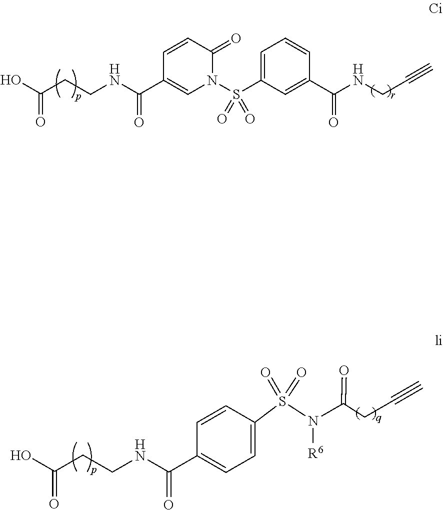

wherein R⁶, p, q, and r are as defined herein,
followed by
(b) reacting said compound Ci or Ii with an amino-substituted ligand for a protein of interest N,

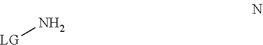

wherein LG is a ligand for a protein of interest,
in the presence of a coupling reagent and an acid/base buffer system;
to afford a compound Cii or Iii

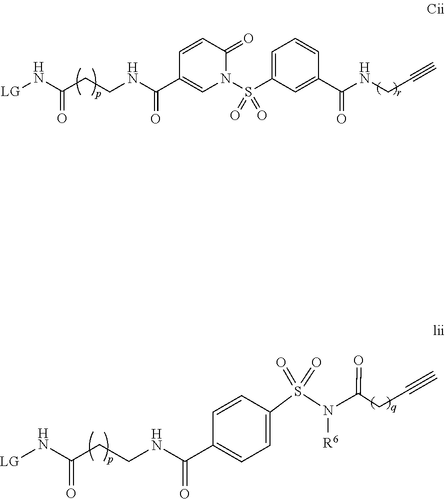

wherein LG is a ligand for a protein of interest, and R⁶, p, q, and r are as defined herein;
followed by
(c) reacting said compound Cii or Iii with an azide-substituted fluorophore M

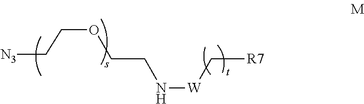

wherein W, R⁷, s, and t are as defined herein;
to afford said fluorescent probe P1 or P2 for a protein of interest.

In a further aspect, the present invention provides a process for manufacturing a fluorescent probe P1 or P2 for a protein of interest,

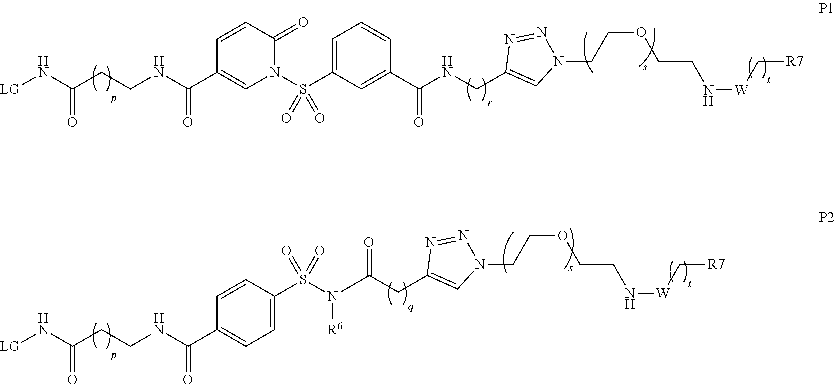

wherein LG is a ligand for a protein of interest, and W, R⁶, R⁷, p, q, r, and s are as defined herein, comprising:
   (a) reacting a compound D1 or K1

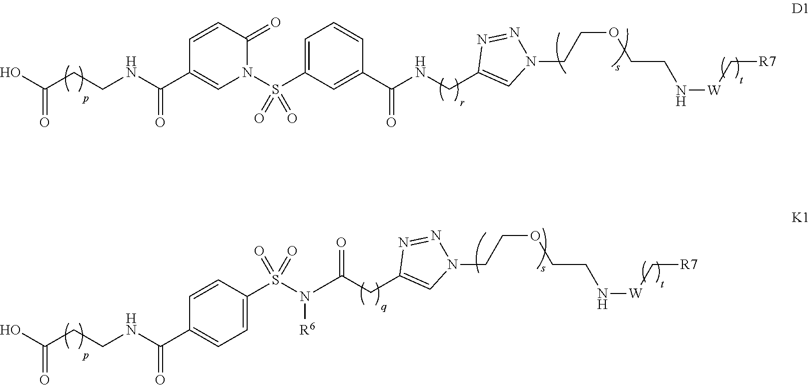

wherein W, R⁶, R⁷, p, q, r, and s are as defined herein;
with an amino-substituted ligand for a protein of interest N,

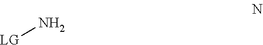

wherein LG is a ligand for a protein of interest,
in the presence of a coupling reagent and an acid/base buffer system;
to afford said fluorescent probe P1 or P2 for a protein of interest.

In a further aspect, the present invention provides a process for manufacturing a fluorescent probe P1 or P2 for a protein of interest,

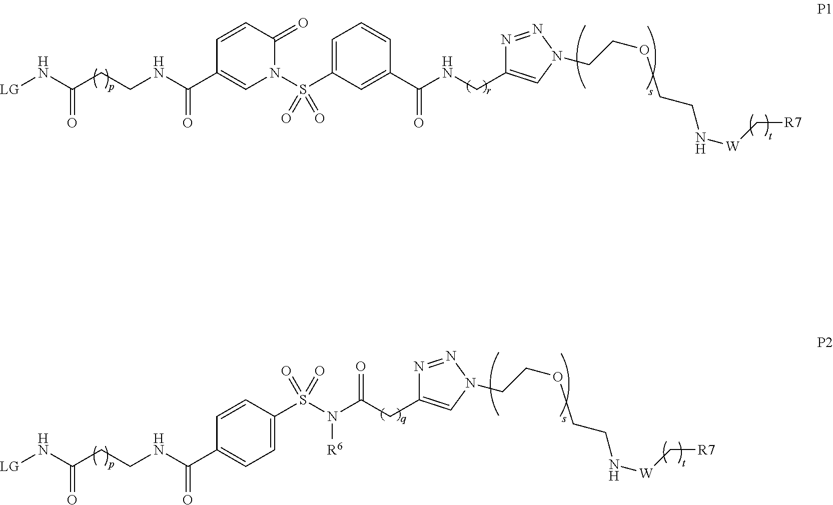

wherein LG is a ligand for a protein of interest, and W, R⁶ R⁷, p, q, r, and s are as defined herein, comprising:
   (a) reacting a compound Cii or Iii

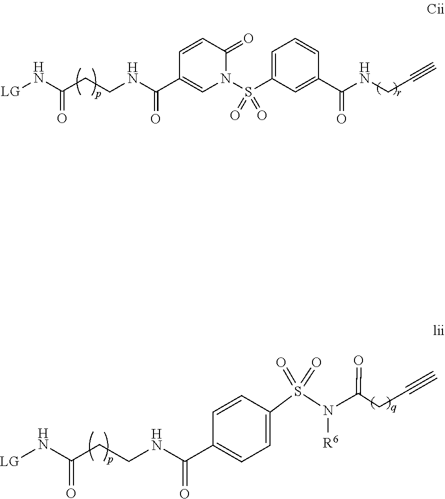

wherein LG is a ligand for a protein of interest, and R⁶, p, q, and r are as defined herein;
with an azide-substituted fluorophore M

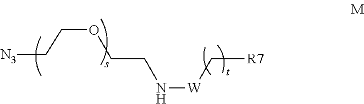

wherein W, R⁷, s, and t are as defined herein;
to afford said fluorescent probe P1 or P2 for a protein of interest.

In one aspect, the present invention provides a process for manufacturing a compound D or K,

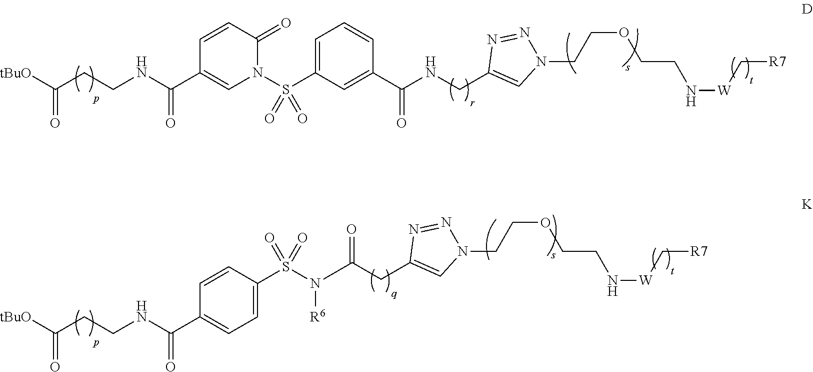

wherein W, R⁶, R⁷, p, q, r, and s are as defined herein, comprising:
   (a) reacting an alkyne C or I

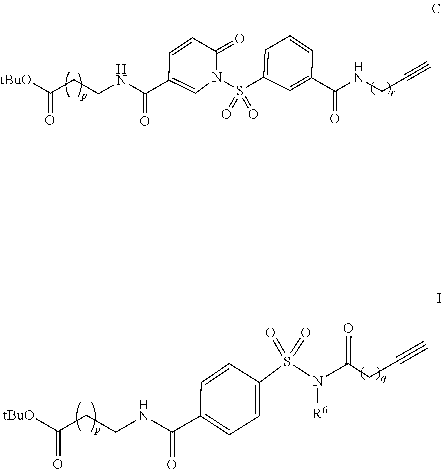

wherein R⁶, p, q, and r are as defined herein, with an azide-substituted fluorophore M

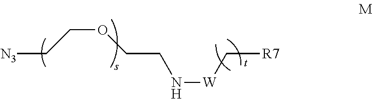

wherein W, R⁷, s, and t are as defined herein;
to form said compound D or K.

In a further aspect, the present invention provides a process for manufacturing a compound Cii or Iii,

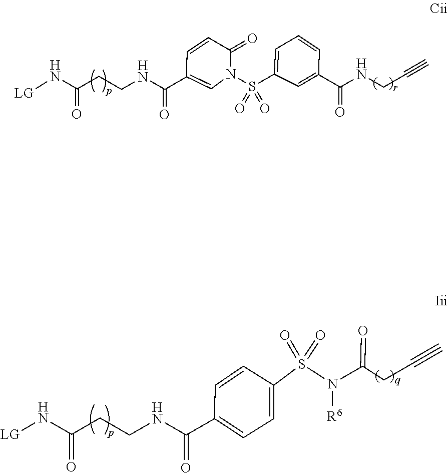

wherein LG is a ligand for a protein of interest, and R⁶, p, q, and r are as defined herein, comprising:
   (a) reacting said compound Ci or Ii

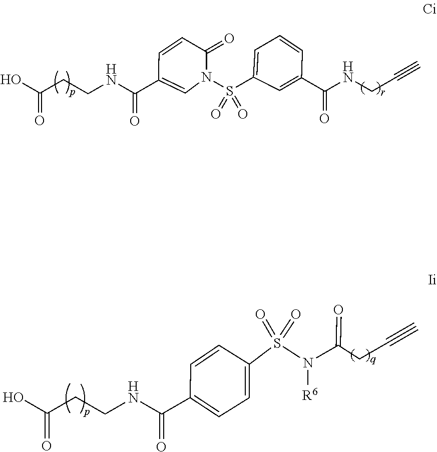

wherein R⁶, p, q, and r are as defined herein,
with an amino-substituted ligand for a protein of interest N,

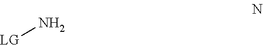

wherein LG is a ligand for a protein of interest,
in the presence of a coupling reagent and an acid/base buffer system;
to afford said compound Cii or Iii.

In a further aspect, the present invention provides a process for manufacturing amides of general formula 1

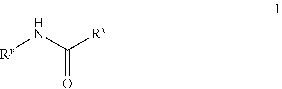

wherein R^{x} and R^{y} are both chemical moieties that are attached to the parent amide functional group through a carbon atom; and
wherein R^{x} comprises an N-sulfonyl pyridone (SP) motif i, or an N-acyl-N-alkyl sulfonamide (NASA) motif ii

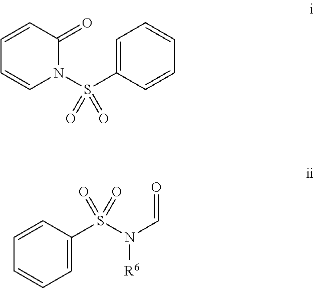

wherein R⁶ is as defined herein;
comprising:
reacting a carboxylic acid 2

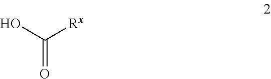

with an amine 3

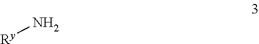

in the presence of a coupling reagent and an acid/base buffer system;
to afford said amide 1.

In one embodiment, the coupling reagent used in the amide coupling according to the invention is selected from DCC, PyBOP, COMU, EDCI, TBTU, and T3P.

In a preferred embodiment, the coupling reagent used in the amide coupling according to the invention yields an HOAt ester from the carboxylic acid in the presence of a suitable catalyst. Examples of coupling reagents that yield an HOAt ester from the carboxylic acid in the presence of a suitable catalyst are well known in the art. Non-limiting examples include DCC and HOAt. Likewise, suitable catalysts that are used in this type of coupling reactions are well known in the art. Non-limiting examples include EDCI and HOAt, and DIC and HOAt.

In a particularly preferred embodiment, the coupling reagent used in the amide coupling according to the invention is selected from HATU and PyAOP.

In a further particularly preferred embodiment, the coupling reagent used in the amide coupling according to the invention is HATU.

In one embodiment, 1-5 equivalents, for example 1, 2, 3, 4 or 5 equivalents of coupling reagent relative to the respective carboxylic acid are used in the amide coupling according to the invention.

In a preferred embodiment, 1-2 equivalents of coupling reagent relative to the respective carboxylic acid are used in the amide coupling according to the invention.

In a particularly preferred embodiment, 1.1 equivalents of coupling reagent relative to the respective carboxylic acid are used in the amide coupling according to the invention.

In a further particularly preferred embodiment, the coupling reagent used in the amide coupling according to the invention is PyAOP.

In one embodiment, 1 to 2 equivalents of coupling reagent relative to the respective carboxylic acid are used in the amide coupling according to the invention.

In a preferred embodiment, 1 to 1.5 equivalents of coupling reagent relative to the respective carboxylic acid are used in the amide coupling according to the invention.

In a particularly preferred embodiment, 1.1 equivalents of coupling reagent relative to the respective carboxylic acid are used in the amide coupling according to the invention.

In one embodiment, the acid/base buffer system used in the amide coupling according to the invention is TFA/i-Pr₂NEt.

In one embodiment, the acid/base buffer system used in the amide coupling according to the invention is TFA/i-Pr₂NEt, wherein the ratio of TFA to i-Pr₂NEt is 1/3-11/13, for example 1/3, 3/5, 5/7, 7/9, 9/11 or 11/13.

In a preferred embodiment, the acid/base buffer system used in the amide coupling according to the invention is TFA/i-Pr₂NEt, wherein the ratio of TFA to i-Pr₂NEt is 3/5-3.5/5.5.

In a particularly preferred embodiment, the acid/base buffer system used in the amide coupling according to the invention is TFA/i-Pr₂NEt, wherein the ratio of TFA to i-Pr₂NEt is 3.3/5.3.

In a preferred embodiment, the acid/base buffer system used in the amide coupling according to the invention is TFA/i-Pr₂NEt, wherein 3 to 3.5 equivalents of TFA and 5 to 5.5 equivalents of i-Pr₂NEt are used relative to the respective carboxylic acid.

In a particularly preferred embodiment, the acid/base buffer system used in the amide coupling according to the invention is TFA/i-Pr₂NEt, wherein 3.3 equivalents of TFA and 5.3 equivalents of i-Pr₂NEt are used relative to the respective carboxylic acid. In one embodiment, the acid/base buffer system used in the amide coupling according to the invention is TFA/i-Pr₂NEt, wherein 1-10 equivalents of TFA are used relative to the coupling reagent.

In a preferred embodiment, the coupling reagent used in the amide coupling according to the invention is HATU and the acid/base buffer system is TFA/i-Pr₂NEt.

In one embodiment, the amide coupling according to the invention is run in a solvent selected from DMF, DMSO, DMA, CH₂Cl₂ and dioxane, or a mixture thereof.

In a preferred embodiment, the amide coupling according to the invention is run in a mixture of DMF and CH₂C₁₂.

In a preferred embodiment, the ligand for a protein of interest is a ligand for CB2, in particular the following ligand:

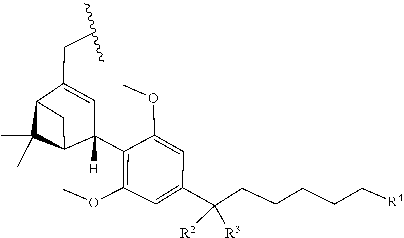

wherein R², R³, and R⁴ are as defined herein, and the wavy line indicates the point of attachment of the ligand to the remainder of the compound of formula (I) as described herein.

In a further aspect, the present invention provides a compound selected from

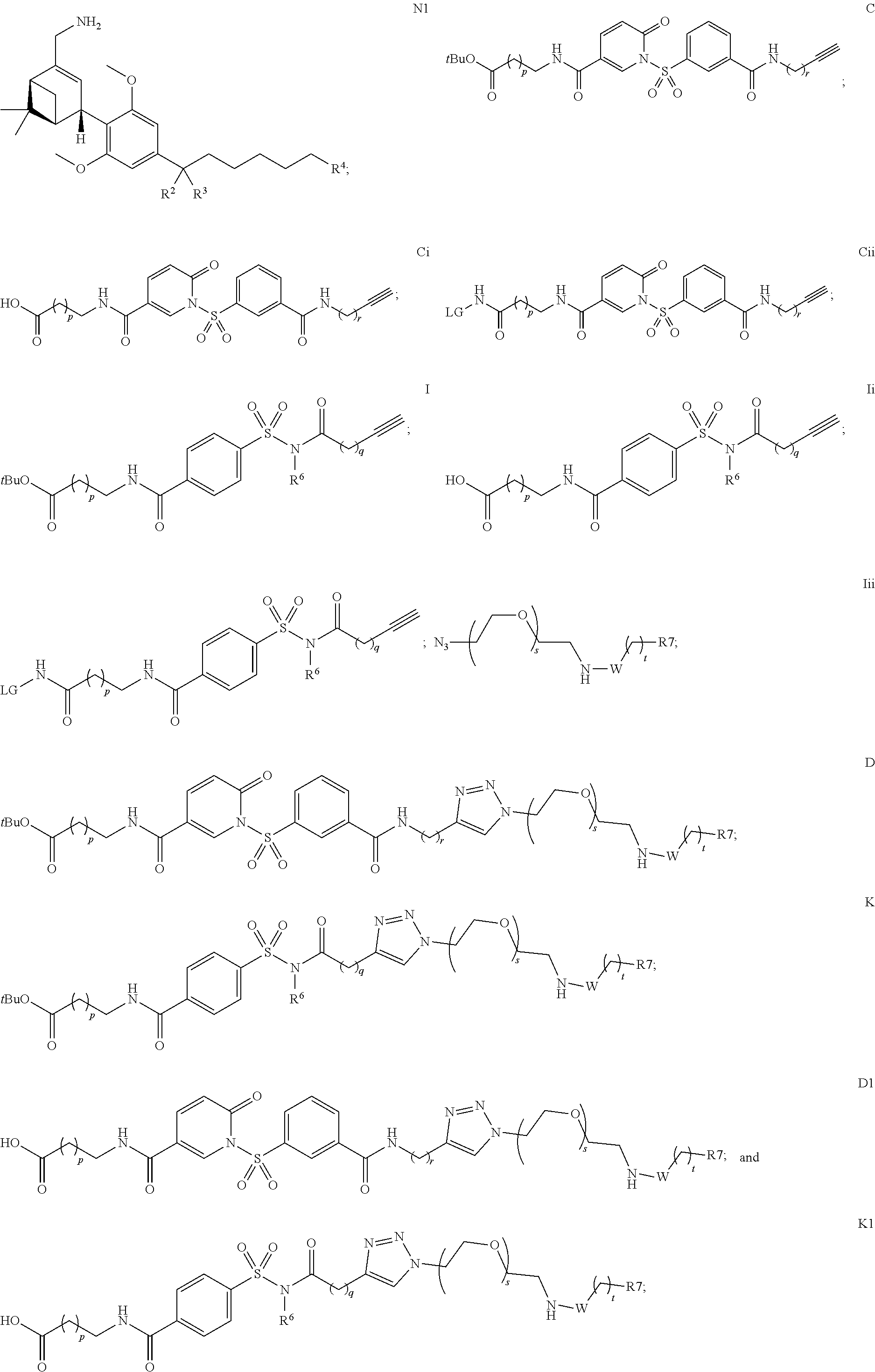

wherein R², R³, R⁴, R⁶, R⁷, p, q, r, s, t are as described herein and LG is a ligand for a protein of interest.
TR-FRET hCB2R Binding Assay

### Cell Culture:

Cells were maintained in a humidified environment at 37° C. and 5% CO₂ in Dulbecco's modified Eagle's medium (DMEM) with 10% fetal bovine serum (FBS) containing blasticidin (5 pg/mL; Invitrogen) and (Zeocin; 20 μg/mL; Invitrogen). For inducible expression, SNAP-tagged human CB₂ receptor cDNAs, in pcDNA4/TO were introduced through transfection, using PEI into HEK293TR cells (Invitrogen, which express Tet repressor protein to allow inducible expression). A mixed population stable line was selected by resistance to blasticidin (TR vector, 5 μg/mL) and Zeocin; (receptor plasmid, 20 μg/mL). For receptor-inducible expression, cells were seeded into t175 cm² flasks, grown to 70% confluence and DMEM containing 1 μg/ml tetracycline added. 24 h later cells were labelled with SNAP-Lumi4-Tb (CisBio) and membranes prepared as described in detail below.

### Terbium Labeling of SNAP-Tagged CB2R HEK293-TR Cells:

Cell culture medium was removed from the t175 cm2 flasks containing confluent adherent CB₂ HEK293-TR cells. Cells were washed 1× in PBS (GIBCO Carlsbad, CA) followed by 1× Tag-lite labeling medium (LABMED, CisBio) to remove the excess cell culture media, then ten millilitre of LABMED containing 100 nM of SNAP-Lumi4-Tb was added to the flask and incubated for 1 h at 37° C. under 5% CO₂. Cells were washed 1× in PBS (GIBCO Carlsbad, CA) to remove the excess of SNAP-Lumi4-Tb then detached using 5 mL of GIBCO enzyme-free Hank's-based cell dissociation buffer (GIBCO, Carlsbad, CA) and collected in a vial containing 5 ml of DMEM (Sigma-Aldrich) supplemented with 10% fetal calf serum. Cells were pelleted by centrifugation (5 min at 1500 rpm) and the pellets were frozen to −80° C. To prepare membranes, homogenization steps were conducted at 4° C. (to avoid receptor degradation) as described in C. K. Herenbrink, et. al., Nat. Commun., 2016, 7, 10842.

### Fluorescent Ligand-Binding Assays:

All fluorescent ligand binding experiments were conducted in white 384-well Optiplate plates, in assay binding buffer, either Hanks Balanced Salt Solution (HBSS), 5 mM HEPES, 0.5% BSA, 0.02% pluronic F-127 pH 7.4, and 100 μM GppNHp. GppNHp was included to remove the G protein-coupled population of receptors that can result in two distinct populations of binding sites in membrane preparations, since the Motulsky-Mahan model is only appropriate for ligands competing at a single site. In all cases, nonspecific binding was determined by the presence of 1 μM SR144528. Data are presented in mean±SEM from a representative of 3-8 experiments.

### Determination of Fluorescent Ligand Binding Kinetics and Equilibrium Affinity:

To accurately determine association rate (kon) and dissociation rate (koff) values, the observed rate of association (kob) was calculated using at least five different concentrations 8-SiR (for 8-SiR and assay description see T. Gazzi et al. Chem. Sci., 2022,13, 5539-5545). The appropriate concentration of fluorescent ligand binding was incubated with human CB2R HEK293-TR cell membranes (4 pg per well) in assay binding buffer (final assay volume, 40 μL). The degree of fluorescent ligand bound to the receptor was assessed at multiple time points by HTRF detection to allow construction of association kinetic curves. The resulting data were globally fitted to the association kinetic model (Eq. 1, see signal detection and data analysis section below) to derive a single best-fit estimate for kon and koff as described under data analysis. Saturation analysis was performed at equilibrium, by simultaneously fitting total and Nonspecific (NSB) binding data (Eq. 2, see signal detection and data analysis section below) allowed the determination of fluorescent ligand binding affinity.

### Competition Binding:

To determine the affinity of CB₂R-specific ligands, we used a simple competition kinetic binding assay. This approach involves the simultaneous addition of both fluorescent ligand and competitor to the CB₂R preparation. 62.5 nM 8-SiR, concentration which avoid ligand depletion in this assay volume, were added simultaneously with increasing concentrations of the unlabeled compound to CB₂R cell membranes (4 μg per well) in 40 μL of assay buffer in a 384-well plate incubated at room temperature with orbital mixing. The degree of fluorescent ligand bound to the receptor was assessed at equilibrium by HTRF detection. Nonspecific binding was determined as the amount of HTRF signal detected in the presence of SR144528 (1 μM) and was subtracted from total binding, to calculate specific binding for construction of IC₅₀ curves.

### Signal Detection and Data Analysis:

Signal detection was performed on a Pherastar FSX (BMG Labtech, Offenburg, Germany). The terbium donor was always excited with eight laser flashes at a wavelength of 337 nm. TR-FRET signals were collected at 665 (acceptor) and 620 nm (donor) when using the red acceptor fluorescent ligand 8-SiR. HTRF ratios were obtained by dividing the acceptor signal by the donor signal and multiplying this value by 10,000. All experiments were analyzed by non-regression using Prism 8.0 (GraphPad Software, San Diego, USA).

Fluorescent ligand association data were fitted as follows to a global fitting model using GraphPad Prism 8.0 to simultaneously calculate kₒₙ and k_{off} using the following equation,

$$Y = {Y}_{max} * ( 1 - exp ( - 1 * {k}_{ob} * X ) ) wherein , {k}_{o b} = [ L ] * {k}_{o n} + {k}_{off} \qquad ( Eq . l )$$

Where, k_{ob} equals the observed rate of ligand association and kₒₙ and k_{off} are the association and dissociation-rate constants respectively of the fluorescent ligand. In this globally fitted model of tracer binding, tracer concentrations [L] are fixed, kₒₙ and k_{off} are share parameters whilst k_{obs} is allowed to vary. Here, Y is the level of receptor-bound tracer, Yₘₐₓ is the level of tracer binding at equilibrium, X is in units of time (eg. min) and k_{obs} is the rate in which equilibrium is approached (eg. min⁻¹).

$$Total binding = Specific + NSB = \frac{B max * [ L ]}{Kd + [ L ]} + slope * [ L ] + Background \\ NSB = slope * [ L ] + Background \qquad ( Eq . 2 )$$

Saturation binding data were analyzed by non-linear regression according to one-site equation by globally fitting total and NSB. Individual estimates for the fluorescent ligand dissociation constant (Ka) were calculated using the following equations where L is the fluorescent ligand concentration:

Fitting the total and NSB data sets globally (simultaneously), sharing the value of slope, provides one best-fit value for both the Ka and the Bₘₐₓ.

Competition displacement binding data were fitted to sigmoidal (variable slope) curves using a ‘four-parameter logistic equation’:

$$Y = Bottom + ( Top - Bottom ) / ( 1 + 1 {0}^{( logIC 50 - X ) \cdot Hill coefficient} ) \qquad ( Eq . 3 )$$

IC₅₀ values obtained from the inhibition curves were converted to Kᵢ values using the method of Cheng and Prusoff (C. Yung-Chi, W. H. Prusoff, Biochem. Pharmacol. 1973, 22, 3099-3108).

TABLE 1

| Example | K_{d} hCB₂R [nM] |
|---|---|
| 1 | 9.1 |
| 2 | 22.9 |
| 3 | 24.2 |
| 4 | 4.5 |
| 5 | 59 |
| 6 | — |
| 7 | 8.9 |
| 8 | 12 |
| 9 | 35.5 |
| 10 | 3.9 |
| 11 | 152 |
| 12 | 25 |
| 13 | 124 |
| 14 | 1.5 |
| 15 | — |
| 16 | — |
| 17 | — |
| 18 | — |
| 19 | 2.0 |

### Using the Compounds of the Invention

The compounds of formula (I) are fluorescent or bioorthogonal probes with high affinity for CB₂R. They may thus be used as high-resolution tools to investigate localization, expression levels and protein distribution in health and disease, structure, dynamics and function of CB₂R in living cells. They may also be applied in flow cytometry fluorescence-activated cell sorting (FACS) experiments or cellular trafficking studies using confocal live cell imaging. The probes provide superb tools to conduct these experiments without disturbance to the cellular homeostasis due to their functionalization with a cleavable motif that allows dissociation of the ligand following covalent labeling.

Due to their covalent nature of labelling, the probes are especially well suited for imaging of low abundance target proteins. The probes can be used to construct FRET sensors of membrane-bound and intracellular protein targets and subsequently used to investigate ligand-protein interaction in real time by the means of TR-FRET.

In one aspect, the present invention provides a compound of formula (I) described herein, for use as fluorescent or a bioorthogonal probe for the cannabinoid receptor 2 (CB₂R).

In a further aspect, the present invention provides the use of a compound of formula (I) described herein as a fluorescent or a bioorthogonal probe for the cannabinoid receptor 2 (CB₂R).

In a further aspect, the present invention provides a method of imaging cannabinoid receptor 2 (CB₂R), comprising contacting said cannabinoid receptor 2 (CB₂R) with a compound of formula (I) described herein.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### N-(5-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-5-oxopentyl)-1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamide

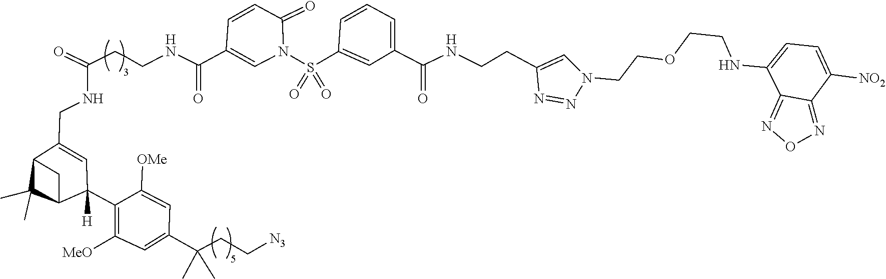

### Step a) tert-butyl 5-(6-hydroxynicotinamido)pentanoate

Solution of 6-hydroxynicotinic acid (155 mg, 1.11 mmol, 1.0 equiv, CAS RN 5006-66-6), tert-butyl 5-aminopentanoate (193 mg, 1.11 mmol, 1.0 equiv, CAS RN 63984-03-2), HOBt·H₂O (213 mg, 1.39 mmol, 1.25 equiv), EDCI·HCl (267 mg, 1.39 mmol, 1.25 equiv) and Et₃N (388 μL, 2.79 mmol, 2.5 equiv) in DMF (2.60 mL) was stirred for 16 h at ambient temperature. The mixture was concentrated in vacuoo and the crude product was purified by flash column chromatography (SiO₂, dry loading on SiO₂, 5% MeOH in CH₂Cl₂) to afford the title compound as a white solid (285 mg, 87%).

¹H NMR (500 MHz, CD₂Cl₂) δ 12.58 (bs, 1H), 8.10 (d, J=2.5 Hz, 1H), 7.86 (dd, J=9.5, 2.6 Hz, 1H), 7.54 (t, J=5.6 Hz, 1H), 6.43 (dd, J=9.5, 0.6 Hz, 1H), 3.49-3.22 (m, 2H), 2.22 (t, J=7.0 Hz, 2H), 1.69-1.52 (m, 4H), 1.40 (s, 9H). ¹³C NMR (126 MHz, CD₂Cl₂) δ 173.6, 165.0, 164.9, 140.4, 137.5, 119.6, 115.4, 80.7, 40.1, 35.5, 29.4, 28.4, 22.9. IR (neat, vₘₐₓ/cm⁻¹) 3311, 3058, 2978, 2930, 1724, 1654, 1632, 1611, 1542, 1460, 1428, 1392, 1367, 1313, 1251, 1148, 1096. HRMS (ESI): m/z=317.1475 [M+Na]⁺ (calc. for C₁₅H₂₂N₂NaO₄ m/z=317.1472)

### Step b) tert-butyl 5-(1-((3-(but-3-yn-1-ylcarbamoyl)phenyl)sulfonyl)-6-oxo-1,6-dihydropyridine-3-carboxamido)pentanoate

tert-butyl 5-(6-hydroxynicotinamido)pentanoate (15.0 mg, 51 μmol, 1.0 equiv) was dissolved in anhydrous THF (0.50 mL) at rt. and cooled to −78° C. KO^{t}Bu (1M in THF, 102 μL, 102 μmol, 2.0 equiv) was added dropwise at −78° C. and the solution was stirred for 10 min. at −78° C. Subsequently, 3-(but-3-yn-1-ylcarbamoyl)benzenesulfonyl chloride (41.5 mg, 153 μmol, 3.0 equiv, CAS RN 1344252-51-2) dissolved in anhydrous THF (0.30 mL) was added dropwise and the mixture was stirred at −78° C. for 2 h before it was allowed to warm up to rt. The solvent was removed in vacuoo, the residue was redissolved in CH₂Cl₂ and adsorbed on silica. The residue was purified by flash column chromatography (SiO₂, dry loading on SiO₂, 1-4% MeOH in CH₂Cl₂) to afford the title compound as a light yellow waxy solid (19.5 mg, 72%).

¹H NMR (500 MHz, CD₃OD) δ 8.80 (dd, J=2.5, 0.7 Hz, 1H), 8.53 (td, J=1.9, 0.5 Hz, 1H), 8.29 (ddd, J=8.0, 2.0, 1.1 Hz, 1H), 8.21 (ddd, J=7.8, 1.8, 1.1 Hz, 1H), 7.89 (dd, J=9.6, 2.5 Hz, 1H), 7.75 (td, J=7.9, 0.5 Hz, 1H), 6.45 (dd, J=9.6, 0.7 Hz, 1H), 3.53 (t, J=7.1 Hz, 2H), 3.37 (t, J=6.6 Hz, 2H), 2.51 (td, J=7.1, 2.7 Hz, 2H), 2.33-2.27 (m, 3H), 1.71-1.59 (m, 4H), 1.45 (s, 9H). ¹³C NMR (126 MHz, CD₃OD) δ 174.8, 167.7, 165.7, 161.4, 141.5, 138.2, 136.8, 135.2, 134.9, 133.8, 130.6, 129.7, 122.9, 116.4, 82.1, 81.5, 71.0, 40.7, 40.3, 35.9, 29.8, 28.4, 23.6, 19.7. IR (neat, vₘₐₓ/cm⁻¹) 3299, 3075, 2931, 2483, 1725, 1693, 1635, 1536, 1454, 1368, 1312, 1255, 1191, 1171, 1153, 1085, 1020, 881, 594. HRMS (ESI): m/z=552.1776 [M+Na]⁺ (calc. for C₂₆H₃₁N₃NaO₇S m/z=552.1775)

### Step c) tert-butyl 5-(1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamido)pentanoate

To a vial containing N-(2-(2-azidoethoxy)ethyl)-7-nitrobenzo[c][1,2,5]oxadiazol-4-amine (6.6 mg, 22.7 μmol, 2.0 equiv, CAS RN 2449214-44-0) and tert-butyl 5-(1-((3-(but-3-yn-1-ylcarbamoyl)phenyl)sulfonyl)-6-oxo-1,6-dihydropyridine-3-carboxamido)pentanoate (6.0 mg, 11.3 μmol, 1.0 equiv) was added [Cu(MeCN)₄]PF₆ (21.1 mg, 56.7 μmol, 5.0 equiv) in the glovebox. The mixture was dissolved in argon degassed dry MeOH (0.11 mL) and CH₂Cl₂ (0.11 mL) and the reaction mixture was capped and heated to 40° C. and stirred for 16 h under argon. The reaction mixture was directly loaded on SiO₂ plate and the crude product was purified by preparative TLC (SiO₂, 6-10% MeOH in CH₂Cl₂) and triturated with hexane to afford the title product as an orange solid (8.2 mg, 88%).

¹H NMR (500 MHz, CD₃OD) δ 8.78 (dd, J=2.5, 0.7 Hz, 1H), 8.47 (t, J=1.7 Hz, 1H), 8.44 (d, J=8.9 Hz, 1H), 8.26 (ddd, J=7.9, 2.0, 1.1 Hz, 1H), 8.15 (ddd, J=7.8, 1.7, 1.0 Hz, 1H), 7.85 (dd, J=9.6, 2.5 Hz, 1H), 7.78 (s, 1H), 7.72 (td, J=7.9, 0.5 Hz, 1H), 6.42 (dd, J=9.7, 0.7 Hz, 1H), 6.31 (d, J=8.9 Hz, 1H), 4.54 (t, J=5.2 Hz, 2H), 3.88 (t, J=5.1 Hz, 2H), 3.72 (t, J=5.2 Hz, 2H), 3.65 (bs, 2H), 3.58 (t, J=7.0 Hz, 2H), 3.38-3.33 (m, 2H), 2.89 (t, J=6.9 Hz, 2H), 2.31-2.25 (m, 2H), 1.71-1.56 (m, 4H), 1.44 (s, 9H). ¹³C NMR (126 MHz, CD₃OD) δ174.8, 167.7, 165.7, 161.4, 146.1, 141.5, 138.0, 137.0, 135.2, 134.9, 133.7, 130.6, 129.8, 124.5, 122.9, 116.3, 81.5, 70.3, 70.0, 51.3, 40.7, 35.9, 29.8, 28.4, 26.4, 23.6. IR (neat, vₘₐₓ/cm⁻¹) 3300, 3076, 2925, 2855, 1724, 1693, 1644, 1583, 1532, 1443, 1368, 1301, 1257, 1188, 1150. HRMS (ESI): m/z=845.2641 [M+Na]⁺ (calc. for C₃₆H₄₂N₁₀NaO₁₁S m/z=845.2647)

### Step d) N-(5-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-5-oxopentyl)-1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1, 6-dihydropyridine-3-carboxamide

To a solution of tert-butyl 5-(1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamido)pentanoate (3.8 mg, 4.6 μmol, 1.0 equiv) in dry CH₂Cl₂ (200 μL) was added TFA (100 μL) and the reaction mixture was allowed to stir for 30 min. at rt. Then, the solvents were removed in vacuoo, the residue was re-dissolved in CH₂Cl₂ and co-evaporated (×3). The residue was then dissolved in dry DMF (50 μL). TFA (0.5 M solution in dry DMF, 30.5 μL, 15.2 μmol, 3.3 equiv) and i-Pr₂NEt (0.5 M solution in dry DMF, 49.0 μL, 24.4 μmol, 5.3 equiv) were added at rt. and the resulting solution was cooled to 0° C. Then, HATU (0.1 M solution in DMF, 50.8 μL, 5.0 μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 50.8 μL, 5.0 μmol, 1.1 equiv, Chem. Eur. J., 2020, 26, 1380-1387) was added. The reaction mixture was then allowed to warm up to ambient temperature and stirred for further 35 min. i-Pr₂NEt (0.5 M solution in dry DMF, 2.3 μL, 1.1 μmol, 0.25 equiv) were added at rt. and the resulting solution stirred for additional 10 min. The residue was taken up in CH₂Cl₂ (10 mL) and washed with sat. aq. NH₄Cl(5 mL), sat. aq. NaHCO₃ (5 mL) and brine (5 mL). The organic layer was dried over Na₂SO₄, filtered and concentrated in vacuoo. The crude product was purified by flash column chromatography (SiO₂, 4-5% MeOH in CH₂Cl₂) to afford the product (5.1 mg, 92%) as an orange solid.

¹H NMR (600 MHz, CD₂Cl₂) δ 8.67 (d, J=2.4 Hz, 1H), 8.42 (d, J=8.6 Hz, 1H), 8.37 (s, 1H), 8.19 (d, J=8.0 Hz, 1H), 8.09 (d, J=7.8 Hz, 1H), 7.79 (dd, J=9.7, 2.5 Hz, 1H), 7.64-7.56 (m, 3H), 7.53 (bs, 1H), 6.48 (s, 2H), 6.33 (d, J=9.6 Hz, 1H), 6.21 (d, J=5.4 Hz, 1H), 5.80 (bs, 1H), 5.59 (dt, J=3.1, 1.5 Hz, 1H), 4.51 (t, J=4.9 Hz, 2H), 3.98-3.92 (m, 1H), 3.90-3.85 (m, 3H), 3.84-3.78 (m, 1H), 3.76-3.73 (m, 2H), 3.73-3.72 (m, 2H), 3.71 (s, 6H), 3.66 (bs, 2H), 3.41-3.32 (m, 2H), 3.21 (t, J=7.0 Hz, 2H), 2.95 (t, J=6.4 Hz, 2H), 2.27 (t, J=7.0 Hz, 2H), 2.17-2.11 (m, 1H), 2.06 (t, J=5.7 Hz, 1H), 2.03-1.97 (m, 1H), 1.76-1.49 (m, 8H), 1.32-1.20 (m, 6H), 1.26 (s, 3H), 1.26 (s, 6H), 1.16-1.07 (m, 2H), 0.92 (s, 3H). ¹³C NMR (151 MHz, CD₂Cl₂) δ 173.8, 165.7, 164.0, 160.2, 159.0, 150.1, 145.8, 145.0, 144.7, 140.5, 138.8, 137.3, 137.0, 136.3, 134.2, 134.0, 133.3, 129.9, 128.7, 124.4, 123.3, 122.8, 117.9, 115.8, 103.3, 69.9, 68.9, 56.3, 52.0, 50.7, 47.9, 45.0, 44.9, 44.7, 41.2, 40.2, 40.0, 38.5, 38.0, 36.2, 30.4, 30.2, 29.3, 29.3, 28.7, 28.1, 27.1, 26.6, 25.9, 25.1, 23.2, 21.3. IR (neat, vₘₐₓ/cm⁻¹) 3300, 3075, 2926, 2855, 2096, 1645, 1578, 1532, 1502, 1449, 1411, 1302, 1258, 1189, 1121, 1033. HRMS (ESI): m/z=624.2558 [M+Na₂]²⁺ (calc. for C₅₉H₇₄N₁₄Na₂O₁₂S m/z=624.2558) [α]²⁵_{D}=+26.400±0.321 (c=0.23, CH₂Cl₂).

### Example 2

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide

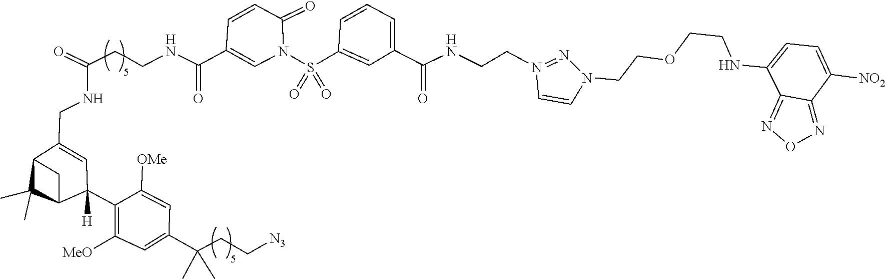

### Step a) tert-butyl 7-(6-hydroxynicotinamido)heptanoate

Solution of 6-hydroxynicotinic acid (346 mg, 2.48 mmol, 1.0 equiv), tert-butyl 7-aminoheptanoate (500 mg, 2.48 mmol, 1.0 equiv, CAS RN 105974-64-9), HOBt·H₂O (476 mg, 3.11 mmol, 1.25 equiv), EDCI·HCl (595 mg, 3.11 mmol, 1.25 equiv) and Et₃N (865 μL, 6.21 mmol, 2.5 equiv) in DMF (8.0 mL) was stirred for 16 h at ambient temperature. The mixture was concentrated in vacuoo and the crude product was purified by flash column chromatography (SiO₂, dry loading on SiO₂, 60% (EtOAc:EtOH, 3:1) in hexanes) to afford the title compound as a white solid (540 mg, 67%).

¹H NMR (500 MHz, CD₂Cl₂) δ 8.04 (dd, J=2.7, 0.7 Hz, 1H), 7.80 (dd, J=9.6, 2.6 Hz, 1H), 6.56 (t, J=5.6 Hz, 1H), 6.49 (dd, J=9.5, 0.7 Hz, 1H), 3.34 (td, J=7.2, 5.7 Hz, 2H), 2.18 (t, J=7.4 Hz, 2H), 1.62-1.51 (m, 4H), 1.42 (s, 9H), 1.40-1.29 (m, 4H). ¹³C NMR (126 MHz, CD₂Cl₂) δ 173.6, 165.2, 164.6, 140.2, 137.4, 119.8, 115.5, 80.4, 40.5, 36.0, 29.9, 29.2, 28.4, 27.2, 25.5. IR (neat, vₘₐₓ/cm⁻¹) 3320, 3070, 2976, 2933, 2861, 1728, 1654, 1635, 1610, 1537, 1461, 1426, 1391, 1366, 1313, 1252, 1219, 1152, 1102. HRMS (ESI): m/z=345.1782 [M+Na]⁺ (calc. for C₁₇H₂₆N₂NaO₄ m/z=345.1785)

### Step b) tert-butyl 7-(6-oxo-1-((3-(prop-2-yn-1-ylcarbamoyl)phenyl)sulfonyl)-1,6-dihydropyridine-3-carboxamido)heptanoate

Tert-butyl 7-(6-hydroxynicotinamido)heptanoate (20.5 mg, 63.5 μmol, 1.0 equiv) was dissolved in anhydrous THF (0.50 mL) at rt. and cooled to −78° C. KO^{t}Bu (1M in THF, 127 μL, 127 μmol, 2.0 equiv) was added dropwise at −78° C. and the solution was stirred for 10 min. at −78° C. Subsequently, 3-(prop-2-yn-1-ylcarbamoyl)benzenesulfonyl chloride (49.2 mg, 191 μmol, 3.0 equiv, CAS RN 1016841-21-6) dissolved in anhydrous THF (0.30 mL) was added dropwise and the mixture was stirred at −78° C. for 2 h before it was allowed to warm up to rt. The solvent was removed in vacuoo, the residue was redissolved in CH₂Cl₂ and adsorbed on silica. The residue was purified by flash column chromatography (SiO₂, dry loading on SiO₂, 30% (EtOAc:EtOH, 3:1) in hexanes) to afford the title compound as a white waxy solid (23.5 mg, 68%).

¹H NMR (500 MHz, CD₃OD) δ 8.80 (dd, J=2.5, 0.7 Hz, 1H), 8.55 (td, J=1.9, 0.5 Hz, 1H), 8.31 (ddd, J=8.0, 2.0, 1.1 Hz, 1H), 8.22 (ddd, J=7.8, 1.7, 1.1 Hz, 1H), 7.89 (dd, J=9.6, 2.5 Hz, 1H), 7.76 (td, J=7.9, 0.5 Hz, 1H), 6.45 (dd, J=9.6, 0.7 Hz, 1H), 4.17 (d, J=2.5 Hz, 2H), 3.36 (t, J=7.2 Hz, 2H), 2.63 (t, J=2.6 Hz, 1H), 2.24 (t, J=7.4 Hz, 2H), 1.68-1.54 (m, 4H), 1.44 (s, 9H), 1.42-1.34 (m, 4H). ¹³C NMR (126 MHz, CD₃OD) δ 175.1, 167.2, 165.7, 161.4, 141.6, 138.2, 136.4, 135.2, 135.1, 134.0, 130.7, 129.8, 122.9, 116.4, 81.4, 80.4, 72.4, 41.1, 36.3, 30.2, 29.8, 28.4, 27.7, 26.1, 23.7. IR (neat, vₘₐₓ/cm⁻¹) 3299, 3073, 2926, 2855, 2471, 2068, 1724, 1691, 1639, 1535, 1453, 1367, 1313, 1259, 1219, 1172, 1151, 1085. HRMS (ESI): m/z=566.1933 [M+Na]⁺ (calc. for C₂₇H₃₃N₃NaO₇S m/z=566.1931)

### Step c) tert-butyl 7-(1-((3-(((1-(2-(2-((7-nitrobenzo[c][1,2,5]oxadiazol-4-yl)amino)ethoxy)ethyl)-1H-1,2, 3-triazol-4-yl)methyl)carbamoyl)phenyl)sulfonyl)-6-oxo-1,6-dihydropyridine-3-carboxamido)heptanoate

To a vial containing N-(2-(2-azidoethoxy)ethyl)-7-nitrobenzo[c][1,2,5]oxadiazol-4-amine (2.5 mg, 8.4 μmol, 1.15 equiv) and tert-butyl 7-(6-oxo-1-((3-(prop-2-yn-1-ylcarbamoyl)phenyl)sulfonyl)-1,6-dihydropyridine-3-carboxamido)heptanoate (4.0 mg, 7.3 μmol, 1.0 equiv) was added [Cu(MeCN)₄]PF₆ (13.7 mg, 36.7 μmol, 5.0 equiv) in the glovebox. The mixture was dissolved in argon degassed CH₂Cl₂ (0.30 mL) and AcOH (10 μL) and was capped and stirred for 16 h under argon. The reaction mixture was directly loaded on SiO₂ plate and the crude product was purified by preparative TLC (SiO₂, 5% MeOH in CH₂Cl₂) to afford the title product as an orange solid (5.3 mg, 86%).

¹H NMR (500 MHz, CD₃OD) δ 8.77 (dd, J=2.5, 0.7 Hz, 1H), 8.53 (td, J=1.9, 0.5 Hz, 1H), 8.42 (d, J=8.8 Hz, 1H), 8.27 (ddd, J=8.0, 2.0, 1.1 Hz, 1H), 8.19 (ddd, J=7.8, 1.7, 1.1 Hz, 1H), 7.92 (s, 1H), 7.85 (dd, J=9.6, 2.5 Hz, 1H), 7.72 (td, J=7.9, 0.5 Hz, 1H), 6.40 (dd, J=9.6, 0.7 Hz, 1H), 6.31 (d, J=8.9 Hz, 1H), 4.60-4.51 (m, 4H), 3.95-3.87 (m, 2H), 3.78-3.71 (m, 2H), 3.66 (bs, 2H), 3.38-3.32 (m, 2H), 2.22 (t, J=7.4 Hz, 2H), 1.69-1.54 (m, 4H), 1.43 (s, 9H), 1.41-1.34 (m, 4H). ¹³C NMR (126 MHz, CD₃OD) δ 175.1, 167.5, 165.7, 161.4, 145.8, 141.5, 138.1, 136.5, 135.2, 135.0, 133.9, 130.6, 129.8, 125.3, 122.9, 116.4, 81.4, 70.3, 70.1, 51.4, 41.1, 36.3, 30.2, 29.8, 28.4, 27.7, 26.1. IR (neat, vₘₐₓ/cm⁻¹) 3300, 3075, 2926, 2855, 1725, 1646, 1583, 1532, 1443, 1367, 1302, 1258, 1189, 1149, 1036. HRMS (ESI): m/z=859.2808 [M+Na]⁺ (calc. for C₃₇H₄₄N₁₀NaO₁₁S m/z=859.2804)

### Step d) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide

To a solution of tert-butyl 7-(1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamido)heptanoate (2.1 mg, 2.6 μmol, 1.0 equiv) in dry CH₂Cl₂ (200 μL) was added TFA (100 μL) and the reaction mixture was allowed to stir for 30 min. at rt. Then, the solvents were removed in vacuoo, the residue was re-dissolved in CH₂Cl₂ and co-evaporated (×3). The residue was then dissolved in dry DMF (50 μL). TFA (0.5 M solution in dry DMF, 17.8 μL, 8.8 μmol, 3.3 equiv) and i-Pr₂NEt (0.5 M solution in dry DMF, 28.5 μL, 14.2 μmol, 5.3 equiv) were added at rt. and the resulting solution was cooled to 0° C. Then, HATU (0.1 M solution in DMF, 29.6 μL, 2.9 μmol, 1.1 equiv) was added, the reaction was stirred at 0° C. for 15 min and amine ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 29.6 μL, 2.9 μmol, 1.1 equiv) was added. The reaction mixture was then allowed to warm up to ambient temperature and stirred for further 45 min. The reaction mixture was then concentrated in vacuoo, the residue taken up in CH₂Cl₂ and directly loaded on a silica plate. Purification by preparative TLC (SiO₂, 4% MeOH in CH₂Cl₂) afforded the product (2.6 mg, 79%) as an orange solid.

¹H NMR (600 MHz, CD₂Cl₂) δ 8.62 (d, J=2.4 Hz, 1H), 8.46 (d, J=8.7 Hz, 1H), 8.43 (s, 1H), 8.26 (d, J=8.1 Hz, 1H), 8.13 (d, J=7.8 Hz, 1H), 7.74 (dd, J=9.7, 2.4 Hz, 1H), 7.72 (s, 1H), 7.63 (t, J=7.9 Hz, 1H), 6.49 (s, 2H), 6.35 (d, J=9.6 Hz, 1H), 6.22 (d, J=8.8 Hz, 1H), 5.58 (s, 1H), 4.65 (d, J=5.5 Hz, 2H), 4.53 (t, J=4.9 Hz, 2H), 3.94 (s, 1H), 3.90 (t, J=5.0 Hz, 2H), 3.86-3.77 (m, 2H), 3.76-3.73 (m, 2H), 3.72 (s, 6H), 3.66 (s, 2H), 3.42-3.34 (m, 2H), 3.22 (t, J=7.0 Hz, 2H), 2.23-1.96 (m, 8H), 1.67 (d, J=8.4 Hz, 1H), 1.64-1.05 (m, 16H), 1.26 (s, 9H), 0.93 (s, 3H). ¹³C NMR (151 MHz, CD₂Cl₂) δ 173.6, 165.5, 163.9, 162.9, 160.1, 159.0, 150.1, 145.1, 144.9, 144.7, 140.5, 139.0, 137.2, 137.1, 135.7, 134.3, 133.7, 133.6, 130.5, 130.3, 129.8, 128.7, 124.2, 122.9, 118.0, 115.8, 103.3, 69.8, 69.0, 56.3, 52.1, 50.8, 47.9, 46.0, 44.9, 44.9, 44.8, 40.3, 38.5, 38.0, 36.8, 36.3, 32.5, 29.9, 29.4, 28.3, 28.1, 27.7, 27.2, 26.6, 26.1, 25.2, 23.3, 21.3. IR (neat, vₘₐₓ/cm⁻¹): 2959, 2924, 2854, 2096, 1649, 1583, 1457, 1411, 1377, 1302, 1260, 1186, 1092, 1018. HRMS (ESI): m/z=1217.5570 [M+H]⁺ (calc. for C₆₀H₇₇N₁₄O₁₂S m/z=1217.5561)

### Example 3

### N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide

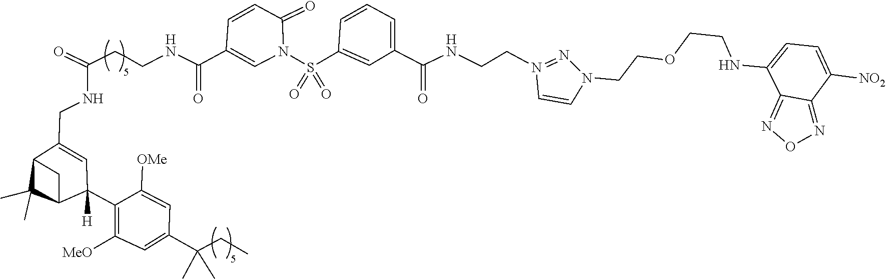

### Step d) N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1, 6-dihydropyridine-3-carboxamide

To a solution of tert-butyl 7-(1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamido)heptanoate (2.5 mg, 3.2 μmol, 1.0 equiv) in dry CH₂Cl₂ (200 μL) was added TFA (100 μL) and the reaction mixture was allowed to stir for 30 min. at rt. Then, the solvents were removed in vacuoo, the residue was re-dissolved in CH₂Cl₂ and co-evaporated (×3). The residue was then dissolved in dry DMF (50 μL). TFA (0.5 M solution in dry DMF, 21.1 μL, 10.5 μmol, 3.3 equiv) and i-Pr₂NEt (0.5 M solution in dry DMF, 33.9 μL, 16.9 μmol, 5.3 equiv) were added at rt. and the resulting solution was cooled to 0° C. Then, HATU (0.1 M solution in DMF, 35.2 μL, 3.5 μmol, 1.1 equiv) was added, the reaction was stirred at 0° C. for 15 min and amine ((1S,4S,5S)-4-(2,6-dimethoxy-4-(2-methyloctan-2-yl)phenyl)-6,6-dimethylbicyclo[3.1.1]hept-2-en-2-yl)methanamine (0.1 M solution in dry CH₂Cl₂, 35.2 μL, 3.5 μmol, 1.1 equiv, Chem. Eur. J, 2020, 26, 1380-1387) was added. The reaction mixture was then allowed to warm up to ambient temperature and stirred for further 45 min. The residue was taken up in CH₂Cl₂ (10 mL) and washed with sat. aq. NH₄Cl(5 mL), sat. aq. NaHCO₃ (5 mL) and brine (5 mL). The organic layer was dried over Na₂SO₄, filtered and concentrated in vacuoo. Purification by preparative TLC (SiO₂, 4% MeOH in CH₂Cl₂) afforded the product (2.7 mg, 72%) as an orange solid.

¹H NMR (500 MHz, CD₂Cl₂) δ 8.61 (dd, J=2.5, 0.7 Hz, 1H), 8.46 (d, J=8.7 Hz, 1H), 8.41 (t, J=1.7 Hz, 1H), 8.25 (ddd, J=8.0, 1.9, 1.1 Hz, 1H), 8.12 (ddd, J=7.8, 1.7, 1.1 Hz, 1H), 7.74 (dd, J=9.7, 2.5 Hz, 1H), 7.70 (s, 1H), 7.63 (td, J=7.9, 0.5 Hz, 1H), 7.44 (s, 1H), 6.71 (t, J=5.7 Hz, 1H), 6.49 (s, 2H), 6.35 (dd, J=9.6, 0.7 Hz, 1H), 6.22 (d, J=8.7 Hz, 1H), 5.58 (dt, J=2.9, 1.5 Hz, 1H), 5.53-5.48 (m, 1H), 4.65 (d, J=5.7 Hz, 2H), 4.57-4.49 (m, 2H), 3.94 (t, J=2.4 Hz, 1H), 3.90 (dd, J=5.4, 4.5 Hz, 2H), 3.88-3.77 (m, 2H), 3.77-3.72 (m, 2H), 3.72 (s, 6H), 3.66 (s, 2H), 3.38 (q, J=6.6 Hz, 2H), 2.24-1.94 (m, 8H), 1.67 (d, J=8.4 Hz, 1H), 1.64-1.05 (m, 16H), 1.26 (s, 9H), 0.93 (s, 3H), 0.85 (t, J=7.7 Hz, 3H). ¹³C NMR (126 MHz, CD₂Cl₂) δ 173.3, 165.5, 163.8, 159.0, 150.2, 140.4, 139.1, 137.2, 135.7, 134.3, 133.5, 129.9, 128.7, 124.2, 122.9, 117.9, 115.8, 103.3, 69.8, 69.0, 56.3, 50.8, 48.0, 44.9, 44.9, 44.8, 41.2, 40.3, 38.5, 38.0, 37.0, 36.1, 32.3, 30.6, 29.9, 29.5, 29.3, 28.8, 28.1, 26.6, 26.6, 26.0, 25.2, 23.2, 21.3, 14.4. IR (neat, vₘₐₓ/cm⁻¹): 3311, 3074, 2957, 2924, 2853, 1736, 1645, 1575, 1532, 1458, 1411, 1377, 1301, 1260, 1189, 1116, 1022. HRMS (ESI): m/z=1198.5360 [M+Na]⁺ (calc. for C₆₀H₇₇NNNaO₁₂S m/z=1198.5366)

### Example 4

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamide

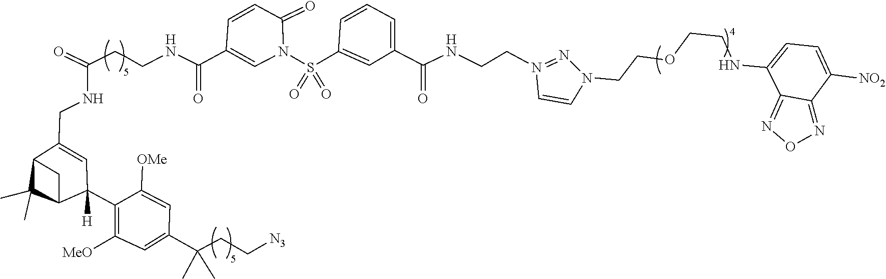

### Step c) tert-butyl 7-(1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamido)heptanoate

To a heart-shaped flask loaded with tert-butyl 7-(6-oxo-1-((3-(prop-2-yn-1-ylcarbamoyl)phenyl)sulfonyl)-1,6-dihydropyridine-3-carboxamido)heptanoate (5.8 mg, 10.7 μmol, 1.3 equiv), N-(14-azido-3,6,9,12-tetraoxatetradecyl)-7-nitrobenzo[c][1,2,5]oxadiazol-4-amine (3.5 mg, 8.2 μmol, 1.0 equiv) and Cu[MeCN]₄PF₆ (15.3 mg, 41.1 μmol, 5.0 equiv) was added dry CH₂Cl₂ (0.3 mL, previously degassed with argon) and 20 μL of AcOH. The flask was then flushed with argon, stoppered with a glass stopper and the resulting deep orange solution was allowed to stir for 16 h. The reaction mixture was then directly loaded onto a silica plate and purified by preparative TLC (SiO₂, 5% MeOH in CH₂Cl₂) to afford the product (7.2 mg, 90%) as an orange solid.

¹H NMR (500 MHz, CD₃OD) δ=8.76 (dd, J=2.5, 0.7 Hz, 1H), 8.56 (d, J=1.8 Hz, 1H), 8.47 (d, J=8.9 Hz, 1H), 8.27 (ddd, J=8.1, 1.9, 1.0 Hz, 1H), 8.21 (d, J=7.8 Hz, 1H), 8.01 (s, 1H), 7.86 (dd, J=9.6, 2.5 Hz, 1H), 7.73 (t, J=7.9 Hz, 1H), 6.42 (dd, J=9.6, 0.7 Hz, 1H), 6.39 (d, J=8.9 Hz, 1H), 4.64 (t, J=5.0 Hz, 2H), 4.57-4.50 (m, 2H), 3.84 (t, J=4.6 Hz, 2H), 3.79 (t, J=5.5 Hz, 2H), 3.71 (bs, 1H), 3.65-3.61 (m, 1H), 3.60-3.57 (m, 2H), 3.56-3.52 (m, 2H), 3.52-3.49 (m, 4H), 3.48-3.43 (m, 2H), 3.38-3.33 (m, 2H), 3.32-3.28 (2H, HSQC), 2.23 (t, J=7.4 Hz, 2H), 1.61 (td, J=10.0, 7.0 Hz, 4H), 1.44 (s, 9H), 1.39 (tt, J=5.2, 2.3 Hz, 4H). ¹³C NMR (126 MHz, CD₃OD) δ=175.08, 167.38, 165.63, 161.36, 141.49, 138.12, 135.19, 135.09, 133.90, 130.64, 129.80, 122.94, 116.35, 81.37, 71.61, 71.56, 71.54, 71.44, 71.42, 70.31, 51.49, 41.09, 36.34, 30.24, 29.82, 28.36, 27.74, 26.11. IR (neat, vₘₐₓ/cm⁻¹) 3300, 3073, 2924, 2857, 1724, 1691, 1651, 1621, 1583, 1531, 1499, 1443, 1367, 1299, 1258, 1188, 1144, 1094, 1031. HRMS (ESI): m/z=969.3757 [M+H]⁺ (calc. for C₄₃H₅₇N₁₀O₁₄S m/z=969.3771)

### Step d) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(14-((7-nitrobenzo[c][1,2,5]oxadiazol-4-yl)amino)-3,6,9,12-tetraoxatetradecyl)-1H-1, 2,3-triazol-4-yl)methyl)carbamoyl)phenyl)sulfonyl)-6-oxo-1, 6-dihydropyridine-3-carboxamide

To a solution of tert-butyl 7-(1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamido)heptanoate (7.2 mg, 7.4 μmol, 1.0 equiv) in dry CH₂Cl₂ (200 μL) was added TFA (100 μL) and the resulting orange reaction mixture was allowed to stir for 30 min. Then, the solvents were concentrated in vacuoo, the residue was redissolved in CH₂Cl₂ and co-evaporated (×3). The residue was then dissolved in dry DMF (0.1 mL). TFA (0.5 M solution in dry DMF, 49.0 μL, 24.5 μmol, 3.3 equiv) and i-Pr₂NEt (0.5 M solution in dry DMF, 78.8 μL, 39.4 μmol, 5.3 equiv) were added and the resulting light orange solution was cooled to 0° C. Then, HATU (0.1 M solution in DMF, 81.7 μL, 8.2 μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 81.7 μL, 8.2 μmol, 1.1 equiv) was added. The reaction mixture was then allowed to warm to ambient temperature and stirred for further 30 min. Reaction control by LCMS indicated incomplete conversion and more i-Pr₂NEt (0.5 M solution in dry DMF, 7.4 μL, 3.7 μmol, 0.5 equiv) was added. The reaction mixture was then concentrated in vacuoo, the residue taken up in CH₂Cl₂ and directly loaded onto a silica plate. Purification by preparative TLC (SiO₂, 7% MeOH in CH₂Cl₂) afforded the product (5.7 mg, 57%) as an orange solid.

¹H NMR (500 MHz, CD₂Cl₂) 6=8.63 (d, J=2.4 Hz, 1H), 8.57 (s, 1H), 8.45 (d, J=8.7 Hz, 1H), 8.34 (d, J=7.9 Hz, 1H), 8.29 (s, 1H), 8.21 (s, 1H), 8.19 (d, J=7.9 Hz, 1H), 7.75 (dd, J=9.6, 2.4 Hz, 1H), 7.64 (t, J=7.9 Hz, 1H), 7.36 (s, 1H), 7.23 (s, 1H), 6.49 (s, 2H), 6.36 (d, J=9.5 Hz, 1H), 6.21 (d, J=8.7 Hz, 1H), 5.97 (s, 1H), 5.64-5.59 (m, 1H), 4.80 (d, J=5.2 Hz, 2H), 4.59 (t, J=4.5 Hz, 2H), 3.95 (s, 1H), 3.86 (dt, J=12.1, 5.1 Hz, 4H), 3.84-3.80 (m, 2H), 3.72 (s, 6H), 3.70-3.65 (m, 4H), 3.65-3.61 (m, 2H), 3.60-3.56 (m, 4H), 3.54-3.49 (m, 4H), 3.37 (q, J=6.4 Hz, 2H), 3.22 (t, J=7.0 Hz, 2H), 2.29 (t, J=7.6 Hz, 2H), 2.22-2.13 (m, 1H), 2.07 (t, J=5.5 Hz, 1H), 2.02 (d, J=5.8 Hz, 1H), 1.67 (d, J=8.4 Hz, 1H), 1.64-1.50 (m, 8H), 1.40-1.20 (m, 8H), 1.27 (s, 3H), 1.26 (s, 6H), 1.15-1.07 (m, 2H), 0.94 (s, 3H). ¹³C NMR (126 MHz, CD₂Cl₂) 6=173.55, 166.05, 165.20, 164.06, 160.22, 159.00, 150.05, 145.03, 140.82, 139.01, 137.28, 137.12, 135.48, 134.74, 133.87, 133.68, 129.67, 128.49, 124.22, 122.82, 117.94, 115.96, 103.29, 71.12, 71.04, 70.91, 70.81, 69.43, 56.26, 52.06, 51.78, 47.95, 44.94, 44.88, 44.77, 41.23, 40.26, 38.49, 38.03, 37.02, 35.23, 32.50, 30.39, 30.26, 29.51, 29.36, 29.28, 28.95, 28.11, 27.74, 27.15, 26.76, 26.57, 26.11, 25.15, 23.26, 21.30. IR (neat, vₘₐₓ/cm⁻¹) 3289, 3074, 2932, 2863, 2096, 1648, 1579, 1531, 1448, 1410, 1380, 1349, 1302,1257, 1189, 1121, 1036. HRMS (ESI): m/z=1349.6341 [M+H]⁺ (calc. for C₆₆H₈₉N₁₄O₁₅S m/z=1349.6347)

### Example 5

### 1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

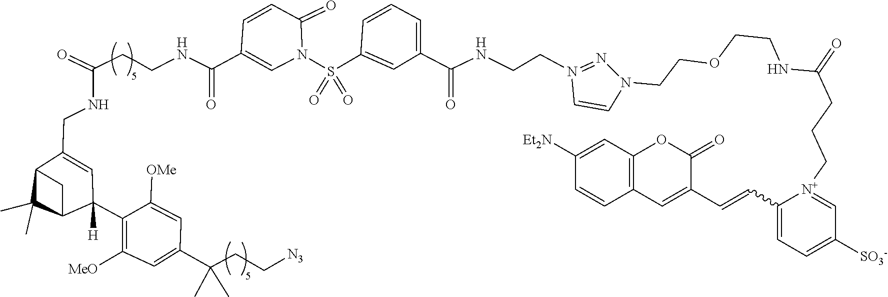

### Step c) 1-(6-((2-(2-(4-((3-((5-((7-(tert-butoxy)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

To a flask loaded with tert-butyl 7-(6-oxo-1-((3-(prop-2-yn-1-ylcarbamoyl)phenyl)sulfonyl)-1,6-dihydropyridine-3-carboxamido)heptanoate (3.9 mg, 7.1 μmol, 1.5 equiv) and 1-(6-((2-(2-azidoethoxy)ethyl)amino)-6-oxohexyl)-6-(2-(7-(diethylamino)-2-oxo-2H-chromen-3-yl)vinyl)pyridin-1-ium-3-sulfonate (3.0 mg, 4.7 μmol, 1.0 equiv) was added [Cu(MeCN)₄]PF₆ (8.9 mg, 23.9 μmol, 5.0 equiv) in the glovebox. The solids were dissolved in argon degassed, dry CH₂Cl₂ (0.30 mL) and AcOH (10 μL). The flask was then flushed again with argon, stoppered and stirred for 16 h at rt. The reaction mixture was then directly loaded onto a SiO₂ plate and purified by preparative TLC (SiO₂, 15% MeOH in CH₂Cl₂) to afford the product as a red solid (4.6 mg, 82%).

¹H NMR (400 MHz, CD₃OD, E/Z signal sets reported as full signals) δ=9.20 (d, J=1.8 Hz, 1H, Z), 9.06 (d, J=1.9 Hz, 1H, E), 8.76 (dd, J=2.5, 0.7 Hz, 1H), 8.57 (dd, J=8.3, 1.8 Hz, 1H, E), 8.55 (s, 1H), 8.51 (dd, J=8.3, 1.8 Hz, 1H, Z), 8.42 (d, J=8.7 Hz, 1H, E), 8.29-8.22 (m, 1H), 8.19 (dt, J=7.9, 1.3 Hz, 1H), 8.12 (s, 1H, E), 8.09 (d, J=15.4 Hz, 1H, E), 7.96 (s, 1H), 7.91 (s, 1H, Z), 7.89 (d, J=8.4 Hz, 1H, Z), 7.85 (dd, J=9.7, 2.4 Hz, 1H), 7.79 (d, J=15.3 Hz, 1H, E), 7.71 (t, J=7.9 Hz, 1H), 7.48 (d, J=9.1 Hz, 1H, E), 7.36 (d, J=9.0 Hz, 1H, Z), 7.09 (dd, J=12.3, 0.9 Hz, 1H, Z), 6.81 (dd, J=9.0, 2.5 Hz, 1H, E), 6.79 (dd, J=12.3, 0.9 Hz, 1H, Z), 6.72 (dd, J=9.0, 2.5 Hz, 1H, Z), 6.52 (d, J=2.4 Hz, 1H, E), 6.44 (d, J=2.4 Hz, 1H, Z), 6.40 (dd, J=9.6, 0.8 Hz, 1H), 4.69 (t, J=7.8 Hz, 2H), 4.61 (s, 2H), 4.55 (t, J=4.9 Hz, 2H), 3.86-3.78 (m, 2H), 3.55 (t, J=7.2 Hz, 2H), 3.52-3.45 (m, 4H), 3.36-3.26 (m, 4H), 2.22 (t, J=7.3 Hz, 2H), 2.20-2.15 (m, 2H), 2.10-1.92 (m, 4H), 1.73-1.53 (m, 6H), 1.51-1.46 (m, 2H), 1.43 (s, 9H), 1.40-1.35 (m, 2H), 1.24 (t, J=7.1 Hz, 3H), 1.20 (t, J=7.1 Hz, 3H). ¹³C NMR (101 MHz, CD₃OD) δ=168.9, 161.3, 158.3, 155.4, 154.5, 149.2, 145.9, 143.8, 142.8, 141.5, 135.1, 132.3, 125.4, 116.4, 114.7, 97.6, 81.4, 70.5, 70.1, 51.5, 46.1, 41.1, 40.2, 36.3, 30.8, 30.2, 29.8, 28.4, 27.8, 26.7, 26.1, 12.8.

IR (neat, vₘₐₓ/cm⁻¹) 3311, 3064, 2956, 2924, 2854, 1718, 1657, 1618, 1579, 1556, 1504, 1457, 1419, 1377, 1356, 1259, 1191, 1170, 1134, 1081, 1040, 1016. HRMS (ESI): m/z=1170.4638 [M+H]⁺ (calc. for C₅₇H₇₂N₉O₁₄S₂ m/z=1170.4635)

### Step d) 1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

To a solution of ester 1-(6-((2-(2-(4-((3-((5-((7-(tert-butoxy)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate (4.4 mg, 3.7 μmol, 1.0 equiv) in dry CH₂Cl₂ (200 μL) was added TFA (100 μL) and the resulting deep red reaction mixture was allowed to stir for 30 min. at rt. Then, the solvents were removed in vacuoo, the residue was re-dissolved in CH₂Cl₂ and co-evaporated (×3). The residue was then dissolved in dry DMF (75 μL). TFA (0.5 M solution in dry DMF, 24.8 μL, 12.4 μmol, 3.3 equiv) and i-Pr₂NEt (0.5 M solution in dry DMF, 39.9 μL, 19.9 μmol, 5.3 equiv) were added at rt. and the resulting light red solution was cooled to 0° C. Then, HATU (0.1 M solution in DMF, 41.4 μL, 4.1 μmol, 1.1 equiv) was added, the reaction was stirred at 0° C. for 15 min and amine ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 41.4 μL, 4.1 μmol, 1.1 equiv) was added. The reaction mixture was then allowed to warm up to ambient temperature and stirred for further 30 min. Reaction control by LCMS indicated incomplete conversion and more i-Pr₂NEt (0.5 M solution in dry DMF, 3.8 μL, 1.9 μmol, 0.5 equiv) was added and the mixture was stirred for additional 5 min. The reaction mixture was then concentrated in vacuoo, the residue taken up in CH₂Cl₂ and directly loaded on a silica plate. Purification by preparative TLC (SiO₂, 10% MeOH in CH₂Cl₂) afforded the product as a red solid (4.4 mg, 75%).

¹H NMR (600 MHz, CD₂Cl₂, E/Z signal sets reported as full signals) 6=9.64 (s, 1H, Z), 9.59 (s, 1H, E), 9.01 (bs, 1H, E), 8.96 (bs, 1H, Z), 8.78-8.75 (m, 1H), 8.71 (t, J=1.7 Hz, 1H), 8.69 (d, J=9.2 Hz, 1H, E), 8.60 (dd, J=8.3, 1.7 Hz, 1H, Z), 8.51-8.44 (m, 1H), 8.36 (ddd, J=6.2, 3.1, 1.6 Hz, 1H), 8.26 (d, J=8.5 Hz, 1H, E), 8.13 (d, J=15.3 Hz, 1H, E), 8.09 (bs, 1H, E), 8.02 (bs, 1H, Z), 7.93 (s, 1H), 7.87 (dt, J=9.7, 2.7 Hz, 1H), 7.81 (s, 1H, Z), 7.80 (s, 1H, E), 7.71 (d, J=8.3 Hz, 1H, Z), 7.69-7.66 (m, 1H), 7.63 (d, J=15.3 Hz, 1H, E), 7.40 (d, J=9.0 Hz, 1H, E), 7.26 (d, J=9.0 Hz, 1H, E), 7.01 (dd, J=12.3, 0.9 Hz, 1H, Z), 6.68 (dd, J=9.0, 2.4 Hz, 1H, E), 6.62 (dd, J=9.0, 2.5 Hz, 1H, Z), 6.53 (d, J=2.8 Hz, 1H, E), 6.52 (d, J=7.1 Hz, 1H, Z), 6.48 (s, 2H, Z), 6.48 (s, 2H, E), 6.43 (d, J=2.4 Hz, 1H, Z), 6.35 (dd, J=9.9, 2.8 Hz, 1H), 6.32 (bt, J=6.3 Hz, 1H, Z), 6.25 (t, J=5.4 Hz, 1H, E), 5.59-5.56 (m, 1H, E), 5.56-5.53 (m, 1H, Z), 4.78 (t, J=7.7 Hz, 2H, E), 4.72 (t, J=7.8 Hz, 2H, Z), 4.64 (dd, J=5.7, 3.3 Hz, 2H), 4.50-4.44 (m, 2H), 3.94-3.91 (m, 1H), 3.86-3.80 (m, 2H), 3.80-3.74 (m, 2H), 3.72 (s, 6H, Z), 3.71 (s, 6H, E), 3.57-3.51 (m, 2H), 3.48 (q, J=7.2 Hz, 2H, E), 3.44 (q, J=7.1 Hz, 2H, Z), 3.34 (dt, J=12.3, 6.4 Hz, 2H), 3.29 (d, J=5.6 Hz, 2H), 3.21 (t, J=7.0 Hz, 2H), 2.21-2.16 (m, 2H), 2.13 (dt, J=8.4, 5.5 Hz, 1H), 2.06 (t, J=6.6 Hz, 2H), 2.03-1.94 (m, 3H), 1.91 (td, J=7.2, 3.0 Hz, 1H), 1.69-1.48 (m, 1OH), 1.42-1.38 (m, 4H), 1.33-1.30 (m, 6H), 1.29-1.27 (m, 2H), 1.26 (s, 9H), 1.25-1.23 (m, 3H, E), 1.20 (t, J=7.1 Hz, 3H, Z), 1.14-1.02 (m, 2H), 0.92 (s, 3H, E), 0.92 (s, 3H, Z). ¹³C NMR (151 MHz, CD₂Cl₂) δ 175.6, 173.4, 173.3, 173.2, 173.2, 165.2, 165.1, 164.1, 164.1, 160.6, 160.4, 159.0, 157.7, 157.6, 154.6, 153.8, 153.6, 153.0, 150.0, 150.0, 148.3, 146.5, 145.8, 145.8, 145.8, 144.4, 143.6, 143.3, 142.2, 142.0, 141.9, 141.9, 139.3, 139.1, 137.2, 137.1, 136.7, 135.6, 135.5, 135.4, 135.3, 135.1, 134.9, 133.2, 133.2, 131.7, 131.3, 130.7, 130.5, 130.3, 129.4, 128.5, 128.3, 127.4, 127.3, 125.0, 124.9, 124.8, 124.1, 123.9, 122.5, 118.1, 118.0, 117.7, 116.0, 115.9, 114.4, 114.0, 110.8, 110.4, 109.5, 108.7, 103.3, 97.4, 97.3, 69.7, 69.6, 69.6, 69.4, 66.0, 59.1, 58.7, 56.3, 52.1, 50.8, 50.8, 48.0, 45.8, 45.6, 44.9, 44.9, 44.8, 44.8, 44.7, 41.2, 40.5, 40.4, 40.1, 38.5, 38.0, 37.6, 37.3, 37.1, 36.3, 36.1, 36.1, 36.0, 32.5, 31.7, 31.2, 30.6, 30.3, 30.3, 30.1, 29.9, 29.9, 29.8, 29.8, 29.7, 29.5, 29.4, 29.3, 28.1, 27.7, 27.7, 27.3, 27.2, 27.1, 26.6, 26.4, 26.2, 26.1, 26.0, 25.1, 25.0, 24.9, 23.3, 14.4, 12.8, 12.8. IR (neat, vₘₐₓ/cm⁻¹) 3358, 2956, 2923, 2853, 2096, 1632, 1578, 1555, 1504, 1459, 1420, 1378, 1378, 1355, 1321, 1259, 1238, 1191,1134, 1121, 1080, 1041. HRMS (ESI): m/z=775.8653 [M+H]²⁺ (calc. for C₈₀H₁₀₅N₁₃O₅S₂ m/z=775.8642)

### Example 6

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide

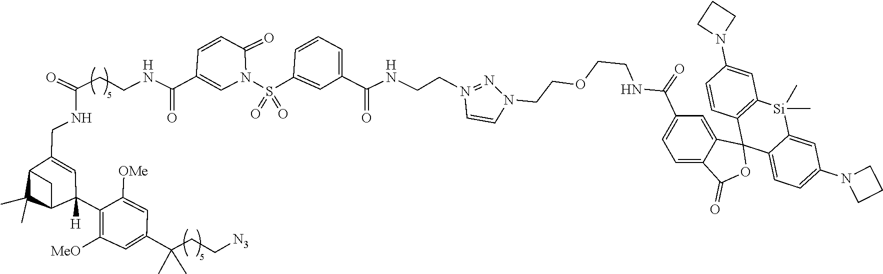

### 3, 7-di(azetidin-1-yl)-N-(2-(2-azidoethoxy)ethyl)-5,5-dimethyl-3′-oxo-3′H,5H-spiro[dibenzo[b,e]siline-10,1′-isobenzofuran]-6′-carboxamide

To a solution of tert-butyl 3,7-di(azetidin-1-yl)-5,5-dimethyl-3′-oxo-3′H,5H-spiro[dibenzo[b,e]siline-10,1′-isobenzofuran]-6′-carboxylate (9.5 mg, 17.2 μmol, 1.0 equiv, prepared as described in Nat. Methods, 2020, 17, 815-821) in dry CH₂Cl₂ (200 μL) was added TFA (100 μL) and the resulting deep blue solution was allowed to stir for 1 h. Then, the solvents were evaporated in vacuoo, the residue was redissolved in CH₂Cl₂ and co-evaporated (×3). To the crude product was then added i-Pr₂NEt (0.5 solution in DMF, 172 μL, 86.4 μmol, 5.0 equiv) and the resulting light blue solution was cooled to 0° C. Then, HATU (0.1 M solution in DMF, 258 μL, 25.8 μmol, 1.5 equiv) was added, the reaction was stirred at 0° C. for 15 min, 2-(2-Azidoethoxy)ethanamine (2.4 mg, 5.3 μmol, 1.0 equiv, CAS RN 464190-91-8) in CH₂Cl₂ (0.2 mL) was added at 0° C., the mixture was allowed to warm to ambient temperature and stirred for further 30 min. The reaction mixture was then diluted with sat. aq. NaHCO₃ (5 mL) and CH₂Cl₂ (10 mL) and the phases were separated. The aqueous phase was extracted with CH₂Cl₂ (2×10 mL), dried over Na₂SO₄ and concentrated in vacuoo. Purification by preparative TLC (SiO₂, 3% MeOH in CH₂Cl₂) afforded the product (6.0 mg, 57%) as a blue solid.

¹H NMR (500 MHz, CD₂Cl₂) 6=7.96 (dd, J=8.0, 0.8 Hz, 1H), 7.89 (dd, J=8.0, 1.4 Hz, 1H), 7.60 (dd, J=1.4, 0.8 Hz, 1H), 6.73 (d, J=8.7 Hz, 2H), 6.68 (d, J=2.6 Hz, 2H), 6.56 (s, 1H), 6.28 (dd, J=8.7, 2.6 Hz, 2H), 3.89 (t, J=7.3 Hz, 8H), 3.69-3.63 (m, 2H), 3.61 (dtd, J=4.8, 3.6, 1.3 Hz, 4H), 3.37-3.32 (m, 2H), 2.36 (tt, J=7.8, 6.8 Hz, 4H), 0.62 (s, 3H), 0.54 (s, 3H). ¹³C NMR (126 MHz, CD₂Cl₂) 6=170.16, 166.50, 155.83, 151.77, 140.60, 136.85, 132.39, 129.16, 128.37, 127.96, 126.30, 123.57, 116.08, 112.92, 70.68, 70.10, 52.85, 51.20, 40.46, 17.43, 0.41, -0.97. IR (neat, vₘₐₓ/cm⁻¹) 3332, 2924, 2854, 2103, 1755, 1662, 1594, 1547, 1478, 1405, 1352, 1306, 1267, 1246, 1163, 1124, 1097, 1039. HRMS (ESI): m/z=609.2641 [M+H]⁺ (calc. for C₃₃H₃₇N₆O₄Si m/z=609.2640)

### Step c) tert-butyl 7-(1-((3-(((1-(2-(2-(3, 7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2, 3-triazol-4-yl)methyl)carbamoyl)phenyl)sulfonyl)-6-oxo-1, 6-dihydropyridine-3-carboxamido)heptanoate

To a heart-shaped flask loaded with tert-butyl 7-(6-oxo-1-((3-(prop-2-yn-1-ylcarbamoyl)phenyl)sulfonyl)-1,6-dihydropyridine-3-carboxamido)heptanoate (2.0 mg, 3.7 μmol, 1.5 equiv), 3,7-di(azetidin-1-yl)-N-(2-(2-azidoethoxy)ethyl)-5,5-dimethyl-3′-oxo-3′H,5H-spiro[dibenzo[b,e]siline-10,1′-isobenzofuran]-6′-carboxamide (1.5 mg, 2.5 μmol, 1.0 equiv) and Cu[MeCN]₄PF₆ (4.6 mg, 12.3 μmol, 5.0 equiv) was added dry CH₂Cl₂ (0.3 mL, previously degassed with argon) and 10 μL of AcOH. The flask was then flushed with argon, stoppered with a glass stopper and the resulting deep blue solution was allowed to stir for 16 h. The reaction mixture was then directly loaded onto a silica plate and purified by preparative TLC (SiO₂, 6% MeOH in CH₂Cl₂) to afford the product (1.4 mg, 49%) as a blue solid.

¹H NMR (500 MHz, CD₃OD) δ=8.77 (dd, J=2.5, 0.7 Hz, 1H), 8.53 (td, J=1.9, 0.5 Hz, 1H), 8.26 (ddd, J=8.0, 2.0, 1.1 Hz, 1H), 8.15 (ddd, J=7.9, 1.7, 1.1 Hz, 1H), 8.01-7.92 (m, 2H), 7.88 (s, 1H), 7.82 (dd, J=9.7, 2.5 Hz, 1H), 7.71-7.64 (m, 2H), 6.71 (dd, J=2.7, 0.4 Hz, 2H), 6.69 (dd, J=8.7, 0.4 Hz, 2H), 6.37 (dd, J=9.6, 0.7 Hz, 1H), 6.29 (dd, J=8.7, 2.7 Hz, 2H), 4.52 (t, J=4.8 Hz, 2H), 4.37 (s, 2H), 3.85 (t, J=7.2 Hz, 8H), 3.82 (t, J=4.3 Hz, 2H), 3.56 (t, J=5.4 Hz, 3H), 3.48 (t, J=5.3 Hz, 3H), 3.34-3.32 (m, 2H), 2.34 (p, J=7.3 Hz, 4H), 2.22 (t, J=7.4 Hz, 2H), 1.66-1.53 (m, 4H), 1.43 (s, 9H), 1.40-1.35 (m, 4H), 0.59 (s, 3H), 0.51 (s, 3H). ¹³C NMR (126 MHz, CD₃OD) δ=196.21 (HMBC), 161.31 (HSQC), 152.79, 141.44, 138.10, 136.51, 135.18, 133.91, 133.28, 129.01, 126.75, 125.43, 124.67, 122.91, 116.89, 113.86, 70.15, 53.36, 51.42, 36.33, 30.23, 29.81, 28.37, 27.74, 26.11, 24.08, 17.77, 17.28, 10.35, 0.26, -1.31. IR (neat, vₘₐₓ/cm⁻¹) 3310, 2928, 2856, 1756, 1654, 1594, 1545, 1454, 1367, 1306, 1247, 1189, 1151, 1058. HRMS (ESI): m/z=1152.4678 [M+H]⁺ (calc. for C₆₀H₇₀N₉O₁₁SSi m/z=1152.4679)

### Step d) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3, 7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1, 6-dihydropyridine-3-carboxamide

To a solution of tert-butyl 7-(1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamido)heptanoate (4.0 mg, 3.5 μmol, 1.0 equiv) in dry CH₂Cl₂ (200 μL) was added TFA (100 μL) and the resulting deep blue reaction mixture was allowed to stir for 1 h. Then, the solvents were evaporated in vacuoo, the residue was redissolved in CH₂Cl₂ and co-evaporated (×3). The residue was then dissolved in dry DMF (0.1 mL). TFA (0.3 M solution in dry DMF, 38.2 μL, 11.5 μmol, 3.3 equiv) and i-Pr₂NEt (0.5 M solution in dry DMF, 43.7 μL, 21.9 μmol, 6.3 equiv) were added and the resulting light green solution was cooled to 0° C. Then, HATU (0.1 M solution in DMF, 38.2 μL, 3.8 μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine was added (0.05 M solution in dry CH₂Cl₂, 72.8 μL, 3.6 μmol, 1.05 equiv). The reaction mixture was then allowed to warm to ambient temperature and stirred for further 30 min. The reaction mixture was then concentrated in vacuoo, the residue taken up in CH₂Cl₂ and directly loaded onto a silica plate. Purification by preparative TLC (SiO₂, 10% MeOH in CH₂Cl₂) afforded the product (2.2 mg, 41%) as a blue solid.

¹H NMR (600 MHz, CDCl₃) δ=8.66 (s, 1H), 8.50 (s, 1H), 8.37 (d, J=8.3 Hz, 1H), 8.12 (d, J=7.7 Hz, 1H), 8.01 (d, J=7.9 Hz, 1H), 7.93 (d, J=7.9 Hz, 1H), 7.74 (s, 2H), 7.67 (s, 1H), 7.62 (t, J=7.9 Hz, 1H), 6.78 (d, J=8.7 Hz, 2H), 6.64 (d, J=2.6 Hz, 2H), 6.47 (s, 2H), 6.35 (d, J=9.6 Hz, 1H), 6.24 (dd, J=8.7, 2.7 Hz, 2H), 5.61 (s, 1H), 4.56-4.35 (m, 4H), 3.96 (s, 1H), 3.93-3.87 (m, 8H), 3.85-3.83 (m, 2H), 3.83-3.77 (m, 2H), 3.73 (s, 6H), 3.58 (dd, J=9.2, 4.9 Hz, 2H), 3.32-3.27 (m, 1H), 3.22 (t, J=7.0 Hz, 1H), 2.36 (p, J=15.8, 7.5 Hz, 4H), 2.22 (t, J=9.8 Hz, 2H), 2.19-2.14 (m, 1H), 2.07-2.04 (m, 1H), 2.01 (q, J=6.3 Hz, 2H), 1.96 (s, 1H), 1.77-1.46 (m, 9H), 1.38-1.16 (m, 17H), 1.16-0.99 (m, 2H), 0.94 (s, 3H). ¹³C NMR (151 MHz, CDCl₃) δ=175.43, 158.39, 127.92, 115.82, 113.94, 112.50, 102.69, 55.81, 52.29, 51.46, 47.23, 44.42, 37.11, 35.91, 31.93, 27.18, 25.52, 22.69, 14.13. IR (neat, vₘₐₓ/cm⁻¹) 3358, 2923, 2853, 2096, 1742, 1658, 1634, 1595, 1537, 1467, 1411, 1377, 1259, 1187, 1092, 1016. HRMS (ESI): m/z=1532.7252 [M+H]⁺ (calc. for C₈₃H₁₀₂N₁₃O₁₂SSi m/z=1532.7255)

### Example 7

### N-(9-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-9-oxononyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide

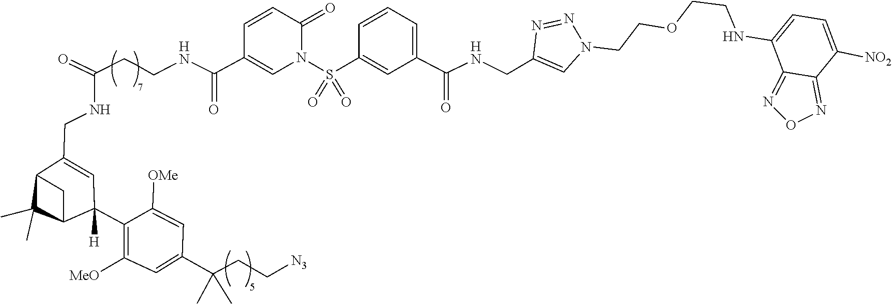

### Step a) tert-butyl 9-(6-hydroxynicotinamido)nonanoate

Solution of 6-hydroxynicotinic acid (91.0 mg, 654 μmol, 1.0 equiv), tert-butyl 9-aminononanoate (150 mg, 654 μmol, 1.0 equiv, CAS RN 134857-22-0), HOBt·H₂O (125 mg, 818 μmol, 1.25 equiv), EDCI·HCl (157 mg, 818 μmol, 1.25 equiv) and Et₃N (228 μL, 1.64 mmol, 2.5 equiv) in DMF (1.5 mL) was stirred for 16 h at ambient temperature. The mixture was concentrated in vacuoo and the crude product was purified by flash column chromatography (SiO₂, dry loading on SiO₂, 50% (EtOAc:EtOH, 3:1) in hexanes) to afford the title compound as a white solid (162 mg, 71%).

¹H NMR (400 MHz, CD₂Cl₂) δ 12.64 (bs, 1H), 8.09 (d, J=2.6 Hz, 1H), 7.84 (dd, J=9.5, 2.5 Hz, 1H), 7.11 (t, J=5.6 Hz, 1H), 6.45 (d, J=9.5 Hz, 1H), 3.39-3.26 (m, 2H), 2.16 (t, J=7.5 Hz, 2H), 1.65-1.46 (m, 4H), 1.41 (s, 9H), 1.37-1.23 (m, 8H). ¹³C NMR (101 MHz, CD₂Cl₂) δ 173.7, 165.2, 164.7, 140.3, 137.4, 119.7, 115.5, 80.3, 40.6, 36.1, 30.1, 29.8, 29.7, 29.6, 28.4, 27.5, 25.6. IR (neat, vₘₐₓ/cm⁻¹) 3327, 2930, 2853, 2700, 1727, 1690, 1625, 1608, 1531, 1474, 1432, 1420, 1390, 1365, 1312, 1254, 1154, 1102. HRMS (ESI): m/z=373.2098 [M+Na]⁺ (calc. for C₁₉H₃₀N₂NaO₄ m/z=373.2098)

### Step b) tert-butyl 9-(6-oxo-1-((3-(prop-2-yn-1-ylcarbamoyl)phenyl)sulfonyl)-1,6-dihydropyridine-3-carboxamido)nonanoate

Tert-butyl 9-(6-hydroxynicotinamido)nonanoate (20.0 mg, 57.0 μmol, 1.0 equiv) was dissolved in anhydrous THF (0.50 mL) at rt. with the aid of sonication and cooled to −78° C. KO^{t}Bu (1M in THF, 114 μL, 114 μmol, 2.0 equiv) was added dropwise at −78° C. and the solution was stirred for 10 min. at −78° C. Subsequently, 3-(prop-2-yn-1-ylcarbamoyl)benzenesulfonyl chloride (44.1 mg, 171 μmol, 3.0 equiv, CAS RN 1016841-21-6) dissolved in anhydrous THF (0.30 mL) was added dropwise and the mixture was stirred at −78° C. for 2 h before it was allowed to warm up to rt. The solvent was removed in vacuoo, the residue was redissolved in CH₂Cl₂ and adsorbed on silica. The residue was purified by flash column chromatography (SiO₂, dry loading on SiO₂, 30% (EtOAc:EtOH, 3:1) in hexanes) to afford the title compound as a white waxy solid (23 mg, 70%).

¹H NMR (400 MHz, CD₃OD) δ 8.80 (dd, J=2.5, 0.7 Hz, 1H), 8.55 (td, J=1.9, 0.6 Hz, 1H), 8.31 (ddd, J=8.0, 2.0, 1.1 Hz, 1H), 8.22 (ddd, J=7.9, 1.8, 1.1 Hz, 1H), 7.89 (dd, J=9.7, 2.5 Hz, 1H), 7.76 (td, J=7.9, 0.5 Hz, 1H), 6.45 (dd, J=9.6, 0.7 Hz, 1H), 4.17 (d, J=2.6 Hz, 2H), 3.35 (t, J=7.2 Hz, 2H), 2.64 (t, J=2.6 Hz, 1H), 2.21 (t, J=7.3 Hz, 2H), 1.69-1.51 (m, 4H), 1.44 (s, 9H), 1.42-1.30 (m, 8H). ¹³C NMR (101 MHz, CD₃OD) δ 175.1, 167.2, 165.7, 161.4, 141.6, 138.2, 136.4, 135.2, 135.1, 134.0, 130.7, 129.8, 122.9, 116.4, 81.3, 80.4, 72.4, 41.1, 36.4, 30.4, 30.3, 30.3, 30.1, 30.1, 28.4, 28.0, 26.2. IR (neat, vₘₐₓ/cm⁻¹) 3298, 3073, 2928, 2856, 1726, 1692, 1638, 1534, 1460, 1368, 1300, 1252, 1187, 1153, 1096. HRMS (ESI): m/z=594.2245 [M+Na]⁺ (calc. for C₂₉H₃₇N₃NaO₇S m/z=594.2244)

### Step c) tert-butyl 9-(1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamido)nonanoate

To a vial containing N-(2-(2-azidoethoxy)ethyl)-7-nitrobenzo[c][1,2,5]oxadiazol-4-amine (7.0 mg, 23.7 μmol, 2.0 equiv) and tert-butyl 9-(6-oxo-1-((3-(prop-2-yn-1-ylcarbamoyl)phenyl)sulfonyl)-1,6-dihydropyridine-3-carboxamido)nonanoate (6.8 mg, 11.8 μmol, 1.0 equiv) was added [Cu(MeCN)₄]PF₆ (22.2 mg, 59.4 μmol, 5.0 equiv) in the glovebox. The mixture was dissolved in argon degassed MeOH (0.12 mL) and CH₂Cl₂ (0.12 mL) and the reaction mixture was heated to 40° C. and stirred for 16 h under argon. The reaction mixture was directly loaded on SiO₂ plate and the crude product was purified by preparative TLC (SiO₂, 2-6% MeOH in CH₂Cl₂) and triturated with hexane to afford the title product as an orange solid (7.5 mg, 73%).

¹H NMR (400 MHz, CD₃OD) δ 8.77 (dd, J=2.5, 0.7 Hz, 1H), 8.53 (t, J=1.8 Hz, 1H), 8.43 (d, J=8.9 Hz, 1H), 8.27 (ddd, J=8.0, 2.0, 1.1 Hz, 1H), 8.19 (ddd, J=7.9, 1.7, 1.1 Hz, 1H), 7.92 (s, 1H), 7.85 (dd, J=9.7, 2.5 Hz, 1H), 7.72 (td, J=7.9, 0.5 Hz, 1H), 6.40 (dd, J=9.7, 0.7 Hz, 1H), 6.31 (d, J=8.9 Hz, 1H), 4.64-4.49 (m, 4H), 3.94-3.86 (m, 2H), 3.79-3.71 (m, 2H), 3.69 (bs, 2H), 3.34 (d, J=7.2 Hz, 2H), 2.21 (t, J=7.3 Hz, 2H), 1.70-1.52 (m, 4H), 1.43 (s, 9H), 1.40-1.23 (m, 8H). ¹³C NMR (101 MHz, CD₃OD) δ 175.2, 167.5, 165.6, 161.4, 145.8, 141.5, 138.1, 136.5, 135.2, 135.0, 133.9, 130.6, 129.8, 125.3, 122.9, 116.4, 81.3, 70.3, 70.1, 51.4, 41.2, 36.4, 36.3, 30.3, 30.3, 30.1, 28.4, 28.0, 26.2. IR (neat, vₘₐₓ/cm⁻¹) 3300, 3075, 2925, 2854, 1727, 1691, 1643, 1583, 1532, 1456, 1368, 1301, 1257, 1189, 1148. HRMS (ESI): m/z=865.3298 [M+H]⁺ (calc. for 5 C₃₉H₄₉N₁₀O₁₁S m/z=865.3296)

### Step d) N-(9-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-9-oxononyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1, 6-dihydropyridine-3-carboxamide

To a solution of tert-butyl 9-(1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamido)nonanoate (3.0 mg, 3.4 μmol, 1.0 equiv) in dry CH₂Cl₂ (200 μL) was added TFA (100 μL) and the reaction mixture was allowed to stir for 30 min. at rt. Then, the solvents were removed in vacuoo, the residue was re-dissolved in CH₂Cl₂ and co-evaporated (×3). The residue was then dissolved in dry DMF (50 μL). TFA (0.5 M solution in dry DMF, 22.9 μL, 11.4 μmol, 3.3 equiv) and i-Pr₂NEt (0.5 M solution in dry DMF, 36.8 μL, 18.3 μmol, 5.3 equiv) were added at rt. and the resulting solution was cooled to 0° C. Then, HATU (0.1 M solution in DMF, 38.2 μL, 3.8 μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 38.2 μL, 3.8 μmol, 1.1 equiv) was added. The reaction mixture was then allowed to warm up to ambient temperature and stirred for further 45 min. The residue was taken up in CH₂Cl₂ (10 mL) and washed with sat. aq. NH₄Cl(5 mL), sat. aq. NaHCO₃ (5 mL) and brine (5 mL). The organic layer was dried over Na₂SO₄, filtered and concentrated in vacuoo. The crude product was purified by flash column chromatography (SiO₂, 4% MeOH in CH₂Cl₂) to afford the product (3.5 mg, 81%) as an orange solid.

¹H NMR (500 MHz, CD₂Cl₂) δ 8.59 (dd, J=2.5, 0.7 Hz, 1H), 8.46 (d, J=8.7 Hz, 1H), 8.40 (t, J=1.7 Hz, 1H), 8.25 (ddd, J=8.0, 1.9, 1.1 Hz, 1H), 8.11 (d, J=7.8 Hz, 1H), 7.72 (dd, J=9.6, 2.5 Hz, 1H), 7.70 (s, 1H), 7.63 (t, J=7.9 Hz, 1H), 7.43 (bs, 1H), 6.57 (t, J=5.9 Hz, 1H), 6.49 (s, 2H), 6.36 (dd, J=9.7, 0.6 Hz, 1H), 6.23 (d, J=8.7 Hz, 1H), 5.58 (dt, J=3.0, 1.5 Hz, 1H), 5.48 (t, J=5.7 Hz, 1H), 4.64 (d, J=5.6 Hz, 2H), 4.53 (t, J=4.9 Hz, 2H), 3.96-3.93 (m, 1H), 3.90 (t, J=5.0 Hz, 2H), 3.87-3.77 (m, 2H), 3.77-3.73 (m, 2H), 3.72 (s, 6H), 3.66 (bs, 2H), 3.40-3.33 (m, 2H), 3.22 (t, J=7.0 Hz, 2H), 2.21-2.13 (m, 4H), 2.11-1.98 (m, 3H), 1.67 (d, J=8.4 Hz, 1H), 1.63-1.50 (m, 6H), 1.36-1.21 (m, 12H), 1.27 (s, 3H), 1.26 (s, 6H), 1.15-1.06 (m, 2H), 0.94 (s, 3H). ¹³C NMR (126 MHz, CD₂Cl₂) δ 173.3, 165.5, 163.7, 160.0, 159.0, 150.0, 140.4, 139.2, 137.2, 137.0, 135.8, 134.2, 133.7, 133.4, 129.8, 128.7, 124.6, 124.0, 123.0, 118.0, 115.8, 103.3, 69.8, 69.1, 56.2, 52.0, 50.8, 48.0, 44.9, 44.8, 41.2, 40.7, 38.5, 38.0, 37.3, 36.1, 32.5, 30.4, 30.2, 29.6, 29.5, 29.4, 29.3, 28.1, 27.7, 27.2, 27.1, 26.6, 26.2, 25.1, 23.3, 21.3. IR (neat, vₘₐₓ/cm⁻¹) 3322, 2924, 2853, 2096, 1651, 1534, 1463, 1411, 1377, 1302, 1189, 1123. HRMS (ESI): m/z=645.2793 [M+Na₂]²⁺ (calc. for C₆₂H₈₀N₁₄Na₂O₁₂S m/z=645.2793)

### Example 8

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide

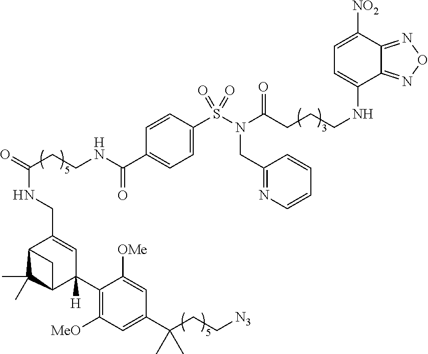

### Step a) tert-butyl 7-(4-(N-(pyridin-2-ylmethyl)sulfamoyl)benzamido)heptanoate

Solution of 4-chlorosulfonylbenzoyl chloride (855 mg, 3.56 mmol, 3.75 equiv, CAS RN 7516-60-1) in dry CH₂Cl₂ (30 mL) was cooled to 0° C. and Et₃N (1.04 mL, 5.96 mmol, 6.25 equiv) was added dropwise followed by tert-butyl 7-aminoheptanoate (240 g. 954 μmol, 1.00 equiv, CAS RN 105974-64-9). The solution was stirred at 0° C. for 1 h, concentrated in vacuoo and the crude roughly purified by flash column chromatography (SiO₂, 10% EtOAc in CHCl₃). The sulfonyl chloride was immediately used in the subsequent step.

Tert-butyl 7-(4-(chlorosulfonyl)benzamido)heptanoate (260 mg, 644 μmol, 1.0 equiv) was dissolved in dry dioxane (1.5 mL). Pyridin-2-ylmethanamine (73 μL, 708 μmol, 1.1 equiv) and i-Pr₂NEt (170 μL, 966 μmol, 1.5 equiv) were added and the solution was stirred at rt overnight. The mixture was diluted with EtOAc (50 mL) and aq. sat. NH₄Cl solution (50 5 mL). The layers were separated and the aqueous phase was extracted with EtOAc (2×50 mL). The combined organic phase was washed with brine (30 mL), dried with MgSO₄, filtered and concentrated in vacuoo. The crude product was purified by flash column chromatography (SiO₂, 70% EtOAc in hexanes) to yield the title compound as an amorphous solid (232 mg, 76%).

¹H NMR (500 MHz, CD₃Cl) δ 8.44 (ddd, J=4.9, 1.8, 1.0 Hz, 1H), 7.94-7.86 (m, 2H), 7.83-7.78 (m, 2H), 7.60 (td, J=7.7, 1.8 Hz, 1H), 7.19-7.12 (m, 2H), 6.27 (bs, 1H), 6.16 (bs, 1H), 4.26 (d, J=5.1 Hz, 2H), 3.44 (td, J=7.2, 5.8 Hz, 2H), 2.21 (t, J=7.4 Hz, 2H), 1.71-1.52 (m, 4H), 1.43 (s, 9H), 1.41-1.30 (m, 4H). ¹³C NMR (126 MHz, CD₃Cl) δ 173.34, 166.16, 154.37, 149.14, 142.28, 138.76, 137.04, 127.74, 127.56, 122.93, 122.05, 80.26, 47.36, 40.27, 35.53, 29.45, 28.71, 28.26, 26.67, 25.00. IR (neat, vₘₐₓ/cm⁻¹): 3289, 2931, 2859, 1726, 1645, 1597, 1543, 1439, 1393, 1367, 1334, 1152, 1094. HRMS (ESI): m/z=498.2034 [M+Na]⁺ (calc. for C₂₄H₃₃N₃NaO₅S m/z=498.2033)

### Step b) tert-butyl 7-(4-(N-(6-((7-nitrobenzo[c][1,2,5]oxadiazol-4-yl)amino)hexanoyl)-N-(pyridin-2-ylmethyl)sulfamoyl)benzamido)heptanoate

6-((7-nitrobenzo[c][1,2,5]oxadiazol-4-yl)amino)hexanoic acid (14.5 mg, 44.4 μmol, 1.25 equiv, CAS RN 88235-25-0), EDCI·HCl (14.0 mg, 73.0 μmol, 2.00 equiv), i-Pr₂NEt (18.6 μL, 106 μmol, 3.00 equiv) and DMAP (5.4 mg, 44.4 μmol, 1.25 equiv) were dissolved in dry DMF (200 μL) and the mixture was stirred for 5 min at rt. Tert-butyl 7-(4-(N-(pyridin-2-ylmethyl)sulfamoyl)benzamido)heptanoate (16.9 mg, 35.5 μmol, 1.00 equiv) was added in dry DMF (100 μL) and the reaction mixture was stirred at rt. overnight. The mixture was partitioned between CH₂Cl₂ (20 mL) and aq. sat. NH₄Cl solution (10 mL). The layers were separated and the aqueous phase was extracted with CH₂Cl₂ (2×20 mL). The combined organic layer was dried with MgSO₄, filtered and concentrated in vacuoo. The crude product was purified by flash column chromatography (SiO₂, 70% EtOAc in hexanes) to yield the title compound as an orange solid (6.1 mg, 23%).

¹H NMR (500 MHz, CD₂Cl₂) δ 8.49-8.44 (m, 2H), 7.93-7.87 (m, 2H), 7.84-7.80 (m, 2H), 7.73 (td, J=7.7, 1.8 Hz, 1H), 7.36 (d, J=7.8 Hz, 1H), 7.23 (ddd, J=7.6, 4.9, 1.1 Hz, 1H), 6.50 (bs, 1H), 6.29 (t, J=5.7 Hz, 1H), 6.16 (d, J=8.7 Hz, 1H), 5.19 (s, 2H), 3.50-3.37 (m, 4H), 2.61 (t, J=7.1 Hz, 2H), 2.19 (t, J=7.4 Hz, 2H), 1.75-1.65 (m, 2H), 1.64-1.52 (m, 6H), 1.42 (s, 9H), 1.40-1.26 (m, 6H). ¹³C NMR (126 MHz, CD₂Cl₂) δ 173.54, 173.34, 166.00, 156.01, 149.72, 144.86, 144.50, 142.08, 140.15, 137.36, 137.10, 129.16, 127.56, 123.21, 122.06, 99.07, 80.15, 51.10, 44.04, 40.52, 35.88, 35.75, 29.71, 29.01, 28.41, 28.23, 26.94, 26.35, 25.32, 23.99. IR (neat, vₘₐₓ/cm⁻¹): 3335, 2926, 2856, 1708, 1649, 1622, 1585, 1531, 1490, 1445, 1365, 1298, 1261, 1169, 1123, 1088. HRMS (ESI): m/z=774.2904 [M+Na]⁺ (calc. for C₃₆H₄₅N₇NaO₉S m/z=774.2892)

### Step c) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide

Tert-butyl 7-(4-(N-(6-((7-nitrobenzo[c][1,2,5]oxadiazol-4-yl)amino)hexanoyl)-N-(pyridin-2-ylmethyl)sulfamoyl)benzamido)heptanoate (6.4 mg, 8.4 μmol, 1.1 equiv) was dissolved in a mixture of CH₂Cl₂ (200 μL) and TFA (100 μL) and the solution was stirred at rt for 2h. Volatiles were removed in vacuoo and the residue was dissolved in dry DMF (0.1 mL) and cooled to 0° C. Subsequently, stock solutions of TFA (0.5 M in DMF, 69 μL, 34.6 μmol, 4.5 equiv), i-Pr₂NEt (0.5 M in DMF, 123 μL, 61.6 μmol, 8.0 equiv) and HATU (0.1 M in DMF, 115 μL, 11.5 μmol, 1.5 equiv) were added and the mixture was stirred at 0° C. for 15 min. ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (3.5 mg, 7.6 μmol, 1.0 equiv) was dissolved in dry CH₂Cl₂ (50 μL), added and the reaction mixture was allowed to warm up to rt and stirred for 45 min. The crude mixture was concentrated in vacuoo, purified by preparative TLC (SiO₂, 95% EtOAc in hexanes) and triturated with hexane to afford the title compound as an orange solid (6.2 mg, 71%).

¹H NMR 1H NMR (500 MHz, CD₂Cl₂) δ 8.52-8.48 (m, 1H), 8.47 (d, J=8.7 Hz, 1H), 7.93 (d, J=8.5 Hz, 2H), 7.90-7.80 (m, 3H), 7.47 (d, J=7.8 Hz, 1H), 7.34 (bs, 1H), 6.59 (bs, 1H), 6.48 (s, 2H), 6.16 (d, J=8.7 Hz, 1H), 5.58 (dt, J=2.9, 1.4 Hz, 1H), 5.45 (t, J=5.7 Hz, 1H), 5.26 (s, 2H), 3.98-3.92 (m, 1H), 3.88-3.75 (m, 2H), 3.72 (s, 6H), 3.48-3.37 (m, 4H), 3.21 (t, J=7.0 Hz, 2H), 2.64 (t, J=7.0 Hz, 2H), 2.22-2.12 (m, 3H), 2.07 (td, J=5.7, 1.4 Hz, 1H), 2.03-1.98 (m, 1H), 1.76-1.48 (m, 13H), 1.44-1.21 (m, 1OH), 1.27 (s, 3H), 1.26 (s, 6H), 1.16-1.05 (m, 2H), 0.94 (s, 3H). ¹³C NMR (126 MHz, CD₂Cl₂) δ 173.48, 172.89, 165.92, 158.91, 155.65, 149.92, 140.37, 139.09, 137.09, 129.05, 127.86, 123.91, 117.92, 103.22, 56.13, 51.93, 47.87, 44.85, 44.64, 44.09, 41.11, 40.40, 38.36, 37.92, 36.94, 36.02, 32.36, 30.25, 30.12, 29.58, 29.23, 29.14, 28.98, 28.44, 27.98, 27.02, 26.79, 26.46, 26.40, 25.98, 25.02, 24.08, 21.17. IR (neat, vₘₐₓ/cm⁻¹): 3315, 2925, 2855, 2095, 1707, 1647, 1622, 1573, 1531, 1448, 1410, 1353, 1297, 1261, 1239, 1169, 1120, 1088, 1032. HRMS (ESI): m/z=1154.5472 [M+H]⁺ (calc. for C₅₉H₇₇NNNaO₁₀S m/z=1154.5468)

### Example 9

### N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide

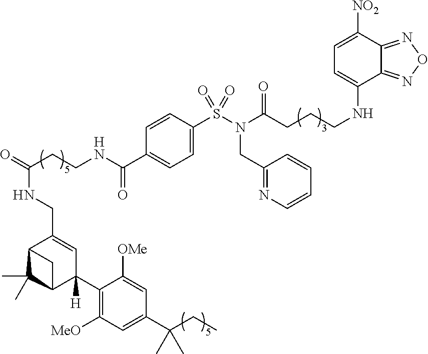

### Step c) N-(7-((((1S,4S,5S)-4-(2,6-dimethoxy-4-(2-methyloctan-2-yl)phenyl)-6, 6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide

Tert-butyl 7-(4-(N-(6-((7-nitrobenzo[c][1,2,5]oxadiazol-4-yl)amino)hexanoyl)-N-(pyridin-2-ylmethyl)sulfamoyl)benzamido)heptanoate (3.0 mg, 3.9 μmol, 1.0 equiv) was dissolved in a mixture of CH₂Cl₂ (200 μL) and TFA (100 μL) and the solution was stirred at rt for 2h. Volatiles were removed in vacuoo and the residue was dissolved in dry DMF (0.1 mL) and cooled to 0° C. Subsequently, stock solutions of TFA (0.5 M in DMF, 35.9 μL, 17.9 μmol, 4.5 equiv), i-Pr₂NEt (0.5 M in DMF, 60 μL, 29.9 μmol, 7.5 equiv) and HATU (0.1 M in DMF, 60 μL, 5.9 μmol, 1.5 equiv) were added and the mixture was stirred at 0° C. for 15 min. ((1S,4S,5S)-4-(2,6-dimethoxy-4-(2-methyloctan-2-yl)phenyl)-6,6-dimethylbicyclo[3.1.1]hept-2-en-2-yl)methanamine (1.7 mg, 4.1 μmol, 1.05 equiv) was dissolved in dry CH₂Cl₂ (50 μL), added and the reaction mixture was allowed to warm up to rt and stirred for 45 min. Additional i-Pr₂NEt (0.5 M in DMF, 10 μL, 5.0 μmol, 1.25 equiv) was added and the reaction mixture was stirred at rt for 1.5 h. The crude mixture was concentrated in vacuoo, purified by preparative TLC (SiO₂, 95% EtOAc in hexanes) and triturated with hexane to afford the title compound as an orange solid (4.2 mg, 96%).

¹H NMR 1H NMR (500 MHz, CD₂Cl₂) δ 8.50-8.42 (m, 2H), 7.93-7.88 (m, 2H), 7.87-7.80 (m, 2H), 7.72 (td, J=7.7, 1.8 Hz, 1H), 7.36 (dt, J=7.8, 1.0 Hz, 1H), 7.23 (ddd, J=7.6, 4.8, 1.1 Hz, 1H), 6.52 (s, 1H), 6.49 (s, 2H), 6.43 (t, J=5.8 Hz, 1H), 6.16 (d, J=8.7 Hz, 1H), 5.59 (dt, J=2.9, 1.4 Hz, 1H), 5.47-5.41 (m, 1H), 5.19 (s, 2H), 3.95 (t, J=2.3 Hz, 1H), 3.89-3.74 (m, 2H), 3.72 (s, 6H), 3.48-3.37 (m, 4H), 2.61 (t, J=7.0 Hz, 2H), 2.22-2.13 (m, 3H), 2.08 (td, J=5.7, 1.4 Hz, 1H), 2.05-1.97 (m, 1H), 1.74-1.54 (m, 13H), 1.45-1.19 (m, 1OH), 1.28 (s, 3H), 1.26 (s, 6H), 1.15-1.05 (m, 2H), 0.95 (s, 3H), 0.84 (t, J=4.4 Hz, 3H). ¹³C NMR (126 MHz, CD₂Cl₂) δ 173.51, 172.87, 165.96, 158.83, 156.04, 150.03, 149.74, 142.07, 140.14, 139.01, 137.34, 137.10, 129.12, 127.62, 123.90, 123.19, 122.05, 117.77, 103.17, 56.09, 51.11, 47.81, 44.78, 44.71, 44.61, 44.04, 41.07, 40.35, 38.34, 37.87, 36.92, 35.88, 32.18, 30.43, 30.10, 29.55, 29.16, 28.94, 28.42, 27.94, 26.75, 26.42, 26.36, 25.95, 25.08, 24.00, 23.07, 21.14, 14.25. IR (neat, vₘₐₓ/cm⁻¹): 3321, 2926, 2855, 1706, 1648, 1572, 1531, 1448, 1410, 1353, 1297, 1260, 1170, 1122, 1019. HRMS (ESI): m/z=1113.5443 [M+Na]⁺ (calc. for C₅₉H₇₈N₈NaO₁₀S m/z=1113.5454)

### Example 10

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(6-((7-nitrobenzo[c][1,2,5]oxadiazol-4-yl)amino)hexanoyl)sulfamoyl)benzamide

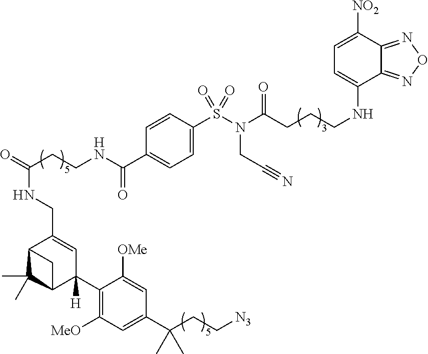

### Step a) tert-butyl 7-(4-sulfamoylbenzamido)heptanoate

To a solution of 4-sulfamoylbenzoic acid (550 mg, 2.73 mmol, 1.0 equiv, CAS RN 138-41-0) in dry DMF (11.0 mL) was added tert-butyl 7-aminoheptanoate (688 mg, 2.73 mmol, 1.0 equiv, CAS RN 105974-64-9), HOBt·H₂O (628 mg, 4.10 mmol, 1.5 equiv), EDCI·HCl (786 mg, 4.10 mmol, 1.5 equiv) and i-Pr₂NEt (1.43 mL, 8.20 mmol, 3.0 equiv). The solution was stirred at rt. for 16 h. The mixture was diluted with EtOAc (100 mL) and aq. sat. NaHCO₃ (50 mL). The layers were separated and the aqueous phase was extracted with EtOAc (2×100 mL). The combined organic phase was washed with brine (30 mL), dried with MgSO₄, filtered and concentrated in vacuoo. The crude product was purified by flash column chromatography (SiO₂, 4-6% MeOH in CH₂Cl₂) to yield the title compound as a white solid (700 mg, 67%).

¹H NMR (400 MHz, CD₃OD) δ 8.00-7.92 (m, 4H), 3.39 (t, J=7.2 Hz, 2H), 2.23 (t, J=7.3 Hz, 2H), 1.71-1.54 (m, 4H), 1.44 (s, 9H), 1.42-1.34 (m, 4H). ¹³C NMR (101 MHz, CD₃OD) δ 175.1, 168.7, 147.6, 139.2, 128.9, 127.3, 81.4, 41.1, 36.3, 30.2, 29.8, 28.3, 27.7, 26.1. IR (neat, vₘₐₓ/cm⁻¹): 3321, 2926, 2854, 1730, 1629, 1559, 1456, 1367, 1342, 1166, 1151, 1097. HRMS (ESI): m/z=407.1605 [M+Na]⁺ (calc. for C₁₈H₂₈N₂NaO₅S m/z=407.1611)

### Step b) tert-butyl 7-(4-(N-(6-((7-nitrobenzo[c][1,2,5]oxadiazol-4-yl)amino)hexanoyl)sulfamoyl)benzamido)heptanoate

6-((7-nitrobenzo[c][1,2,5]oxadiazol-4-yl)amino)hexanoic acid (112 mg, 343 μmol, 1.1 equiv, CAS RN 88235-25-0), EDCI·HCl (90.0 mg, 468 μmol, 1.5 equiv), i-Pr₂NEt (272 μL, 1.56 mmol, 5.0 equiv) and DMAP (11.4 mg, 93.6 μmol, 0.3 equiv) were dissolved in dry DMF (1.2 mL) and the mixture was stirred for 5 min at rt. Tert-butyl 7-(4-sulfamoylbenzamido)heptanoate (120 mg, 312 μmol, 1.0 equiv) was added and the reaction mixture was stirred at rt overnight. The mixture was concentrated in vacuoo. The crude product was purified by flash column chromatography (1% AcOH, 1-4% MeOH in CH₂Cl₂) to yield the title compound as an orange amorphous solid which was recrystallised in CHCl₃ (59 mg, 29%). The unreacted sulfonamide starting material was also recovered (62 mg).

¹H NMR (400 MHz, CD₃OD) δ 8.60 (t, J=5.7 Hz, 1H), 8.42 (d, J=8.8 Hz, 1H), 8.09-8.02 (m, 2H), 7.98-7.92 (m, 2H), 6.23 (d, J=8.9 Hz, 1H), 3.49-3.33 (m, 4H), 2.27 (t, J=7.2 Hz, 2H), 2.20 (t, J=7.4 Hz, 2H), 1.75-1.52 (m, 8H), 1.42 (s, 9H), 1.39-1.30 (m, 6H). ¹³C NMR (101 MHz, CD₃OD) δ 175.05, 173.63, 168.34, 146.50, 145.74, 145.43, 143.20, 140.67, 138.49, 129.34, 128.81, 122.86, 99.61, 81.34, 44.48, 41.13, 36.69, 36.30, 30.17, 29.77, 28.34, 27.71, 27.19, 26.07, 25.09. IR (neat, vₘₐₓ/cm⁻¹): 3288, 2932, 2859, 1721, 1641, 1622, 1584, 1531, 1495, 1447, 1366, 1297, 1170, 1151, 1088. HRMS (ESI): m/z=683.2466 [M+Na]⁺ (calc. for C₃₀H₄₀N₆NaO₉S m/z=683.2470)

### Step c) tert-butyl 7-(4-(N-(cyanomethyl)-N-(6-((7-nitrobenzo[c][1,2,5]oxadiazol-4-yl)amino)hexanoyl)sulfamoyl)benzamido)heptanoate

To a stirred solution of tert-butyl 7-(4-(N-(6-((7-nitrobenzo[c][1,2,5]oxadiazol-4-yl)amino)hexanoyl)sulfamoyl)benzamido)heptanoate (7.0 mg, 10.6 μmol, 1.0 equiv) in dry DMF (0.1 mL) was added iodoacetonitrile (8 μL, 106 μmol, 10 equiv) and i-Pr₂NEt (0.5 M in DMF, 106 μL, 53.0 μmol, 5.0 equiv). The mixture was stirred overnight at rt. The mixture was concentrated in vacuoo and the crude product was purified by preparative TLC (SiO₂, 70% EtOAc in hexanes) and triturated with hexane to yield the title compound as an orange solid (7.3 mg, 98%).

¹H NMR (500 MHz, CD₂Cl₂) δ 8.48 (d, J=8.6 Hz, 1H), 8.06-8.01 (m, 2H), 8.01-7.96 (m, 2H), 6.54 (bs, 1H), 6.35 (bs, 1H), 6.19 (d, J=8.7 Hz, 1H), 4.76 (s, 2H), 3.51-3.39 (m, 4H), 2.70 (t, J=7.1 Hz, 2H), 2.19 (t, J=7.4 Hz, 2H), 1.80-1.52 (m, 8H), 1.42 (s, 9H), 1.40-1.27 (m, 6H). ¹³C NMR (126 MHz, CD₂Cl₂) δ 173.36, 172.08, 165.64, 144.89, 144.50, 141.44, 140.69, 137.09, 128.78, 128.39, 115.16, 99.11, 80.18, 44.03, 40.62, 36.24, 35.73, 33.74, 29.64, 28.97, 28.50, 28.22, 26.92, 26.43, 25.29, 24.27. IR (neat, vₘₐₓ/cm⁻¹): 3338, 2926, 2856, 1716, 1653, 1621,1585,1532,1491,1447,1401,1367,1298,1169,1126, 1089. HRMS (ESI): m/z=722.2574 [M+Na]⁺ (calc. for C₃₂H₄₁N₇NaO₉S m/z=722.2579)

### Step d) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(6-((7-nitrobenzo[c][1,2,5]oxadiazol-4-yl)amino)hexanoyl)sulfamoyl)benzamide

tert-butyl 7-(4-(N-(cyanomethyl)-N-(6-((7-nitrobenzo[c][1,2,5]oxadiazol-4-yl)amino)hexanoyl)sulfamoyl)benzamido)heptanoate (3.6 mg, 5.1 μmol, 1.0 equiv) was dissolved in a mixture of CH₂Cl₂ (200 μL) and TFA (100 μL) and the solution was stirred at rt for 2 h. Volatiles were removed in vacuoo and the residue was dissolved in dry DMF (0.1 mL) and cooled to 0° C. Subsequently, stock solutions of TFA (0.5 M in DMF, 46 μL, 23.2 μmol, 4.5 equiv), i-Pr₂NEt (0.5 M in DMF, 82 μL, 41.2 μmol, 8.0 equiv) and HATU (0.1 M in DMF, 77 μL, 77.2 μmol, 1.5 equiv) were added and the mixture was stirred at 0° C. for 15 min. ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (2.4 mg, 5.1 μmol, 1.0 equiv) was dissolved in dry CH₂Cl₂ (50 μL), added and the reaction mixture was allowed to warm up to rt and stirred for 45 min. The crude mixture was concentrated in vacuoo, purified by preparative TLC (SiO₂, 95% EtOAc in hexanes) and triturated with hexane to afford the title compound as an orange solid (3.2 mg, 58%).

¹H NMR (500 MHz, CD₂Cl₂) δ 8.48 (d, J=8.7 Hz, 1H), 8.06-7.99 (m, 4H), 6.61 (bs, 1H), 6.56 (bt, J=5.7 Hz, 1H), 6.49 (s, 2H), 6.18 (d, J=8.7 Hz, 1H), 5.58 (dt, J=2.8, 1.5 Hz, 1H), 5.46 (bt, J=5.7 Hz, 1H), 4.76 (s, 2H), 3.98-3.92 (m, 1H), 3.89-3.74 (m, 2H), 3.72 (s, 6H), 3.53-3.38 (m, 4H), 3.22 (t, J=7.0 Hz, 2H), 2.70 (t, J=7.1 Hz, 2H), 2.23-2.12 (m, 3H), 2.10-2.04 (m, 1H), 2.04-1.95 (m, 1H), 1.76-1.47 (m, 13H), 1.43-1.29 (m, 1OH), 1.27 (s, 3H), 1.26 (s, 6H), 1.16-1.06 (m, 2H), 0.94 (s, 3H). ¹³C NMR (126 MHz, CD₂Cl₂) δ 173.03, 172.08, 165.64, 158.84, 149.90, 141.40, 140.67, 138.96, 137.11, 128.86, 128.34, 123.94, 117.81, 115.17, 103.16, 56.11, 51.90, 47.80, 44.79, 44.64, 44.61, 41.07, 40.36, 38.33, 37.87, 36.84, 36.23, 33.72, 30.22, 30.09, 29.35, 29.20, 29.11, 28.75, 28.48, 27.94, 26.99, 26.58, 26.41, 25.83, 24.99, 24.27, 21.13. IR (neat, vₘₐₓ/cm⁻¹): 3363, 2925, 2855, 2096, 1715, 1653, 1583, 1532, 1456, 1410, 1365, 1297, 1261, 1170, 1122, 1031. HRMS (ESI): m/z=1080.5312 [M+H]⁺ (calc. for C₅₅H₇₄N₁₁O₁₀S m/z=1080.5335)

### Example 11

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamide

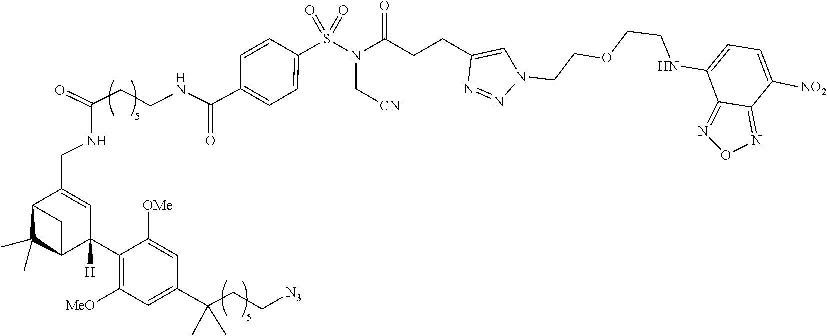

### Step g) tert-butyl 7-(4-(N-(pent-4-ynoyl)sulfamoyl)benzamido)heptanoate

Pent-4-ynoic acid (60.3 mg, 614 μmol, 1.05 equiv, CAS RN 6089-09-4), EDCI·HCl (168 mg, 878 μmol, 1.5 equiv), i-Pr₂NEt (510 μL, 2.93 mmol, 5.0 equiv) and DMAP (21.6 mg, 176 μmol, 0.30 equiv) were dissolved in dry DMF (1.3 mL) and the mixture was stirred for 15 min at rt. Subsequently, tert-butyl 7-(4-sulfamoylbenzamido)heptanoate (225 mg, 585 μmol, 1.0 equiv) was added in dry DMF (1.0 mL) and the reaction mixture was stirred at rt overnight. The mixture was partitioned between CH₂Cl₂ (60 mL) and aq. sat. NH₄Cl solution (20 mL). The layers were separated and the aqueous phase was extracted with CH₂Cl₂ (2×60 mL). The combined organic layer was dried with MgSO₄, filtered and concentrated in vacuoo. The crude product was purified by flash column chromatography (SiO₂, 0.5% AcOH, 0-4% MeOH in CH₂Cl₂) to yield the title compound as a white solid (190 mg, 70%), unreacted sulfonamide starting material was also recovered (52 mg).

¹H NMR (400 MHz, CD₃OD) δ 8.68 (bt, J=5.8 Hz, 1H), 8.12-8.05 (m, 2H), 8.02-7.93 (m, 2H), 3.47-3.34 (m, 2H), 2.48-2.43 (m, 2H), 2.39-2.33 (m, 2H), 2.23 (t, J=7.3 Hz, 2H), 2.19 (t, J=2.6 Hz, 1H), 1.71-1.53 (m, 4H), 1.44 (s, 9H), 1.42-1.33 (m, 4H). ¹³C NMR (126 MHz, CD₃OD) δ 175.07, 171.88, 168.53, 143.24, 140.72, 129.40, 128.82, 82.81, 81.36, 70.46, 41.12, 36.31, 35.99, 30.20, 29.80, 28.34, 27.72, 26.10, 14.36. IR (neat, vₘₐₓ/cm⁻¹): 3254, 2928, 2856, 2510, 1712, 1635, 1570, 1480, 1464, 1434, 1415, 1351, 1172. HRMS (ESI): m/z=487.1862 [M+Na]⁺ (calc. for C₂₃H₃₂N₂NaO₆S m/z=487.1873)

### Step h) tert-butyl 7-(4-(N-(cyanomethyl)-N-(pent-4-ynoyl)sulfamoyl)benzamido)heptanoate

To a solution of tert-butyl 7-(4-(N-(pent-4-ynoyl)sulfamoyl)benzamido)heptanoate (75.0 mg, 161 μmol, 1.0 equiv) in dry DMF (0.25 ml) was added i-Pr₂NEt (141 μL, 807 μmol, 5.0 equiv) and iodoacetonitrile (117 μL, 1.61 mmol, 10 equiv). The clear reaction mixture turned brown after 10 min. and was allowed to stir for further 16 h at rt. Then, the reaction mixture was concentrated in vacuoo, re-dissolved in CH₂Cl₂ and adsorbed on silica gel. Purification by flash column chromatography (SiO₂, 30% EtOAc in hexanes) afforded the product (63.5 mg, 78%) as a yellowish wax.

¹H NMR (500 MHz, CD₂Cl₂) 6=8.06-8.02 (m, 2H), 8.01-7.97 (m, 2H), 6.34 (s, 1H), 4.74 (s, 2H), 3.43 (td, J=7.2, 5.8 Hz, 2H), 2.97 (t, J=7.1 Hz, 2H), 2.49 (td, J=7.1, 2.7 Hz, 2H), 2.19 (t, J=7.4 Hz, 2H), 1.98 (t, J=2.7 Hz, 1H), 1.69-1.50 (m, 4H), 1.42 (s, 9H), 1.41-1.31 (m, 4H). ¹³C NMR (126 MHz, CD₂Cl₂) δ 173.5, 170.8, 165.6, 141.6, 140.5, 129.0, 128.5, 115.1, 82.4, 80.3, 69.7, 40.7, 36.1, 35.9, 33.9, 29.8, 29.1, 28.4, 27.1, 25.5, 14.4. IR (neat, vₘₐₓ/cm⁻¹) 3300, 2934, 2861, 1718, 1649, 1542, 1487, 1367, 1293, 1169, 1154, 1088, 1070, 1001. HRMS (ESI): m/z=526.1978 [M+Na]⁺ (calc. for C₂₅H₃₃N₃NaO₆S m/z=526.1982)

### Step j) tert-butyl 7-(4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamido)heptanoate

To a heart-shaped flask loaded with tert-butyl 7-(4-(N-(cyanomethyl)-N-(pent-4-ynoyl)sulfamoyl)benzamido)heptanoate (5.7 mg, 11.3 μmol, 1.0 equiv), N-(2-(2-azidoethoxy)ethyl)-7-nitrobenzo[c][1,2,5]oxadiazol-4-amine (5.0 mg, 16.9 μmol, 1.5 equiv, CAS RN 2449214-44-0) and Cu[MeCN]₄PF₆ (21.1 mg, 56.6 μmol, 5.0 equiv) was added dry CH₂Cl₂ (0.3 mL, previously degassed with argon) and 10 μL of AcOH. The flask was then flushed with argon, stoppered with a glass stopper and the orange solution was allowed to stir for 16 h. The reaction mixture was then directly loaded onto a silica plate and purified by preparative TLC (SiO₂, 8% MeOH in CH₂Cl₂) to afford the product (3.3 mg, 37%) as an orange solid.

¹H NMR (500 MHz, CD₂Cl₂) 6=8.48 (d, J=8.4 Hz, 1H), 8.05-7.85 (m, 4H), 7.34 (s, 1H), 6.73 (s, 1H), 6.49 (s, 1H), 6.23 (d, J=8.6 Hz, 1H), 4.74 (s, 2H), 4.53-4.41 (m, 2H), 3.88 (s, 2H), 3.74 (s, 2H), 3.65 (d, J=11.5 Hz, 2H), 3.43 (t, J=6.4 Hz, 2H), 3.17-3.08 (m, 2H), 3.00-2.92 (m, 2H), 2.19 (t, J=7.4 Hz, 2H), 1.67-1.52 (m, 4H), 1.42 (s, 9H), 1.38 (s, 4H). ¹³C NMR (126 MHz, CD₂Cl₂) 6=173.50, 172.16, 171.97, 165.94, 145.06, 141.59, 140.56, 137.17, 128.89, 128.49, 123.54, 122.95, 115.33, 98.84, 80.30, 69.94, 68.93, 50.60, 44.04, 40.64, 36.02, 35.92, 34.05, 29.84, 29.19, 28.39, 27.14, 25.48, 20.99. IR (neat, vₘₐₓ/cm⁻¹) 3325, 3080, 2932,2863, 1718, 1648, 1622, 1583, 1532, 1493, 1443, 1399, 1366, 1301, 1191, 1168, 1152, 1037. HRMS (ESI): m/z=819.2860 [M+Na]⁺ (calc. for C₃₅H₄₄N₁₀NaO₁₀S m/z=819.2855)

### Step k) N-(3-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-3-oxoheptyl)-4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamide

To a solution of tert-butyl 7-(4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamido)heptanoate (3.3 mg, 4.1 μmol, 1.0 equiv) in dry CH₂Cl₂ (200 μL) was added TFA (100 μL) and the resulting yellow reaction mixture was allowed to stir for 1 h. Then, the solvents were evaporated in vacuoo, the residue was re-dissolved in CH₂Cl₂ and co-evaporated (×3). The residue was then dissolved in dry DMF (0.1 mL). TFA (0.5 M solution in dry DMF, 27.3 μL, 13.7 μmol, 3.3 equiv) and i-Pr₂NEt (0.5 M solution in dry DMF, 43.9 μL, 30.0 μmol, 5.3 equiv) were added and the resulting light yellow solution was cooled to 0° C. Then, HATU (0.1 M solution in DMF, 45.6μ, 4.5. μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 45.6 μL, 4.6 μmol, 1.1 equiv) was added. The reaction mixture was then allowed to warm to ambient temperature and stirred for further 30 min. The reaction mixture was then concentrated in vacuoo, the residue taken up in CH₂Cl₂ and directly loaded onto a silica plate. Purification by preparative TLC (SiO₂, 7% MeOH in CH₂Cl₂) afforded the product (1.7 mg, 35%) as an orange solid. In absence of residual TFA the compound readily undergoes methanolysis.

¹H NMR (500 MHz, CD₂Cl₂) δ=8.46 (d, J=8.5 Hz, 1H), 8.04-7.87 (m, 4H), 7.71 (s, 1H), 6.83 (s, 1H), 6.49 (s, 2H), 6.23 (d, J=8.7 Hz, 1H), 5.91 (s, 1H), 5.62 (s, 1H), 4.73 (bs, 2H), 4.58 (bs, 2H), 3.96 (bs, 1H), 3.92 (bs, 2H), 3.85 (d, J=14.2 Hz, 2H), 3.79 (s, 2H), 3.72 (s, 6H), 3.70-3.66 (m, 2H), 3.43 (d, J=6.4 Hz, 2H), 3.22 (t, J=6.9 Hz, 2H), 3.14 (td, J=7.5, 4.0 Hz, 2H), 3.06 (s, 2H), 2.27-2.22 (m, 2H), 2.20-2.15 (m, 1H), 2.10-2.05 (m, 1H), 2.02 (t, J=4.6 Hz, 1H), 1.68 (d, J=8.4 Hz, 1H), 1.61-1.52 (m, 9H), 1.44-1.36 (m, 4H), 1.31-1.20 (m, 4H), 1.28 (s, 3H), 1.26 (s, 6H), 1.14-1.07 (m, 2H), 0.94 (s, 3H). ¹³C NMR (126 MHz, CD₂Cl₂) δ=178.48 (HMBC), 158.98, 147.61, 120.83, 120.62, 116.18, 112.43, 103.29, 102.47, 83.82, 56.26, 55.93, 52.06, 44.96, 44.76, 41.27, 40.60, 38.50, 38.05, 30.38, 29.88, 29.27, 27.15, 26.56, 25.14, 21.31. IR (neat, vₘₐₓ/cm⁻¹) 3325, 2929, 2864, 2097, 1717, 1635, 1574, 1541, 1449, 1410, 1364, 1302, 1172, 1122, 1030. HRMS (ESI): m/z=1199.5422 [M+Na]⁺ (calc. for C₅₈H₇₆N₁₄NaO₁₁S m/z=1199.5431)

### Example 12

### 1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

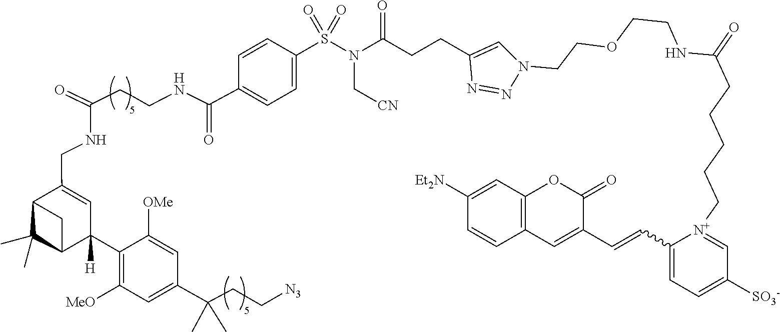

### Step j) 1-(6-((2-(2-(4-(3-((4-((7-(tert-butoxy)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

To a heart-shaped flask loaded with tert-butyl 7-(4-(N-(cyanomethyl)-N-(pent-4-ynoyl)sulfamoyl)benzamido)heptanoate (2.4 mg, 4.7 μmol, 1.5 equiv) and 1-(6-((2-(2-azidoethoxy)ethyl)amino)-6-oxohexyl)-6-(2-(7-(diethylamino)-2-oxo-2H-chromen-3-yl)vinyl)pyridin-1-ium-3-sulfonate (2.0 mg, 3.1 μmol, 1.0 equiv) was added [Cu(MeCN)₄PF₆](5.9 mg, 16.0 μmol, 5.0 equiv) in a glovebox. The solids were dissolved in dry, argon degassed CH₂Cl₂ (300 μL) and AcOH (10 μL). The flask was then flushed with argon, stoppered and the plum coloured solution was allowed to stir for 16 h at rt. The reaction mixture was then directly loaded onto a silica plate and purified by preparative TLC (SiO₂, 15% MeOH in CH₂Cl₂) to afford the product (3.4 mg, 94%) as a red solid.

¹H NMR (500 MHz, CD₃OD, E Z signal sets reported as full signals) δ=9.17 (dd, J=1.8, 0.6 Hz, 1H, Z), 9.02 (dd, J=1.9, 0.5 Hz, 1H, E), 8.58 (dd, J=8.7, 1.9 Hz, 1H, E), 8.51 (dd, J=8.3, 1.8 Hz, 1H, Z), 8.44 (d, J=8.6 Hz, 1H, E), 8.15 (s, 1H, E), 8.11 (d, J=15.3 Hz, 1H, E), 8.07 (d, J=8.8 Hz, 2H), 8.02-7.98 (m, 2H), 7.94 (s, 1H, Z), 7.89 (d, J=8.4 Hz, 1H, Z), 7.81 (d, J=15.5 Hz, 1H, E), 7.65 (s, 1H), 7.51 (d, J=9.1 Hz, 1H, E), 7.40 (d, J=9.1 Hz, 1H, Z), 7.10 (dd, J=12.3, 0.9 Hz, 1H, Z), 6.83 (dd, J=9.0, 2.5 Hz, 1H, E), 6.79 (d, J=12.3 Hz, 1H, Z), 6.75 (dd, J=9.0, 2.5 Hz, 1H, Z), 6.57 (dd, J=2.4, 0.6 Hz, 1H, E), 6.47 (d, J=2.4 Hz, 1H, Z), 4.74-4.63 (m, 2H), 4.48 (dd, J=5.5, 4.4 Hz, 2H), 3.79 (dd, J=5.6, 4.5 Hz, 2H), 3.58-3.52 (m, 4H), 3.51-3.47 (m, 4H), 3.42-3.35 (m, 2H), 3.35-3.30 (m, 2H), 3.16-3.10 (m, 2H), 2.99-2.92 (m, 2H), 2.28-2.18 (m, 4H), 2.11-1.97 (m, 2H), 1.74-1.67 (m, 2H), 1.65-1.57 (m, 4H), 1.54-1.46 (m, 2H), 1.43 (s, 9H), 1.41-1.36 (m, 4H), 1.25 (t, J=7.1 Hz, 6H, E), 1.22-1.18 (m, 6H, Z). ¹³C NMR (126 MHz, CD₃OD) δ 175.8, 175.0, 172.8, 162.1, 158.4, 155.4, 154.5, 153.8, 149.3, 146.9, 143.6, 142.8, 142.0, 141.6, 132.3, 129.6, 129.2, 128.4, 126.1, 124.4, 116.8, 116.7, 114.6, 111.7, 110.4, 97.6, 81.4, 70.5, 70.0, 51.3, 46.1, 40.2, 36.7, 36.3, 34.6, 30.2, 29.8, 28.4, 27.8, 26.8, 26.1, 21.4, 12.8. IR (neat, vₘₐₓ/cm⁻¹) 2924, 2854, 1710, 1620, 1579, 1556, 1504, 1459, 1423, 1357, 1278, 1198, 1171, 1136, 1053. HRMS (ESI): m/z=1152.4512 [M+Na]⁺ (calc. for C₅₅H₇₁N₉NaO₁₃S₂ m/z=1152.4505)

### Step k) 1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate

To a solution of 1-(6-((2-(2-(4-(3-((4-((7-(tert-butoxy)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate (2.5 mg, 2.2 μmol, 1.0 equiv) in dry CH₂Cl₂ (200 μL) was added TFA (100 μL) and the resulting plum coloured reaction mixture was allowed to stir for 30 min. Then, the solvents were removed in vacuoo, the residue was redissolved in CH₂Cl₂ and co-evaporated (×3). The residue was then dissolved in dry DMF (50 μL). TFA (0.5 M solution in dry DMF, 14.6 μL, 7.2 μmol, 3.3 equiv) and i-Pr₂NEt (0.5 M solution in dry DMF, 23.4 μL, 11.7 μmol, 5.3 equiv) were added and the resulting colourless solution was cooled to 0° C. Then, HATU (0.1 M solution in DMF, 24.3 μL, 2.4 μmol, 1.1 equiv) was added and the reaction was stirred at 0° C. for 15 min. Then amine ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.1 M solution in dry CH₂Cl₂, 24.3 μL, 2.4 μmol, 1.1 equiv) was added. The reaction mixture was allowed to warm up to ambient temperature and stirred for further 30 min. The reaction mixture was then concentrated in vacuoo, the residue taken up in CH₂Cl₂ and directly loaded onto a silica plate. Purification by preparative TLC (SiO₂, 7% MeOH in CH₂Cl₂) afforded the product (2.2 mg, 66%) as a red solid.

HRMS (ESI): m/z=755.8670 [M+2H]²⁺ (calc. for C₇₈H₁₀₅N₁₃O₁₄S₂ m/z=755.8667)

### Example 13

### 3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide

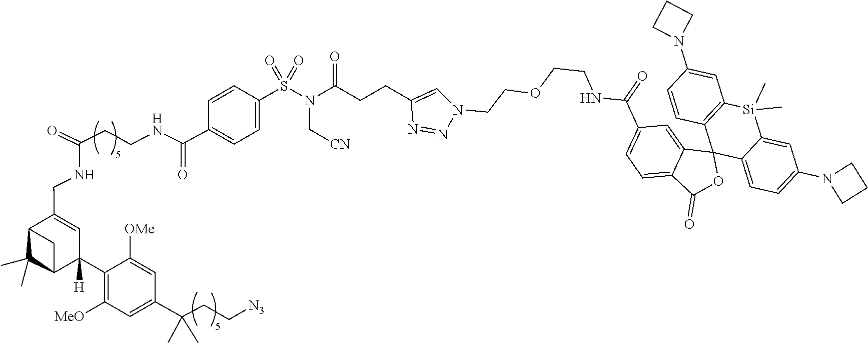

### Step j) tert-butyl 7-(4-(N-(cyanomethyl)-N-(3-(1-(2-(2-(3, 7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)propanoyl)sulfamoyl)benzamido)heptanoate

To a heart-shaped flask loaded with tert-butyl 7-(4-(N-(cyanomethyl)-N-(pent-4-ynoyl)sulfamoyl)benzamido)heptanoate (3.1 mg, 6.2 μmol, 1.5 equiv), 3,7-di(azetidin-1-yl)-N-(2-(2-azidoethoxy)ethyl)-5,5-dimethyl-3′-oxo-3′H,5H-spiro[dibenzo[b,e]siline-10,1′-isobenzofuran]-6′-carboxamide (2.5 mg, 4.1 μmol, 1.0 equiv) and Cu[MeCN]₄PF₆ (7.7 mg, 20.5 μmol, 5.0 equiv) was added dry CH₂Cl₂ (0.3 mL, previously degassed with argon) and 10 μL of AcOH. The flask was then flushed with argon, stoppered with a glass stopper and the deep blue solution was allowed to stir for 16 h. The reaction mixture was then directly loaded onto a silica plate and purified by preparative TLC (SiO₂, 3% MeOH in CH₂Cl₂) to afford the product (3.1 mg, 68%) as a blue solid.

¹H NMR (500 MHz, CD₂Cl₂) δ=8.09-7.85 (m, 6H), 7.74 (d, J=4.9 Hz, 1H), 7.29 (s, 1H), 6.83-6.68 (m, 4H), 6.37-6.28 (m, 2H), 4.70 (s, 2H), 4.43-4.37 (m, 2H), 3.96 (s, 8H), 3.81-3.76 (m, 2H), 3.59-3.50 (m, 4H), 3.33 (td, J=6.6, 2.2 Hz, 2H), 3.05 (t, J=13.4 Hz, 2H), 2.87-2.81 (m, 2H), 2.44-2.35 (m, 4H), 2.17 (t, J=10.3 Hz, 2H), 1.68-1.48 (m, 4H), 1.41 (s, 9H), 1.36-1.30 (m, 4H), 0.61 (d, J=1.7 Hz, 3H), 0.56 (d, J=2.4 Hz, 3H). ¹³C NMR (126 MHz, CD₂Cl₂) δ=171.90, 145.87, 140.47, 129.06, 128.31, 122.83 (HMBC), 122.37 (HMBC), 86.61, 80.50, 80.27, 69.88, 69.51, 69.46, 50.51, 43.32, 40.73, 40.44, 40.31, 36.08, 35.93, 33.93, 30.26, 29.79, 29.21, 28.40, 27.17, 25.50, 20.96, 17.29 (HSQC), 14.44 (HSQC), 9.07, -1.07. IR (neat, vₘₐₓ/cm⁻¹) 3344, 2926, 2855, 1755, 1722, 1654, 1594, 1546, 1480, 1365, 1291, 1235, 1159, 1091. HRMS (ESI): m/z=1134.4562 [M+Na]⁺ (calc. for C₅₈H₆₉N₉NaO₁₀SSi m/z=1134.4550)

### Step k) 3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide

To a solution of tert-butyl 7-(4-(N-(cyanomethyl)-N-(3-(1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)propanoyl)sulfamoyl)benzamido)heptanoate (3.1 mg, 2.8 μmol, 1.0 equiv) in dry CH₂Cl₂ (200 μL) was added TFA (100 μL) and the resulting deep blue reaction mixture was allowed to stir for 1 h. Then, the solvents were concentrated in vacuoo, the residue was re-dissolved in CH₂Cl₂ and co-evaporated (×3). The residue was then dissolved in dry DMF (0.1 mL). TFA (0.5 M solution in dry DMF, 18.4 μL, 9.2 μmol, 3.3 equiv) and i-Pr₂NEt (0.5 M solution in dry DMF, 35.1 μL, 17.6 μmol, 6.3 equiv) were added and the resulting colorless solution was cooled to 0° C. Then, HATU (0.1 M solution in DMF, 30.7 μL, 3.1. μmol, 1.1 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (0.05 M solution in dry CH₂Cl₂, 61.3 μL, 3.1 μmol, 1.1 equiv) was added. Monitoring of the reaction by LCMS showed incomplete conversion and additional i-Pr₂NEt (0.5 M solution in dry DMF, 11.1 μL, 5.6 μmol, 2.0 equiv) was added until full conversion was achieved. The reaction mixture was then allowed to warm to ambient temperature and stirred for further 30 min. The reaction mixture was then concentrated in vacuoo, the residue taken up in CH₂Cl₂ and directly loaded onto a silica plate. Purification by preparative TLC (SiO₂, 0.5% AcOH, 7% MeOH in CH₂Cl₂) afforded the product (2.1 mg, 38%). HRMS (ESI): m/z=1492.7302 [M+H]⁺ (calc. for C₈₁H₁₀₂N₁₃O₁₁SSi m/z=1492.7306)

### Example 14

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide

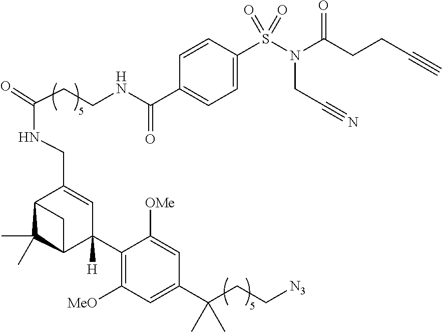

### Step i) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide

tert-butyl 7-(4-(N-(cyanomethyl)-N-(pent-4-ynoyl)sulfamoyl)benzamido)heptanoate (2.4 mg, 4.8 μmol, 1.1 equiv) was dissolved in a mixture of CH₂Cl₂ (200 μL) and TFA (100 μL) and the solution was stirred at rt for 1 h. Volatiles were removed in vacuoo and the residue was dissolved in dry DMF (50 μL) and cooled to 0° C. Subsequently, stock solutions of TFA (0.5 M in DMF, 29 μL, 14.5 μmol, 3.3 equiv), i-Pr₂NEt (0.5 M in DMF, 47 μL, 23.3 μmol, 5.3 equiv) and HATU (0.1 M in DMF, 48 μL, 4.8 μmol, 1.1 equiv) were added and the mixture was stirred at 0° C. for 15 min. ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (2.0 mg, 4.3 μmol, 1.0 equiv) was dissolved in dry CH₂Cl₂ (50 μL), added and the reaction mixture was allowed to warm up to rt. and stirred for 10 min. The crude mixture was concentrated in vacuoo, purified by preparative TLC (SiO₂, 85% EtOAc in hexanes) to afford the title compound as a colourless solid (2.0 mg, 51%).

¹H NMR 1H NMR (500 MHz, CD₂Cl₂) δ 8.07-8.00 (m, 4H), 6.49 (s, 2H), 6.47 (d, J=6.0 Hz, 1H), 5.61-5.56 (m, 1H), 5.42 (t, J=5.8 Hz, 1H), 4.74 (s, 2H), 3.98-3.93 (m, 1H), 3.88-3.74 (m, 2H), 3.72 (s, 6H), 3.48-3.39 (m, 2H), 3.22 (t, J=7.0 Hz, 2H), 2.97 (dd, J=7.4, 6.8 Hz, 2H), 2.49 (ddd, J=7.5, 6.8, 2.6 Hz, 2H), 2.22-2.13 (m, 3H), 2.08 (td, J=5.7, 1.4 Hz, 1H), 2.05-1.98 (m, 1H), 1.98 (t, J=2.7 Hz, 1H), 1.72-1.48 (m, 7H), 1.47-1.36 (m, 4H), 1.35-1.22 (m, 6H), 1.28 (s, 3H), 1.26 (s, 6H), 1.17-1.06 (m, 2H), 0.95 (s, 3H). ¹³C NMR (126 MHz, CD₂Cl₂) δ 173.04 (HMBC), 170.81 (HMBC), 165.51, 159.01, 150.04, 140.50, 139.17, 129.07, 128.51, 124.03, 117.98, 115.08, 103.30, 82.37, 69.71, 56.26, 52.06, 47.97, 44.95, 44.77, 41.23, 40.53, 38.49, 38.04, 37.02, 36.07, 33.93, 32.50, 30.39, 30.26, 29.62, 29.36, 29.28, 28.98, 28.11, 27.16, 26.81, 26.58, 26.06, 25.15, 21.30. IR (neat, vₘₐₓ/cm⁻¹): 3309, 2960, 2920, 2851, 2094, 1715, 1648, 1571, 1464. HRMS (ESI): m/z=884.4736 [M+H]⁺ (calc. for C₄₈H₆₆N₇O₇S m/z=884.4739)

### Example 15

### N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide

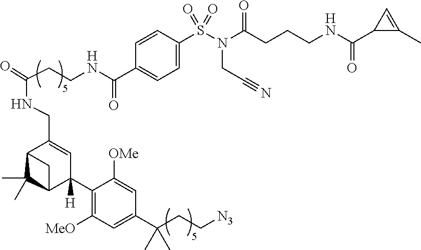

### Step b) tert-butyl 7-(4-(N-(4-((tert-butoxycarbonyl)amino)butanoyl)sulfamoyl)benzamido)heptanoate

To a solution of tert-butyl 7-(4-sulfamoylbenzamido)heptanoate (115 mg, 299 μmol, 1.0 equiv) in dry DMF (1.3 ml) was added Boc-GABA-OH (76 mg, 374 μmol, 1.25 equiv, CAS RN 57294-38-9), EDCI·HCl (86 mg, 449 μmol, 1.5 equiv), i-Pr₂NEt (260 μL, 1.50 mmol, 5.0 equiv), DMAP (11 mg, 90.1 μmol, 0.3 equiv) and the solution was stirred at rt overnight. The mixture was diluted with sat. aq. NH₄Cl solution (50 mL) and EtOAc (50 mL) and the phases were separated. The aqueous layer was extracted with EtOAc (2×50 mL), combined organic layers were washed with brine (25 mL), dried over MgSO₄, filtered and concentrated in vacuoo. The crude material was purified by flash column chromatography (SiO₂, 1% AcOH, 2-5% MeOH in CH₂Cl₂) to yield the product as a white solid (140 mg, 82%).

¹H NMR (400 MHz, CD₃OD) δ 8.66 (t, J=5.7 Hz, 1H), 8.13-8.03 (m, 2H), 8.01-7.90 (m, 2H), 3.44-3.35 (m, 2H), 2.96 (t, J=6.9 Hz, 2H), 2.24 (dt, J=9.3, 7.4 Hz, 4H), 1.71-1.55 (m, 6H), 1.45-1.37 (m, 4H).1.44 (s, 9H), 1.41 (s, 9H). ¹³C NMR (101 MHz, CD₃OD) δ 171.98, 167.14, 157.11, 141.96, 139.29, 127.93, 127.43, 79.96, 78.60, 39.72, 39.00, 34.92, 32.77, 28.81, 28.40, 27.35, 26.95, 26.32, 24.71, 24.40. IR (neat, vₘₐₓ/cm⁻¹): 3366, 3110, 2978, 2934, 2866, 1729, 1213, 1678, 1635, 1515, 1444, 1357. HRMS (ESI): m/z=592.2661 [M+Na]⁺ (calc. for C₂₇H₄₃N₃NaO₈S m/z=592.2663)

### Step e) 7-(4-(N-(4-(2-methylcycloprop-2-ene-1-carboxamido)butanoyl)sulfamoyl)benzamido)heptanoic acid

To a solution of tert-butyl 7-(4-(N-(4-((tert-butoxycarbonyl)amino)butanoyl)sulfamoyl)benzamido)heptanoate (38.0 mg, 66.7 μmol, 1.0 equiv) in CH₂Cl₂ (200 pl) was added TFA (200 pl) and the reaction was stirred at rt for 1 h. Volatiles were removed in vacuoo and residual TFA azeotropically removed with toluene (3×3 mL). The crude was dissolved in dry DMF (0.2 mL) and i-Pr₂NEt (58.1 μL, 334 μmol, 5.0 equiv) followed by 2,5-dioxopyrrolidin-1-yl-2-methylcycloprop-2-ene-1-carboxylate (15.6 mg, 80.0 μmol, 1.2 equiv) were added and the mixture was stirred at rt overnight. The mixture was diluted with 1M aq. HCl (30 mL) and EtOAc (30 mL) and the phases were separated. The aqueous layer was extracted with EtOAc (2×30 mL), combined organic layers were dried with MgSO₄, filtered and concentrated in vacuoo. The crude material was purified by flash column chromatography (1% AcOH, 5-10% MeOH in CH₂Cl₂) to yield the product as a white solid (31 mg, 85%).

¹H NMR (500 MHz, MeOD) δ 8.11-8.04 (m, 2H), 7.99-7.93 (m, 2H), 6.66-6.34 (m, 1H), 3.39 (t, J=7.2 Hz, 2H), 3.11 (td, J=6.9, 1.6 Hz, 2H), 2.30 (t, J=7.4 Hz, 2H), 2.24 (t, J=7.3 Hz, 2H), 2.12 (d, J=1.3 Hz, 3H), 1.96 (d, J=1.6 Hz, 1H), 1.73-1.58 (m, 6H), 1.41 (h, J=2.6 Hz, 4H). ¹³C NMR (126 MHz, MeOD) δ 179.39, 177.70, 173.64, 168.57, 143.49, 140.65, 129.30, 128.81, 113.99, 96.45, 41.12, 39.58, 34.89, 34.38, 30.22, 29.90, 27.75, 26.01, 25.53, 22.72, 10.40. IR (neat, vₘₐₓ/cm⁻¹): 3096, 2935, 2861, 2504, 1711, 1632, 1571, 1459, 1346, 1178. HRMS (ESI): m/z=494.1951 [M+H]⁺ (calc. for C₂₃H₃₂N₃O₇S m/z=494.1955)

### Step f) N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide

To a solution of 7-(4-(N-(4-(2-methylcycloprop-2-ene-1-carboxamido)butanoyl)sulfamoyl)benzamido)heptanoic acid (2.7 mg, 5.4 μmol, 1.25 equiv) in anhydrous DMF (50 μL) was added HATU (2.1 mg, 5.4 μmol, 1.25 equiv) and i-Pr₂NEt (3.1 μL, 17 μmol, 4.0 equiv) and the reaction mixture was stirred at rt for 5 min followed by addition of ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (2.0 mg, 4.3 μmol, 1.0 equiv) in anhydrous CH₂Cl₂ (25 μL). The reaction mixture was stirred at rt for 1 h and concentrated in vacuoo. The crude product was roughly purified by preparative TLC (SiO₂, 1% AcOH, 6% MeOH in CH₂Cl₂) to afford the intermediate as a colourless waxy solid which was immediately redissolved in DMF (50 μL). 2-iodoacetonitrile (2.0 μL, 27 μmol, 6.3 equiv) and i-Pr₂NEt (2.3 μL, 13 μmol, 3.0 equiv) were added and the off yellow mixture was stirred overnight at rt. Volatiles were removed in vacuoo and the crude was purified by preparative TLC (SiO₂, 5-7% MeOH in CH₂Cl₂) to yield the final compound as a white amorphous solid (2.4 mg, 56%).

¹H NMR (600 MHz, CD₂Cl₂) δ 8.06-7.99 (m, 2H), 7.99-7.91 (m, 2H), 7.12 (t, J=5.2 Hz, 1H), 6.49 (s, 2H), 6.46-6.39 (m, 1H), 5.61-5.55 (m, 1H), 5.48-5.40 (m, 2H), 4.77 (s, 2H), 3.95 (t, J=2.4 Hz, 1H), 3.87-3.74 (m, 2H), 3.72 (s, 6H), 3.43-3.38 (m, 2H), 3.22 (t, J=7.0 Hz, 2H), 3.05 (q, J=6.6 Hz, 2H), 2.65 (td, J=7.1, 0.9 Hz, 2H), 2.21-2.14 (m, 3H), 2.13 (d, J=1.3 Hz, 3H), 2.10-2.05 (m, 1H), 2.02 (tt, J=6.0, 1.8 Hz, 1H), 1.90 (d, J=1.6 Hz, 1H), 1.71-1.48 (m, 9H), 1.45-1.35 (m, 4H), 1.34-1.22 (m, 6H), 1.28 (s, 3H), 1.26 (s, 6H), 1.13-1.07 (m, 2H), 0.95 (s, 3H). ¹³C NMR (151 MHz, CD₂Cl₂) δ 176.72, 173.04, 172.43, 166.39, 159.02, 150.04, 142.17, 140.40, 139.21, 129.21, 128.64, 128.40, 128.02, 124.01, 118.01, 115.45, 114.59, 103.31, 96.58, 56.26, 52.06, 47.98, 44.95, 44.77, 40.55, 38.85, 38.49, 38.04, 37.09, 34.09, 33.77, 30.39, 30.22, 29.69, 29.37, 29.28, 29.19, 28.11, 27.16, 27.02, 26.59, 26.16, 25.29, 25.15, 22.86, 21.31, 14.45, 10.95. IR (neat, vₘₐₓ/cm⁻¹) 3301, 3074, 2923, 2852, 2095, 1717, 1642, 1605, 1571, 1544, 1463, 1410, 1365. HRMS (ESI): m/z=969.5260 [M+H]⁺ (calc. for C₅₂H₇₃N₈O₈S m/z=969.5267)

### Example 16

### N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo [c][1,2,5]selenadiazol-4-yl)oxy)heptanamide

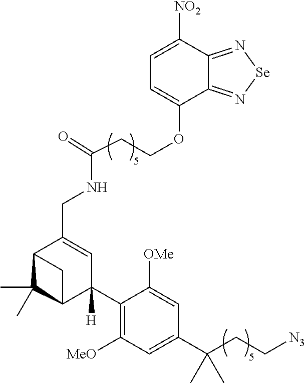

### Step a) tert-butyl 7-((7-nitrobenzo[c][1,2,5]selenadiazol-4-yl)oxy)heptanoate

Sodium hydride (60% suspension in oil, 4.2 mg, 104 μmol, 1.2 equiv) was suspended in anhydrous THF (0.35 mL) at 0° C. tert-butyl 7-hydroxyheptanoate (17.5 mg, 86.5 μmol, 1.0 equiv, CAS RN 86013-78-7) was added and the solution was stirred for 15 min. Subsequently, 4-fluoro-7-nitrobenzo[c][1,2,5]selenadiazole (32.0 mg, 130 μmol, 1.5 equiv, CAS RN 2351940-09-3) was added, the solution was warmed up to rt. and stirred overnight. The reaction mixture was quenched by addition of aq. sat. NH₄Cl(15 mL) and extracted with CH₂Cl₂ (3×30 mL). Combined organic extracts were dried with MgSO₄, filtered and concentrated in vacuoo. The crude oil was purified by flash column chromatography (SiO₂, 2% MeOH in CH₂Cl₂) to afford the product as a yellow solid (11.5 mg, 31%).

¹H NMR (500 MHz, CDCl₃) δ 8.65 (d, J=8.5 Hz, 1H), 6.71 (d, J=8.6 Hz, 1H), 4.33 (t, J=6.6 Hz, 2H), 2.23 (t, J=7.4 Hz, 2H), 2.09-1.97 (m, 2H), 1.69-1.54 (m, 4H), 1.47-1.41 (m, 2H), 1.43 (s, 9H). ¹³C NMR (126 MHz, CDCl₃) δ 173.18, 157.15, 154.09, 152.71, 134.87, 132.20, 102.88, 80.23, 70.48, 35.54, 28.85, 28.68, 28.26, 25.85, 25.02. IR (neat, vₘₐₓ/cm⁻¹): 2932, 2859, 1727, 1610, 1532, 1511, 1463, 1406, 1392, 1320, 1249. HRMS (ESI): m/z=452.0698 [M+Na]⁺ (calc. for Cl₇H₂₃N₃NaO₅Se m/z=452.0695)

### Step b) N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide

To a solution of tert-butyl 7-((7-nitrobenzo[c][1,2,5]selenadiazol-4-yl)oxy)heptanoate (2.9 mg, 6.8 μmol, 1.0 equiv) in dry CH₂Cl₂ (200 μL) was added TFA (100 μL) and the resulting deep red reaction mixture was allowed to stir for 30 min. at rt. Then, the solvents were removed in vacuoo, the residue was re-dissolved in CH₂Cl₂ and co-evaporated (×3). To the residue dissolved in anhydrous DMF (100 μL) was added anhydrous i-Pr₂NEt (2.9 μL, 16.5 μmol, 3.0 equiv), HATU (2.6 mg, 6.8 μmol, 1.25 eq.) and the reaction mixture was stirred at rt. for 15 min. ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (2.5 mg, 5.4 μmol, 1.0 equiv) was added to the mixture in anhydrous CH₂Cl₂ (100 μL) and the reaction mixture was stirred at rt. for 45 min and subsequently quenched with sat. aq. NaHCO₃ (5 mL). The aqueous layer was extracted with CH₂Cl₂ (3×15 mL) and the combined organic extracts washed with brine (10 mL). The organic layer was dried with Na₂SO₄, filtered and concentrated in vacuoo. The crude product was purified by preparative TLC (SiO₂, 2% MeOH in CH₂Cl₂) to afford the title product as a bright yellow solid (3.0 mg, 67%).

¹H NMR (500 MHz, CD₂Cl₂) δ 8.59 (d, J=8.4 Hz, 1H), 6.69 (d, J=8.6 Hz, 1H), 6.49 (s, 2H), 5.58 (dt, J=2.9, 1.5 Hz, 1H), 5.40 (t, J=5.4 Hz, 1H), 4.30 (t, J=6.6 Hz, 2H), 3.95 (t, J=2.3 Hz, 1H), 3.89-3.75 (m, 2H), 3.72 (s, 6H), 3.21 (t, J=7.0 Hz, 2H), 2.21-2.13 (m, 3H), 2.08 (td, J=5.6, 1.4 Hz, 1H), 2.04-1.93 (m, 3H), 1.74-1.63 (m, 3H), 1.62-1.42 (m, 8H), 1.37-1.21 (m, 4H), 1.27 (s, 3H), 1.26 (s, 6H), 1.17-1.06 (m, 2H), 0.94 (s, 3H). ¹³C NMR (126 MHz, CD₂Cl₂) δ 172.58, 158.86, 157.51, 149.88, 139.10, 132.34, 123.76, 117.84, 103.16, 103.13, 70.67, 56.09, 51.89, 47.81, 44.78, 44.61, 44.57, 41.07, 38.32, 37.87, 37.01, 30.23, 29.24, 29.20, 29.11, 28.94, 27.95, 26.99, 26.42, 26.10, 26.03, 24.99, 21.14. IR (neat, vₘₐₓ/cm⁻¹) 3309, 2927, 2856, 2094, 1649, 1607, 1571, 1523, 1463, 1410, 1324, 1294, 1239. HRMS (ESI): m/z=832.3280 [M+Na]⁺ (calc. for C₄₀H₅₅N₇NaO₆Se m/z=832.3275)

### Example 17

### N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)thio)octanamide

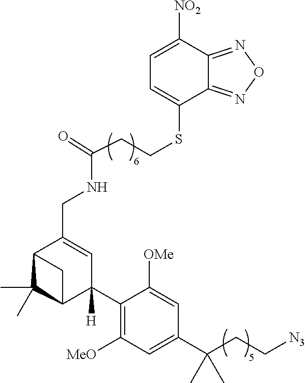

### Step a) 8-((7-nitrobenzo[c][1,2,5]oxadiazol-4-yl)thio)octanoic acid

To 8-sulfanylheptanoic acid (150 mg, 851 μmol, 1.0 equiv, CAS RN 74328-61-3) in a mixture of EtOH (1.5 mL) and water (4.5 mL) was added NBD-Cl (255 mg, 1.28 mmol, 1.5 equiv, CAS RN 10199-89-0), followed by pyridine (343 μL, 4.26 mmol, 5.0 equiv). The mixture gradually turned black and was vigorously stirred for 16 h at rt. The mixture was partitioned between EtOAc (150 mL) and aq. 1M HCl (150 mL), the layers were separated and the aqueous layer was extracted with EtOAc (2×150 mL). Combined organic fractions were dried over MgSO₄, filtered and concentrated in vacuoo. The crude product was adsorbed onto silica and purified by flash column chromatography (SiO₂, 30-50% EtOAc, 1% AcOH, in hexanes) to yield the product as a murky brown solid (182 mg, 63%).

¹H NMR (400 MHz, DMSO) δ 11.99 (s, 1H), 8.56 (d, J=8.0 Hz, 1H), 7.50 (d, J=8.1 Hz, 1H), 3.41-3.30 (m, 2H), 2.20 (t, J=7.3 Hz, 2H), 1.84-1.70 (m, 2H), 1.57-1.41 (m, 4H), 1.39-1.23 (m, 4H). ¹³C NMR (101 MHz, DMSO) δ 174.52, 149.21, 142.72, 140.04, 132.38, 132.12, 122.17, 33.61, 30.58, 28.39, 28.19, 28.08, 27.39, 24.41. IR (neat, vₘₐₓ/cm⁻¹): 3031, 2936, 2853, 1697, 1615, 1513, 1433, 1364, 1334, 1303. HRMS (ESI): m/z=340.0969 [M+H]⁺ (calc. for C₁₄H₁₈N₃O₅S m/z=340.0962)

### Step b) N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)thio)octanamide

To a solution of 8-((7-nitrobenzo[c][1,2,5]oxadiazol-4-yl)thio)octanoic acid (2.7 mg, 7.9 μmol, 1.2 equiv) in anhydrous DMF (50 μL) at 0° C. was added HATU (3.0 mg, 7.9 μmol, 1.2 equiv) and i-Pr₂NEt (3.5 μL, 20 μmol, 3.0 equiv) and the reaction mixture was stirred at 0° C. for 10 min. before being added to ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (3.0 mg, 6.5 μmol, 1.0 equiv) in anhydrous CH₂Cl₂ (50 μL). The ice bath was removed and the reaction mixture was stirred at rt. for 1 h, concentrated in vacuoo and purified by preparative TLC (SiO₂, 70% EtOAc in hexanes) to yield the final product as an orange solid (1.9 mg, 37%).

¹H NMR (500 MHz, CD₂Cl₂) δ 8.41 (d, J=7.9 Hz, 1H), 7.15 (d, J=7.9 Hz, 1H), 6.49 (s, 2H), 5.66-5.52 (m, 1H), 5.37 (t, J=5.7 Hz, 1H), 3.99-3.91 (m, 1H), 3.89-3.76 (m, 2H), 3.72 (s, 6H), 3.24 (t, J=7.4 Hz, 2H), 3.22 (t, J=7.0 Hz, 2H), 2.19-2.13 (m, 3H), 2.08 (td, J=5.7, 1.4 Hz, 1H), 2.04-1.99 (m, 1H), 1.89-1.78 (m, 2H), 1.69-1.49 (m, 7H), 1.44-1.21 (m, 1OH), 1.28 (s, 3H), 1.26 (s, 6H), 1.17-1.05 (m, 2H), 0.95 (s, 3H). ¹³C NMR (126 MHz, CD₂Cl₂) δ 172.25, 158.44, 149.47, 138.66, 131.04, 123.35, 120.44, 117.42, 102.71, 55.66, 51.48, 47.40, 44.37, 44.15, 40.65, 37.91, 37.45, 36.66, 31.70, 29.81, 29.68, 28.79, 28.77, 28.70, 28.64, 27.75, 27.51, 26.58, 26.00, 25.64, 24.57, 20.71. IR (neat, vₘₐₓ/cm⁻¹) 3316, 2929, 2857, 2094, 1725, 1648, 1571, 1522, 1508, 1411, 1362, 1328. HRMS (ESI): m/z=798.3969 [M+Na]⁺ (calc. for C₄₁H₅₇N₇NaO₆S m/z=798.3983)

### Example 18

### N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo [c][1,2,5]oxadiazol-4-yl)sulfinyl)octanamide

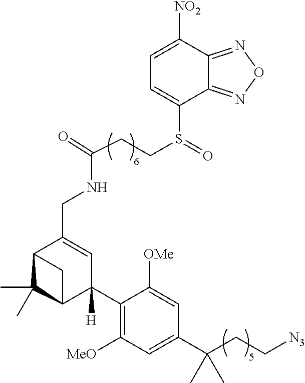

### Step c) 8-((7-nitrobenzo[c][1,2,5]oxadiazol-4-yl)sulfinyl)octanoic acid

A solution of mCPBA in dry CH₂Cl₂ (0.15M, 1.08 mL, 161 μmol, 1.5 equiv) was added dropwise at 0° C. to a solution of 8-((7-nitrobenzo[c][1,2,5]oxadiazol-4-yl)thio)octanoic acid (35 mg, 108 μmol, 1.0 equiv) in dry CH₂Cl₂ (0.5 mL). The resulting mixture was allowed to reach rt and stirred for 2 h. The mixture was directly purified by flash column chromatography (30-40% EtOAc, 1% AcOH in hexanes) to yield the product as a murky green solid (22.8 mg, 62%).

¹H NMR (400 MHz, CD₂Cl₂) δ 8.64 (d, J=7.5 Hz, 1H), 8.13 (d, J=7.5 Hz, 1H), 3.38 (ddd, J=13.5, 9.6, 6.0 Hz, 1H), 3.12 (ddd, J=13.5, 10.0, 4.6 Hz, 1H), 2.31 (t, J=7.5 Hz, 2H), 2.04-1.88 (m, 1H), 1.66-1.24 (m, 9H). ¹³C NMR (101 MHz, CD₂Cl₂) δ 178.69, 146.08, 143.38, 142.97, 137.92, 129.67, 128.25, 54.12, 33.62, 28.65, 28.59, 28.18, 24.43, 21.63. IR (neat, vₘₐₓ/cm⁻¹): 3096, 2930, 2859, 1706, 1548, 1534, 1464, 1407, 1368, 1340. HRMS (ESI): m/z=378.0730 [M+Na]⁺ (calc. for C₁₄H₁₇N₃NaO₆S m/z=378.0730)

### Step b) N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)sulfinyl)octanamide

To a solution of 8-((7-nitrobenzo[c][1,2,5]oxadiazol-4-yl)sulfinyl)octanoic acid (2.4 mg, 6.8 μmol, 1.25 equiv) in anhydrous DMF (50 μL) at 0° C. was added HATU (2.6 mg, 6.8 μmol, 1.25 equiv) and i-Pr₂NEt (2.9 μL, 17 μmol, 3.0 equiv) and the reaction mixture was stirred at 0° C. for 10 min. before being added to ((1S,4S,5S)-4-(4-(8-azido-2-methyloctan-2-yl)-2,6-dimethoxyphenyl)-6,6-dimethylbicyclo[3.1.1]hept-2-en-2-yl)methanamine (2.5 mg, 5.5 μmol, 1.0 equiv.) in anhydrous CH₂Cl₂ (50 μL). The ice bath was removed and the reaction mixture was stirred at rt. for 30 min., concentrated in vacuoo and purified by preparative TLC (SiO₂, 80% EtOAc in hexanes) to yield the final product as a brown solid (1.5 mg, 35%).

¹H NMR (500 MHz, CD₂Cl₂) 8.62 (d, J=7.5 Hz, 1H), 8.11 (d, J=7.5 Hz, 1H), 6.49 (s, 2H), 5.60-5.55 (m, 1H), 3.97-3.91 (m, 1H), 3.87-3.75 (m, 2H), 3.72 (s, 6H), 3.41-3.31 (m, 1H), 3.22 (t, J=7.0 Hz, 2H), 3.15-3.04 (m, 1H), 2.19-2.10 (m, 3H), 2.09-2.05 (m, 1H), 2.03-1.99 (m, 1H), 1.67 (dd, J=8.5, 1.5 Hz, 1H), 1.65-1.44 (m, 1OH), 1.37-1.22 (m, 8H), 1.27 (s, 3H), 1.26 (s, 6H), 1.16-1.05 (m, 2H), 0.94 (s, 3H). ¹³C NMR (126 MHz, CD₂Cl₂) δ158.44 (HMBC), 129.62, 123.37 (HSQC), 117.62 (HMBC), 102.71, 55.67, 54.22, 51.48, 47.40, 44.35, 44.14, 40.65, 37.91, 37.45, 36.62, 29.82, 29.68, 26.58, 26.00, 25.57, 24.57, 21.66, 20.71. IR (neat, Vmax/cm⁻¹) 2929, 2856, 2095, 1725, 1649, 1604, 1571, 1543, 1365, 1336, 1121. HRMS (ESI): m/z=792.4101 [M+H]⁺ (calc. for C₄₁H₅₈N₇O₇S m/z=792.4113)

### Example 19

### N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide

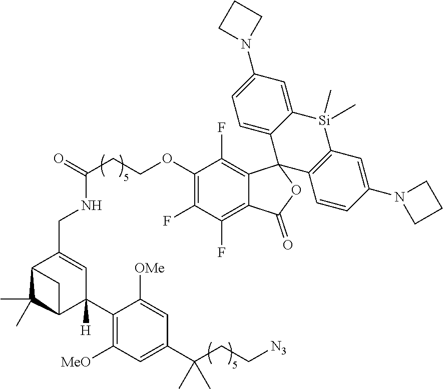

### Step a) tert-butyl 7-((3, 7-di(azetidin-1-yl)-4′,5′, 7′-trifluoro-5,5-dimethyl-3′-oxo-3′H,5H-spiro[dibenzo[b,e]siline-10,1′-isobenzofuran]-6′-yl)oxy)heptanoate

To a solution of tert-butyl 7-hydroxyheptanoate (1.7 mg, 8.3 μmol, 1.1 equiv, CAS RN 86013-78-7) in THF (0.1 mL) at 0° C. was added NaH (60% dispersion in mineral oil, 0.4 mg, 8.3 μmol, 1.1 equiv) resulting in a grey slurry. Then, 3,7-di(azetidin-1-yl)-4′,5′,6′,7′-tetrafluoro-5,5-dimethyl-3′H,5H-spiro[dibenzo[b,e]siline-10,1′-isobenzofuran]-3′-one (4.0 mg, 7.6 μmol, 1.0 equiv, prepared as described in Nat. Methods, 2020, 17, 815-821) in THF (0.1 mL) was added and the reaction mixture was allowed to stir at 0° C. for 1 h. The mixture was then diluted with water (5 mL) and CH₂Cl₂ (5 mL), the phases were separated, and the aqueous phase was extracted with CH₂Cl₂ (3×5 mL). The combined organic layers were dried over Na₂SO₄ and concentrated in vacuoo. Purification by preparative TLC (SiO₂, 7% MeOH in CH₂Cl₂) afforded the product (3.9 mg, 5.5 μmol, 72% yield) as a light blue solid.

¹H NMR (500 MHz, CD₂Cl₂) δ=6.74 (dd, J=8.6, 1.2 Hz, 2H), 6.67 (dd, J=2.7, 0.5 Hz, 2H), 6.34 (dd, J=8.7, 2.6 Hz, 2H), 4.40 (td, J=6.6, 1.0 Hz, 2H), 3.93 (t, J=7.3 Hz, 8H), 2.42-2.34 (m, 4H), 2.20 (t, J=7.2 Hz, 2H), 1.84 (dt, J=14.6, 6.7 Hz, 2H), 1.64-1.48 (m, 4H), 1.42 (s, 9H), 1.41-1.36 (m, 2H), 0.56 (s, 3H), 0.51 (s, 3H). ¹³C NMR (126 MHz, CD₂Cl₂) δ=173.43 (HMBC), 143.53 (HMBC), 130.48 (HMBC), 127.86, 116.39, 112.94, 80.20, 76.99, 52.92, 35.99, 30.42, 29.30, 28.39, 25.83, 25.56, 17.39, 1.33, 0.60. ¹⁹F NMR (471 MHz, CD₂Cl₂) δ=−143.02 (dd, J=21.9, 2.8 Hz), -147.22 (t, J=19.5 Hz), -147.76 (d, J=17.6 Hz). (decoupled) IR (neat, vₘₐₓ/cm⁻¹) 2930, 2854, 1768, 1728, 1595, 1484, 1405, 1365, 1306, 1265, 1164, 1103, 1005. HRMS (ESI): m/z=707.3123 [M+H]⁺ (calc. for C₃₉H₄₆F₃N₂O₅Si m/z=707.3123)

### Step b) N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3, 7-di(azetidin-1-yl)-4′,5′, 7′-trifluoro-5,5-dimethyl-3′-oxo-3′H,5H-spiro[dibenzo[b,e]siline-10,1′-isobenzofuran]-6′-yl)oxy)heptanamide

To a solution of tert-butyl 7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanoate (2.0 mg, 2.8 μmol, 1.0 equiv) in dry CH₂Cl₂ (200 μL) was added TFA (100 μL) and the resulting deep blue solution was allowed to stir for 1 h. Then, the solvents were concentrated in vacuoo, the residue was re-dissolved in CH₂CH₂ and co-evaporated (×3). To the crude product was then added i-Pr₂NEt (0.5 solution in DMF, 34.0 μL, 16.9 μmol, 6.0 equiv) and the resulting light blue solution was cooled to 0° C. Then, HATU (0.1 M solution in DMF, 34.0 μL, 3.4 μmol, 1.2 equiv) was added, the reaction was stirred at 0° C. for 15 min and ((1S,4S,5S)-4-(4-(8-azido-2-methyloctan-2-yl)-2,6-dimethoxyphenyl)-6,6-dimethylbicyclo[3.1.1]hept-2-en-2-yl)methanamine was added (0.05 M solution in dry CH₂Cl₂, 59.4 μL, 3.0 μmol, 1.05 equiv). The mixture was allowed to warm to ambient temperature and stirred for further 30 min. The reaction mixture was then diluted with sat. aq. NaHCO₃ (5 mL) and CH₂Cl₂ (5 mL). The phases were separated, the aqueous phase was extracted with CH₂Cl₂ (3×5 mL), dried over Na₂SO₄ and concentrated in vacuoo. Purification by preparative TLC (SiO₂, 2% MeOH in CH₂Cl₂) afforded the product (0.9 mg, 29%) as a blueish solid.

HRMS (ESI): m/z=1087.5695 [M+H]⁺ (calc. for C₆₂H₇₈F₃N₆O₆Si m/z=1087.5699)

1. A compound of formula (I) 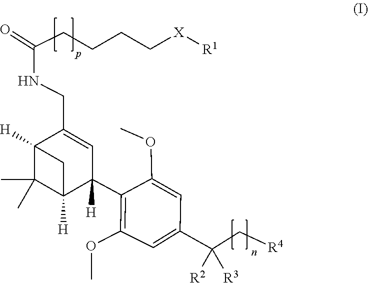
or a pharmaceutically acceptable salt thereof, wherein:
(i) R¹ 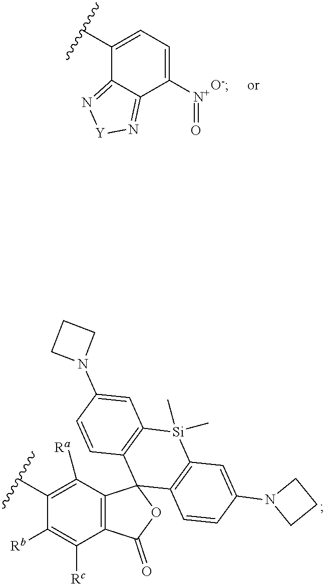
and
X is selected from O, S, and SO; or
(ii) R¹ is 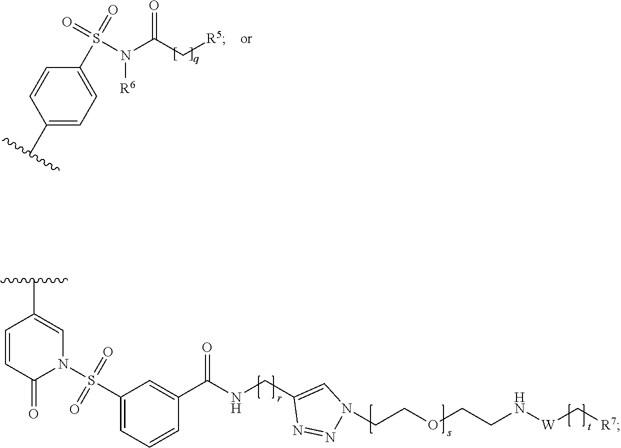
and
X is selected from O, S, SO, and NHCO;
R² and R³ are selected from hydrogen and C₁-C₄-alkyl; or
R² and R³, taken together with the carbon atom to which they are attached, form a C₃-C₅-cycloalkyl;
R⁴ is selected from hydrogen, halogen, cyano, and azide;
R⁵ is selected from: 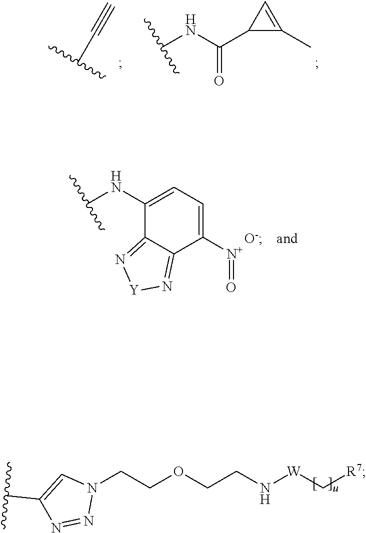
R⁶ is selected from: 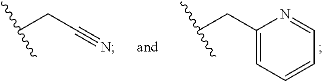
R⁷ is selected from: 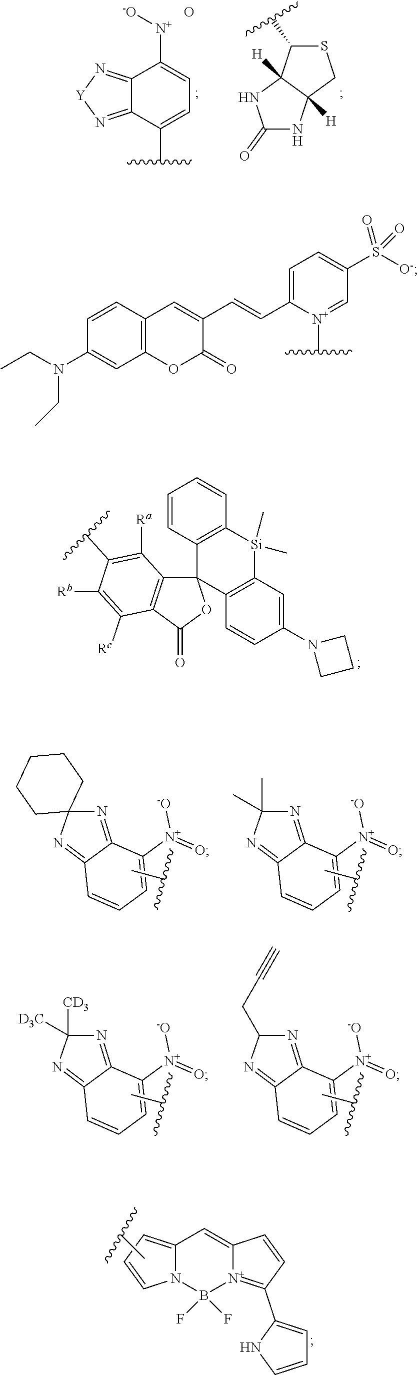 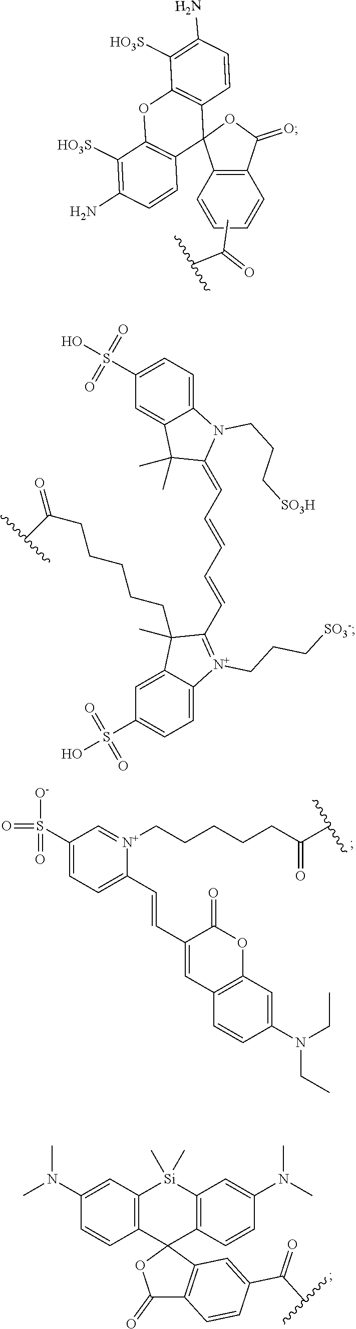 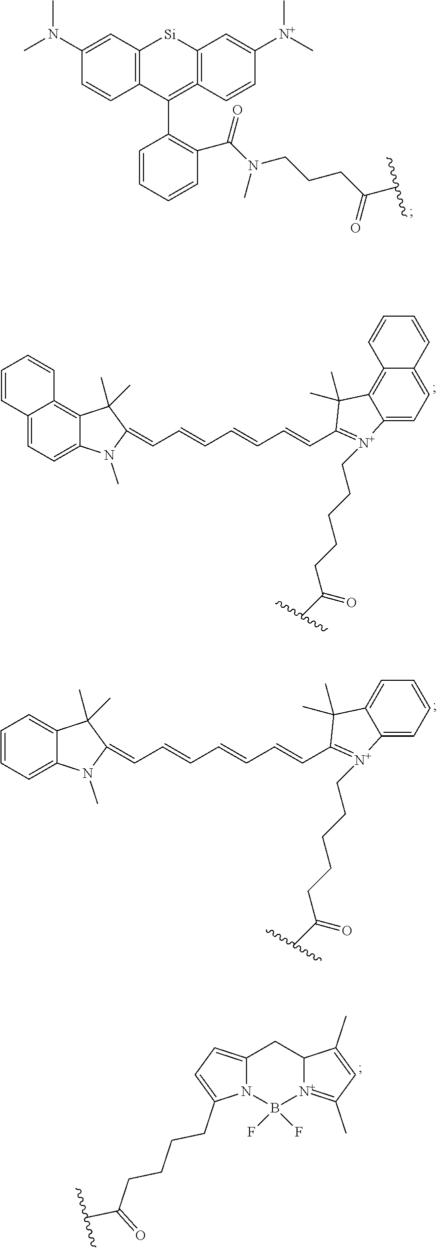 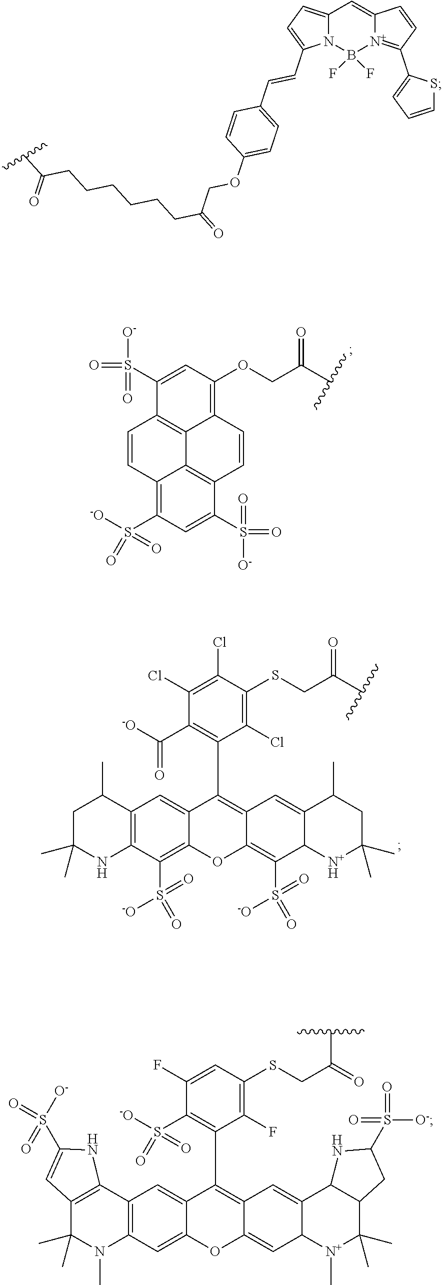 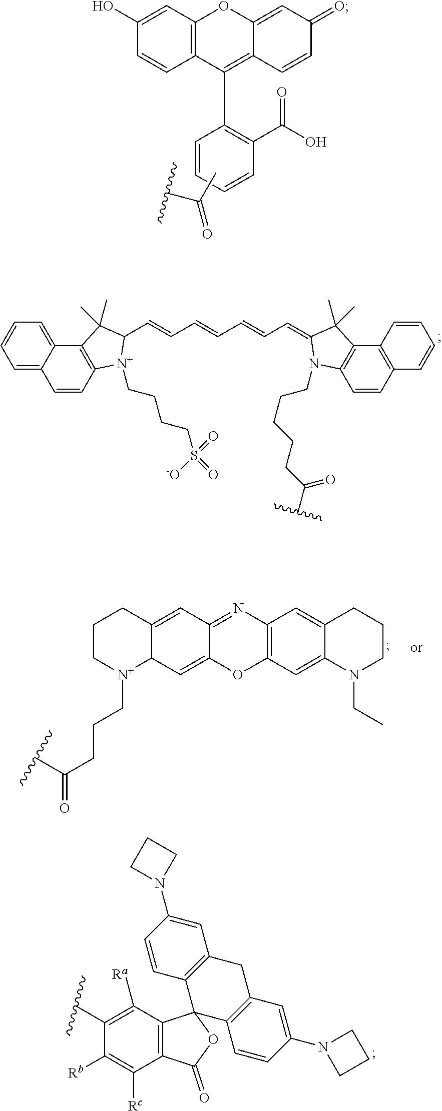
R^{a}, R^{b}, and R^{c} are each independently selected from hydrogen and halogen;
n is selected from 1, 2, 3, 4, 5 and 6;
p is selected from 1, 2, 3, 4, 5, 6, and 7;
q is selected from 1, 2, 3, 4, and 5;
r is selected from 1 and 2;
s is selected from 1, 2, 3, and 4;
t is selected from 0, 1, 2, 3, 4, and 5;
u is selected from 0, 1, 2, 3, 4, and 5;
W is carbonyl or absent;
Y is selected from Se, S, and O.

2. The compound of formula (I) according to claim 1, or a pharmaceutically acceptable salt thereof, wherein:
(i) R¹ is 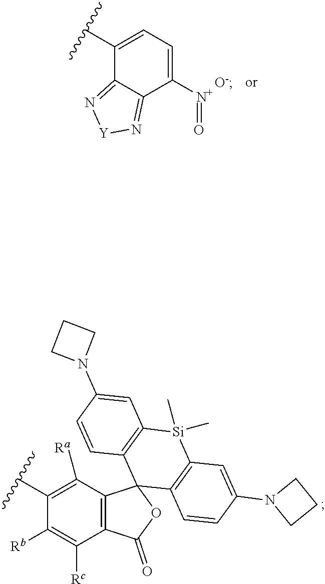
and
X is selected from O, S, and SO; or
(ii) R¹ is 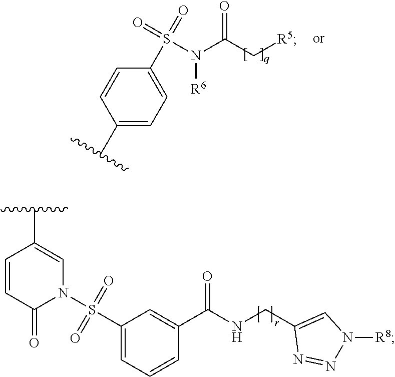
and
X is selected from O, S, SO, and NHCO;
R² and R³ are selected from hydrogen and C₁-C₄-alkyl; or
R² and R³, taken together with the carbon atom to which they are attached, form a C₃-C₅-cycloalkyl;
R⁴ is selected from hydrogen, halogen, cyano, and azide;
R⁵ is selected from: 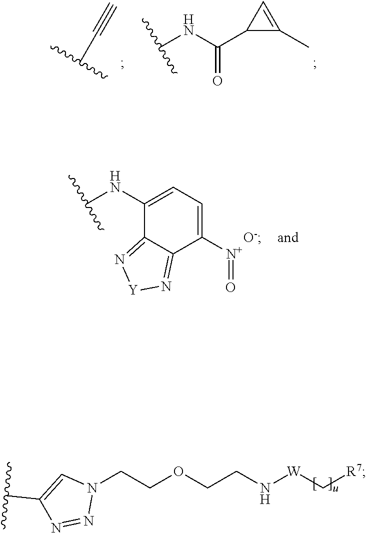
R⁶ is selected from: 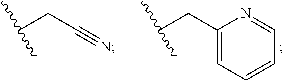
R⁷ is selected from: 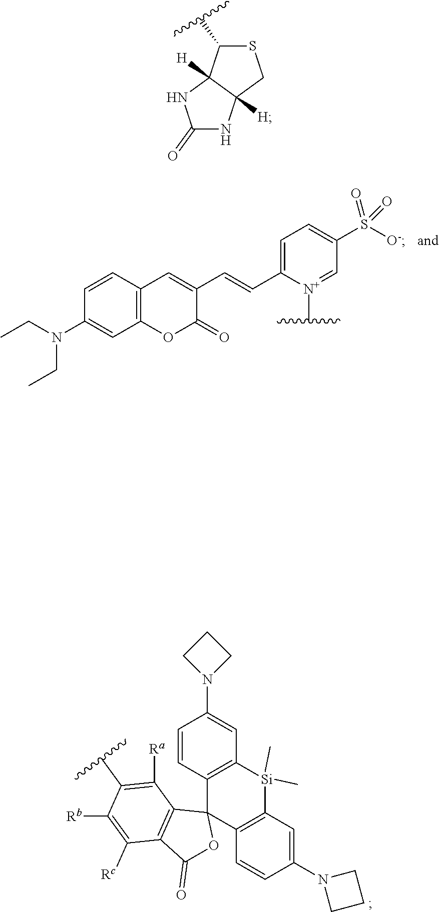
R⁸ is selected from: 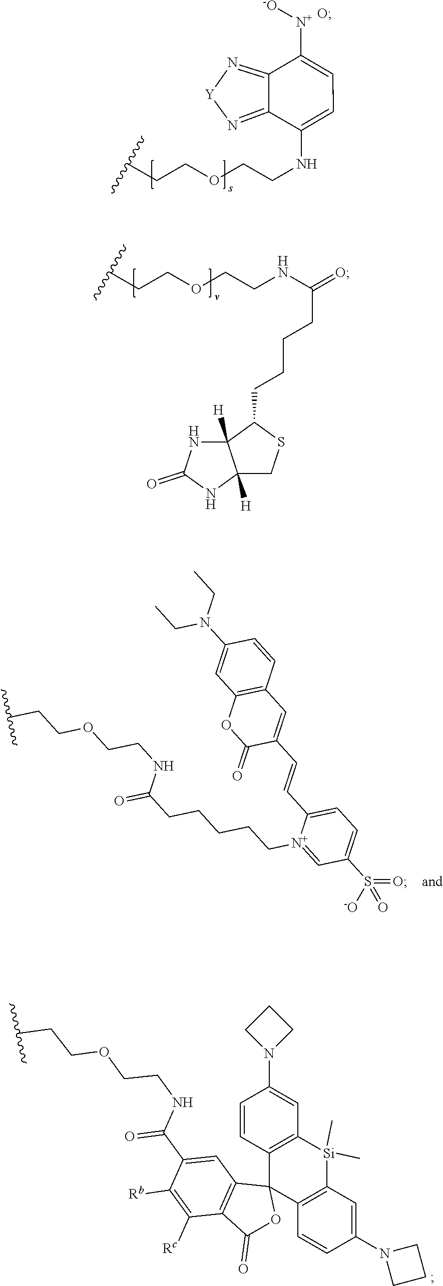
R^{a}, R^{b}, an R^{c} are each independently selected rom hydrogen an halogen;
n is selected from 1, 2, 3, 4, 5 and 6;
p is selected from 1, 2, 3, 4, 5, 6, and 7;
q is selected from 1, 2, 3, 4, and 5;
r is selected from 1 and 2;
s is selected from 1, 2, 3, and 4;
u is selected from 0, 1, 2, 3, 4, and 5;
v is selected from 1, 2, 3, and 4;
W is carbonyl or absent;
Y is selected from Se, S, and O.

3. The compound of formula (I) according to claim 1, or a pharmaceutically acceptable salt thereof, wherein:
(i) R¹ is 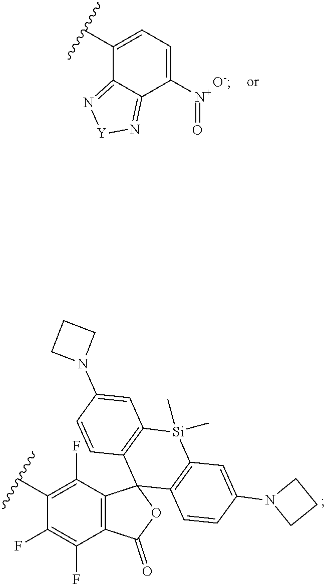
and
X is selected from O, S, and SO; or
(ii) R¹ is 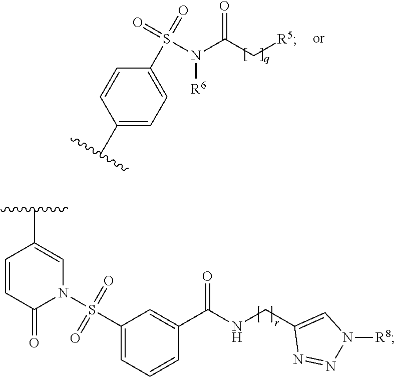
and
X is NHCO;
R⁵ is selected from: 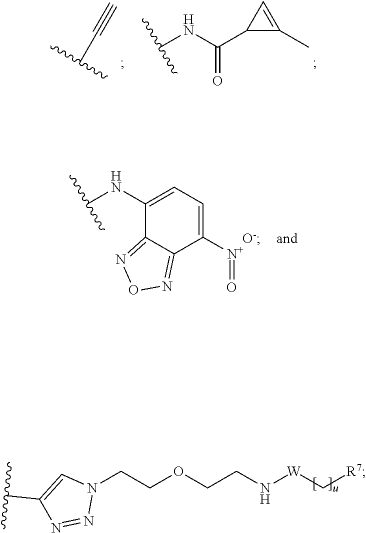
R⁶ is selected from: 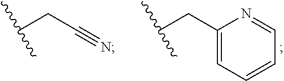
R⁷ is selected from: 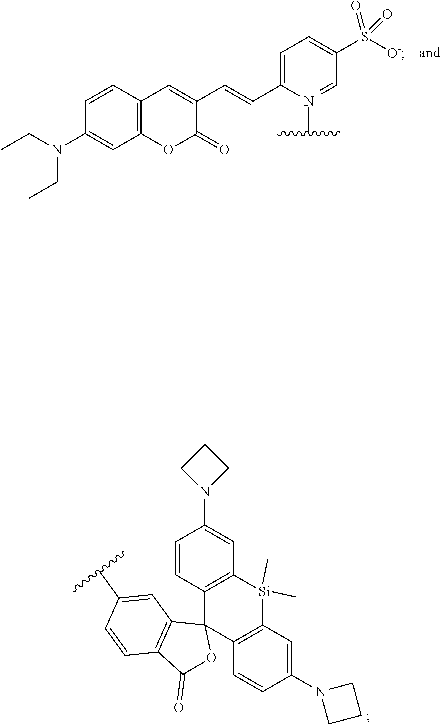
R⁸ is selected from: 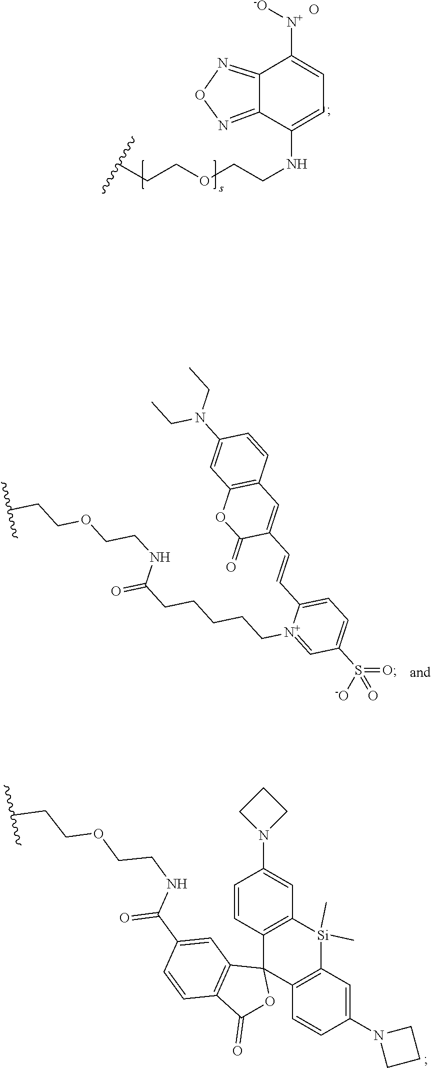
p is selected from 1, 3, 4, and 5;
q is selected from 2, 3, and 5;
r is selected from 1 and 2;
s is selected from 1 and 4;
u is selected from 0, 4, and 5;
W is carbonyl or absent;
Y is selected from Se and O.

4. The compound of formula (I) according to claim 3, or a pharmaceutically acceptable salt thereof, wherein:
(i) R¹ is 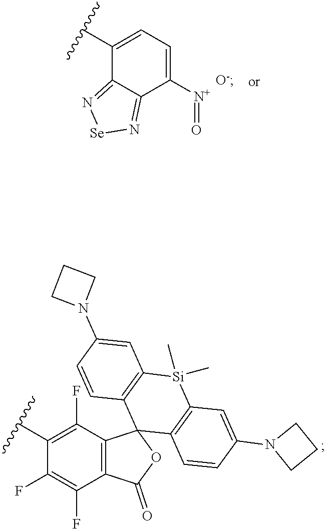
and
X is O; or
(ii) R¹ is 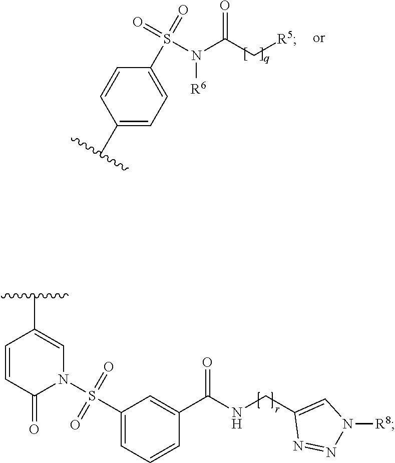
and
X is NHCO;
R⁵ is selected from: 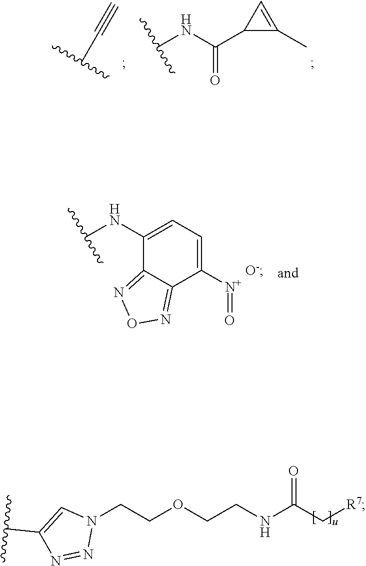
R⁶ is selected from: 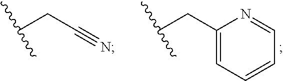
R⁷ is selected from: 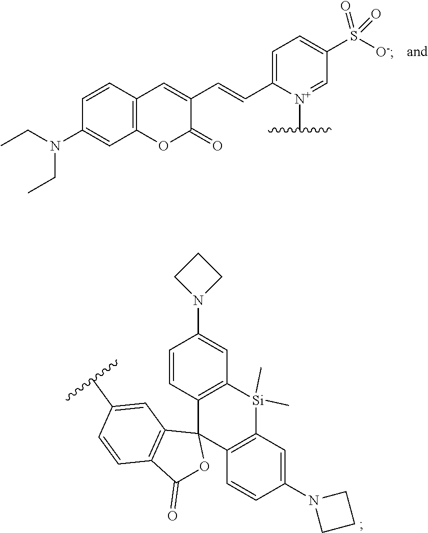
R⁸ is selected from: 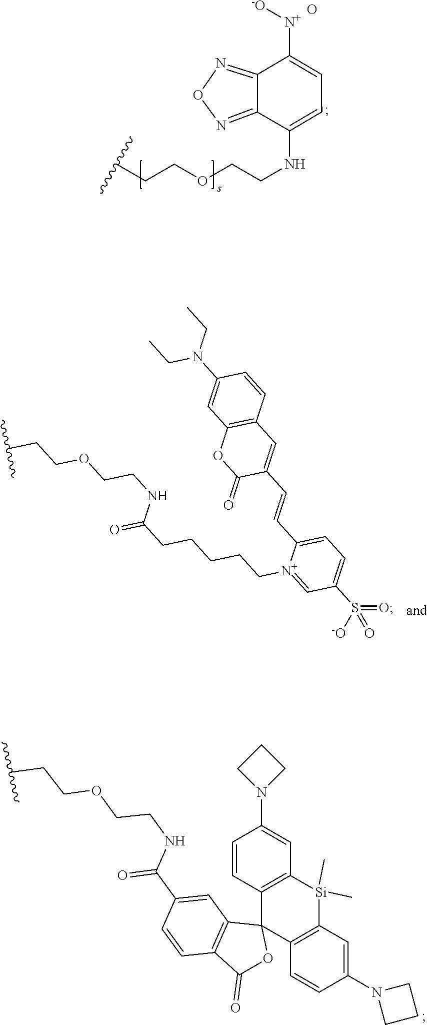
p is 3;
q is selected from 2, 3, and 5;
r is 1;
s is 1; and
u is 5.

5. The compound of formula (I) according to claim 4, or a pharmaceutically acceptable salt thereof, wherein:
R¹ is 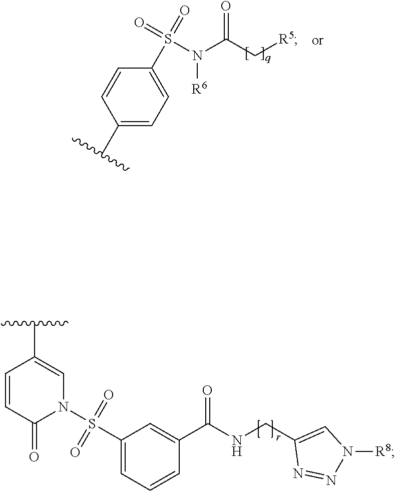
R⁵ is 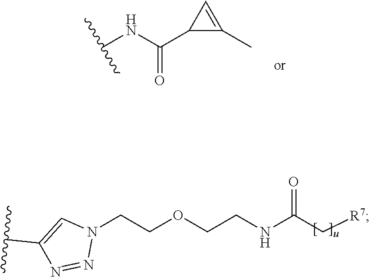
R⁶ is 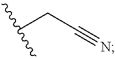 [SMILES: *CC#N]
R⁷ is 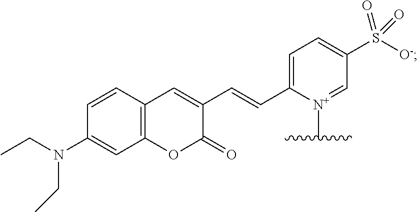
R⁸ is 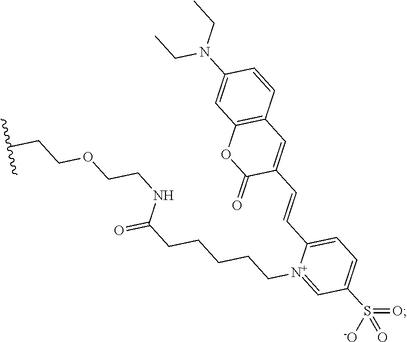
X is NHCO;
p is 3;
q is 2 or 3;
r is 1; and
u is 5.

6. The compound of formula (I) according to claim 1, or a pharmaceutically acceptable salt thereof, wherein:
R² and R³ are selected from hydrogen and C₁-C₄-alkyl;
R⁴ is hydrogen or azide; and
n is 6.

7. The compound of formula (I) according to claim 6, or a pharmaceutically acceptable salt thereof, wherein:
R² and R³ are both C₁-C₄-alkyl;
R⁴ is azide; and
n is 6.

8. The compound of formula (I) according to claim 7, or a pharmaceutically acceptable salt thereof, wherein:
R² and R³ are both methyl;
R⁴ is azide; and
n is 6.

9. The compound of formula (I) according to claim 1, or a pharmaceutically acceptable salt thereof, wherein:
(i) R¹ is 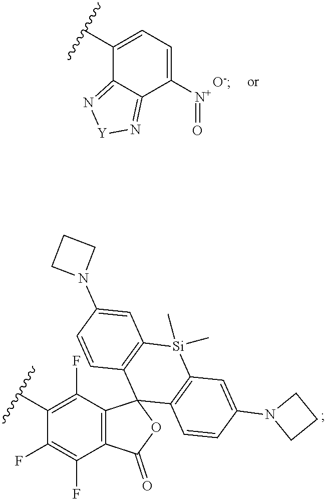
and
X is selected from O, S, and SO; or
(ii) R¹ is 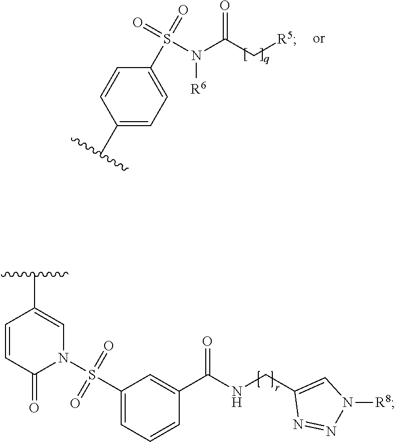
and
X is NHCO;
R² and R³ are selected from hydrogen and C₁-C₄-alkyl;
R⁴ is hydrogen or azide;
R⁵ is selected from: 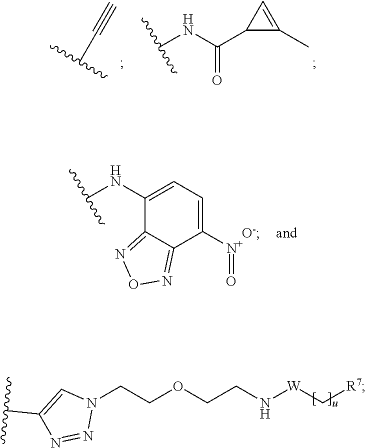
R⁶ is selected from: 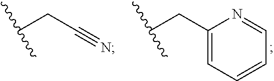
R⁷ is selected from: 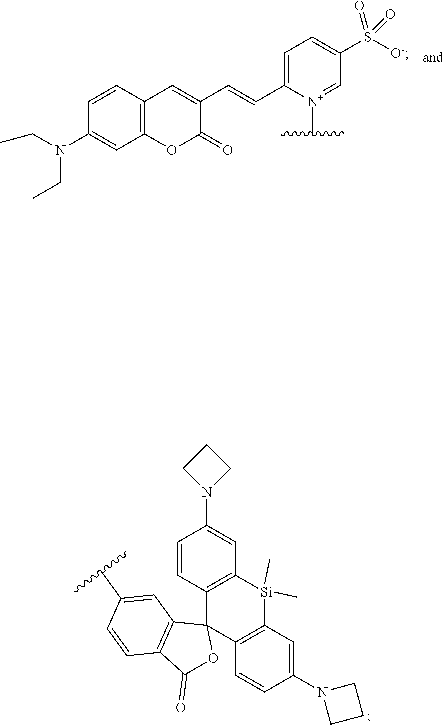
R⁸ is selected from: 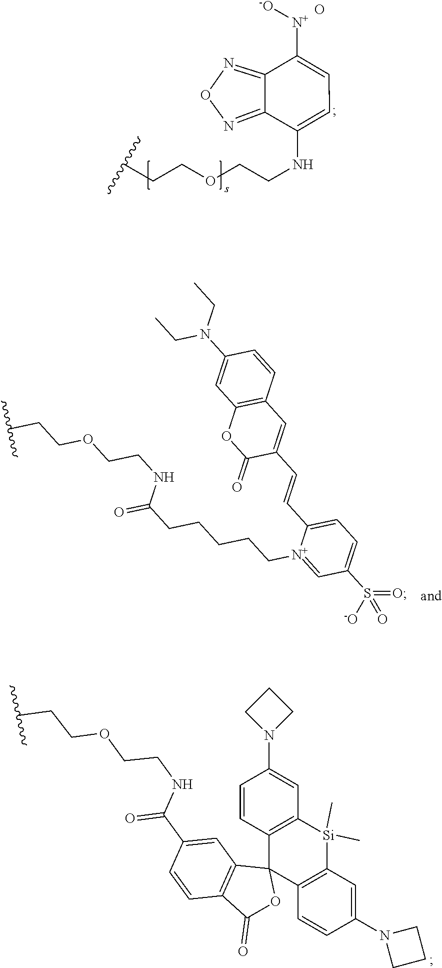
n is 6;
p is selected from 1, 3, 4, and 5;
q is selected from 2, 3, and 5;
r is selected from 1 and 2;
s is selected from 1 and 4;
u is selected from 0, 4, and 5;
W is carbonyl or absent;
Y is selected from Se and O.

10. The compound of formula (I) according to claim 9, or a pharmaceutically acceptable salt thereof, wherein:
(i) R¹ is 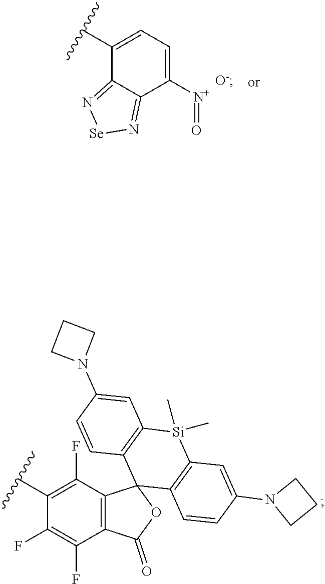
and
X is O; or
(ii) R¹ is 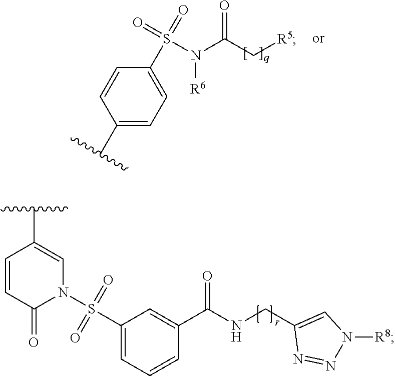
and
X is NHCO;
R² and R³ are both C₁-C₄-alkyl;
R⁴ is azide;
R⁵ is selected from: 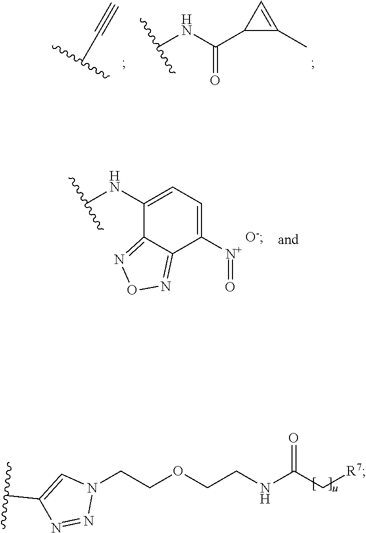
R⁶ is selected from: 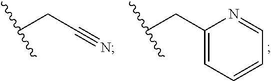
R⁷ is selected from: 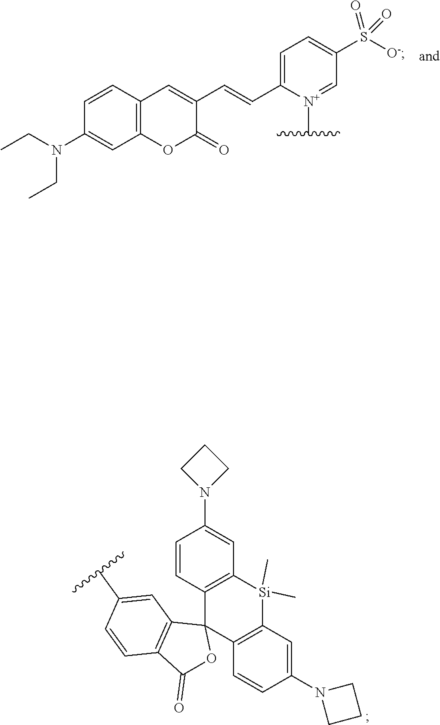
R⁸ is selected from: 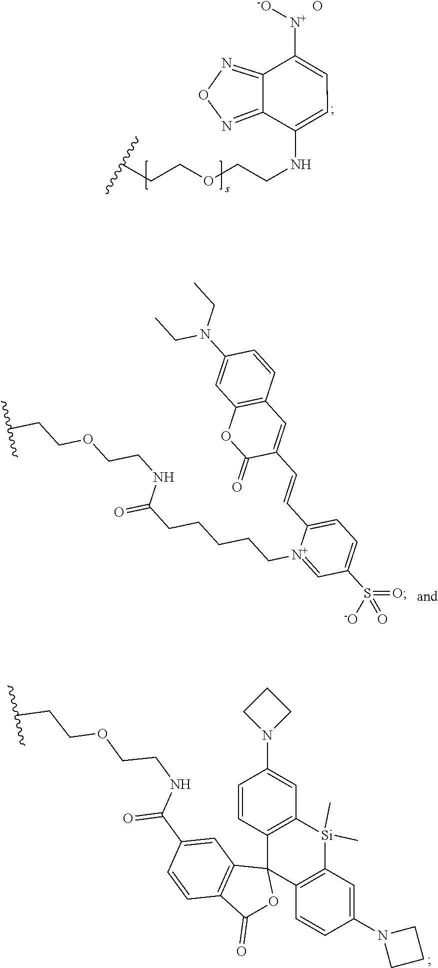
n is 6;
p is 3;
q is selected from 2, 3, and 5;
r is 1;
s is 1; and
u is 5.

11. The compound of formula (I) according to claim 10, or a pharmaceutically acceptable salt thereof, wherein:
R¹ is 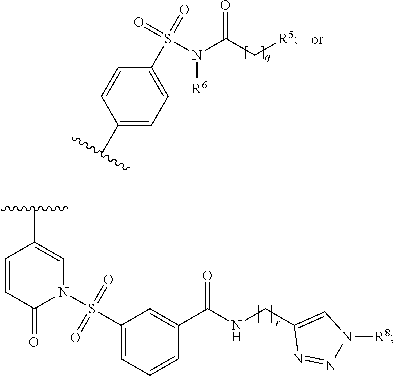
R² and R³ are both methyl;
R⁴ is azide;
R⁵ is 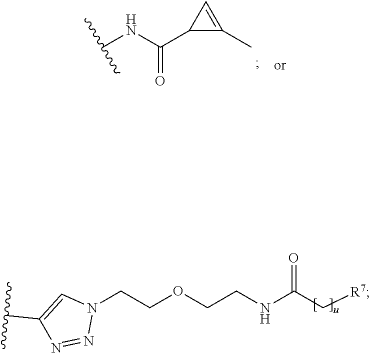
R⁶ is 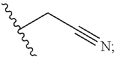 [SMILES: *CC#N]
R⁷ is 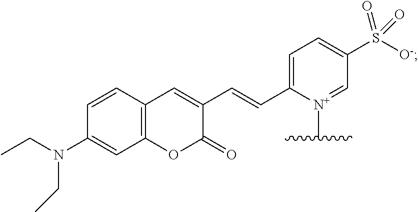
R⁸ is 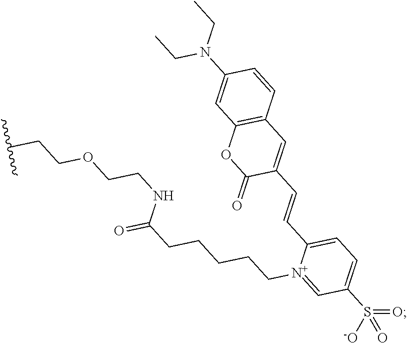
X is NHCO;
n is 6;
p is 3;
q is 2 or 3;
r is 1; and
u is 5.

12. The compound of formula (I) according to claim 1, or a pharmaceutically acceptable salt thereof, wherein the compound of formula (I) is selected from:
N-(9-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-9-oxononyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(5-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-5-oxopentyl)-1-((3-((2-(1-(2-(2-((7-nitrobenzo[c][1,2,5]oxadiazol-4-yl)amino)ethoxy)ethyl)-1H-1,2,3-triazol-4-yl)ethyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(14-((7-nitrobenzo[c][1,2,5]oxadiazol-4-yl)amino)-3,6,9,12-tetraoxatetradecyl)-1H-1,2,3-triazol-4-yl)methyl)carbamoyl)phenyl)sulfonyl)-6-oxo-1,6-dihydropyridine-3-carboxamide;
1-(6-((2-(2-(4-((3-((5-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-2-oxopyridin-1 (2H)-yl)sulfonyl)benzamido)methyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-1-((3-(((1-(2-(2-((7-nitrobenzo[c][1,2,5]oxadiazol-4-yl)amino)ethoxy)ethyl)-1H-1,2,3-triazol-4-yl)methyl)carbamoyl)phenyl)sulfonyl)-6-oxo-1,6-dihydropyridine-3-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(2,6-dimethoxy-4-(2-methyloctan-2-yl)phenyl)-6,6-dimethylbicyclo[3.1.1]hept-2-en-2-yl)methyl)amino)-7-oxoheptyl)-4-(N-(6-((7-nitrobenzo[c][1,2,5]oxadiazol-4-yl)amino)hexanoyl)-N-(pyridin-2-ylmethyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(6-((7-nitrobenzo[c][1,2,5]oxadiazol-4-yl)amino)hexanoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-1-((3-(((1-(2-(2-(3,7-di(azetidin-1-yl)-5,5-dimethyl-3′-oxo-3′H,5H-spiro[dibenzo[b,e]siline-10,1′-isobenzofuran]-6′-carboxamido)ethoxy)ethyl)-1H-1,2,3-triazol-4-yl)methyl)carbamoyl)phenyl)sulfonyl)-6-oxo-1,6-dihydropyridine-3-carboxamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((7-nitrobenzo[c][1,2,5]selenadiazol-4-yl)oxy)heptanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-((7-nitrobenzo[c][1,2,5]oxadiazol-4-yl)amino)ethoxy)ethyl)-1H-1,2,3-triazol-4-yl)propanoyl)sulfamoyl)benzamide;
1-(6-((2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate;
3,7-di(azetidin-1-yl)-N-(2-(2-(4-(3-((4-((7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)-5,5-dimethyl-3′-oxo-3′H,5H-spiro[dibenzo[b,e]siline-10,1′-isobenzofuran]-6′-carboxamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(pent-4-ynoyl)sulfamoyl)benzamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)thio)octanamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-8-((7-nitrobenzo[c][1,2,5]oxadiazol-4-yl)sulfinyl)octanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(4-(2-methylcycloprop-2-ene-1-carboxamido)butanoyl)sulfamoyl)benzamide;
N-(((1S,4S,5S)-4-(4-(8-azido-2-methyloctan-2-yl)-2,6-dimethoxyphenyl)-6,6-dimethylbicyclo[3.1.1]hept-2-en-2-yl)methyl)-7-((3,7-di(azetidin-1-yl)-4′,5′,7′-trifluoro-5,5-dimethyl-3′-oxo-3′H,5H-spiro[dibenzo[b,e]siline-10,1′-isobenzofuran]-6′-yl)oxy)heptanamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(2-(2-(5-((3aS,4S,6aR)-2-oxohexahydro-1H-thieno[3,4-d]imidazol-4-yl)pentanamido)ethoxy)ethyl)-1H-1,2,3-triazol-4-yl)propanoyl)sulfamoyl)benzamide;
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-4-(N-(cyanomethyl)-N-(3-(1-(16-oxo-20-((3aS,4S,6aR)-2-oxohexahydro-1H-thieno[3,4-d]imidazol-4-yl)-3,6,9,12-tetraoxa-15-azaicosyl)-1H-1,2,3-triazol-4-yl)propanoyl)sulfamoyl)benzamide; and
N-(7-((((1S,4S,5S)-4-(4-(8-azido-2-methyloctan-2-yl)-2,6-dimethoxyphenyl)-6,6-dimethylbicyclo[3.1.1]hept-2-en-2-yl)methyl)amino)-7-oxoheptyl)-6-oxo-1-((3-(((1-(2-(2-(5-((3aS,4S,6aR)-2-oxohexahydro-1H-thieno[3,4-d]imidazol-4-yl)pentanamido)ethoxy)ethyl)-1H-1,2,3-triazol-4-yl)methyl)carbamoyl)phenyl)sulfonyl)-1,6-dihydropyridine-3-carboxamide.

13. A process for manufacturing a compound of formula (I) according to claim 1, comprising:
(a) reacting an alkyne C or I 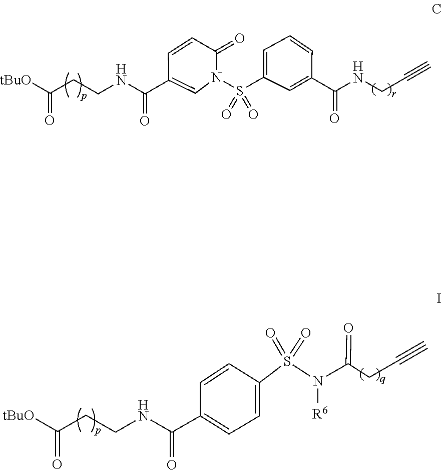
wherein R⁶, p, q, and r are as defined in claim 1, with an azide-substituted fluorophore M 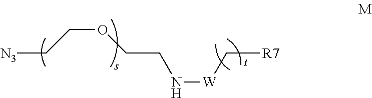
wherein W, R⁷, s, and t are as defined in claim 1;
to form a compound D or K respectively 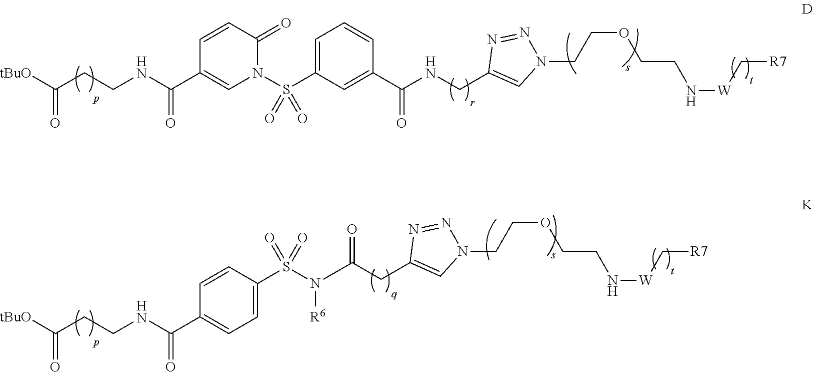
wherein W, R⁶, R⁷, p, q, r, and s are as defined in claim 1;
(b) reacting the compound D or K with an acid to afford compound D1 or K1 respectively 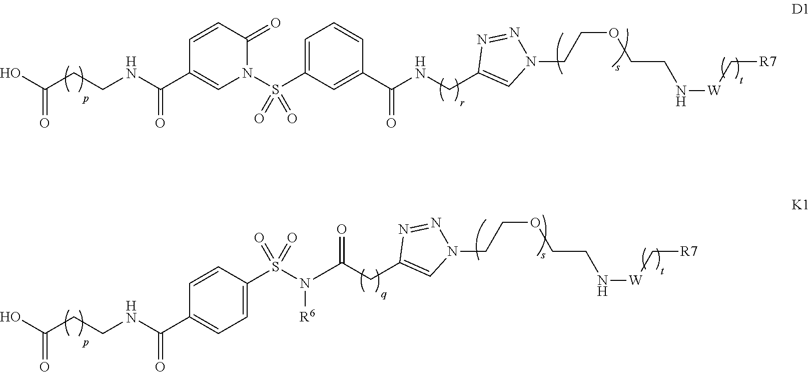
wherein W, R⁶, R⁷, p, q, r, and s are as defined in claim 1;
and
(c) reacting the compound D1 or K1 with amine N1, 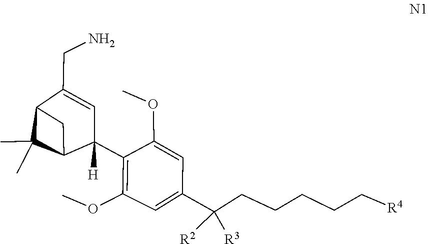
wherein R², R³, and R⁴ are as defined in claim 1;
in the presence of a coupling reagent and an acid/base buffer system;
to afford said compound of formula (I).

14. A compound selected from: 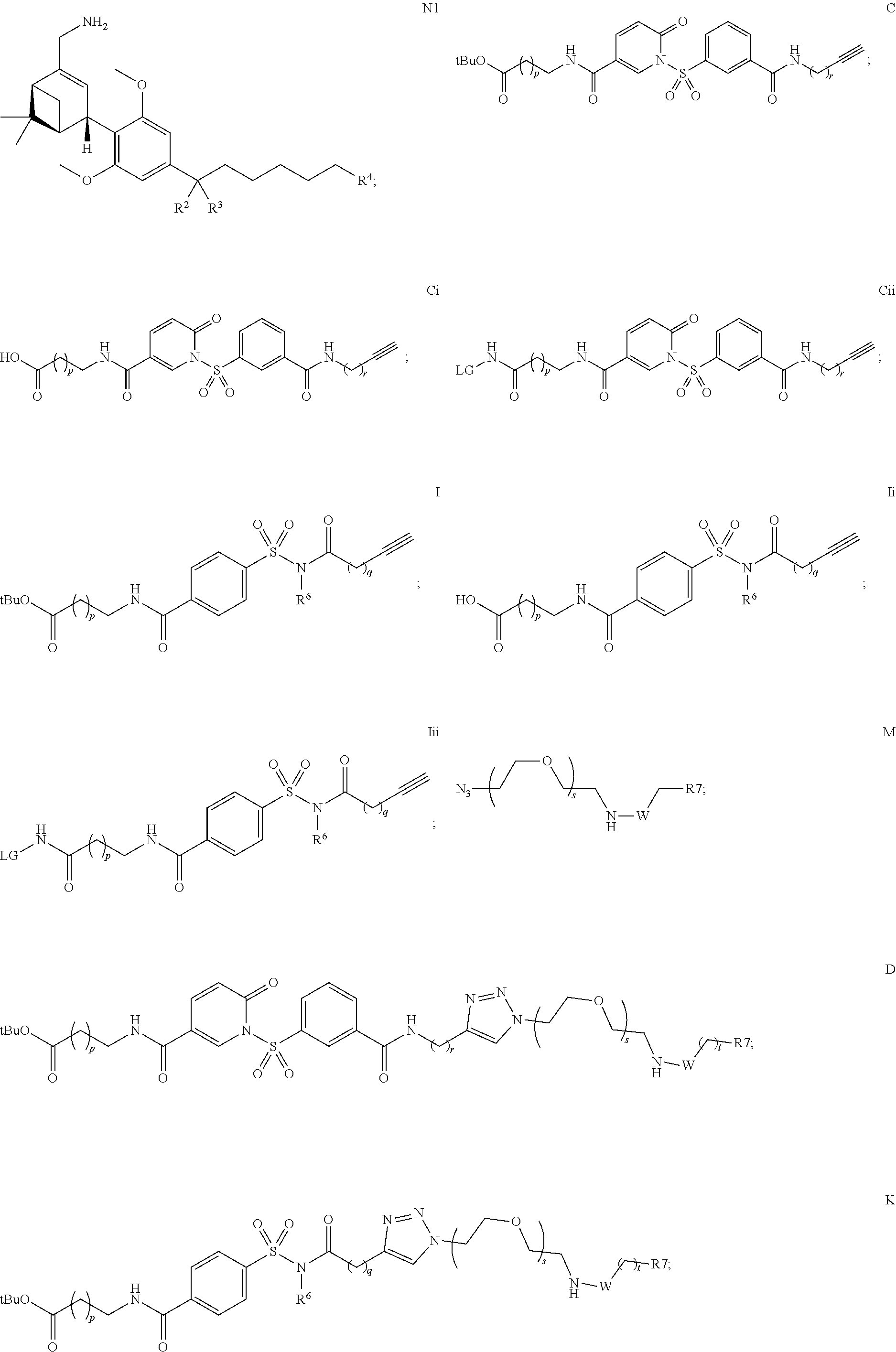 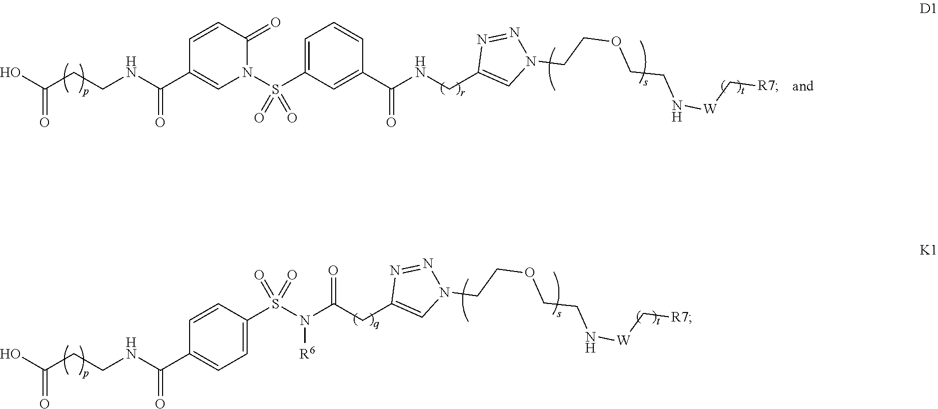
wherein R², R³, R⁴, R⁶, R⁷, p, q, r, s, t are as defined in claim 1 and LG is

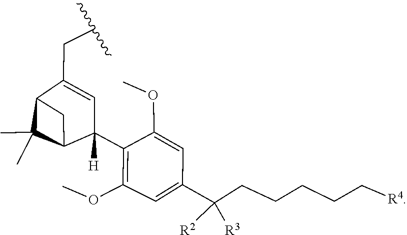

15. A process for manufacturing an amide of formula 1 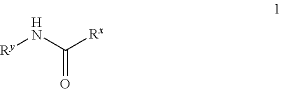
wherein R^{x} and R^{y} are both chemical moieties that are attached to the parent amide functional group through a carbon atom; and
wherein R^{x} comprises an N-sulfonyl pyridone (SP) motif i, or an N-acyl-N-alkyl sulfonamide (NASA) motif ii 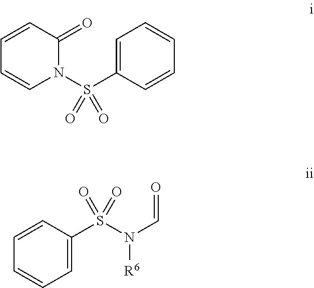
wherein R⁶ is as defined in claim 1;
comprising:
reacting a carboxylic acid 2 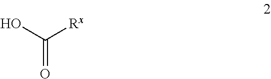
with an amine 3 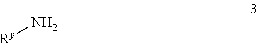
in the presence of a coupling reagent and an acid/base buffer system;
to afford said amide 1.

16. The process of claim 15, wherein the amide of formula 1 is a fluorescent probe P1 or P2 for a protein of interest, 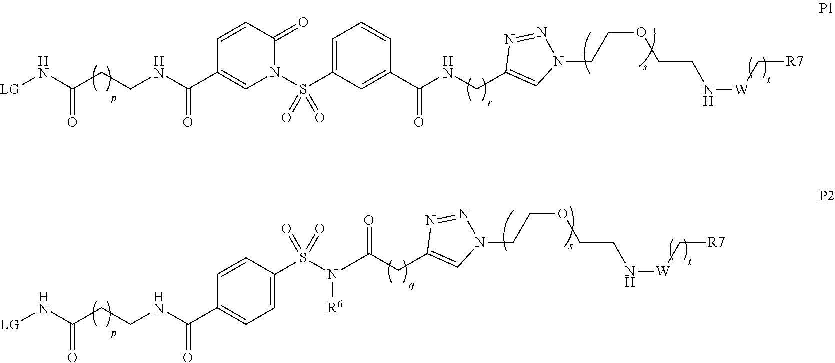
wherein LG is a ligand for the protein of interest, and W, R⁶, R⁷, p, q, r, and s are as defined in claim 1, comprising:
(a) reacting an alkyne C or I 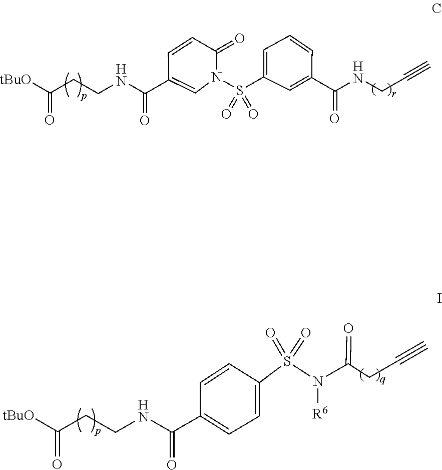
wherein R⁶, p, q, and r are as defined in claim 1, with an azide-substituted fluorophore M 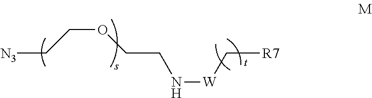
wherein W, R⁷, s, and t are as defined in claim 1;
to form a compound D or K respectively 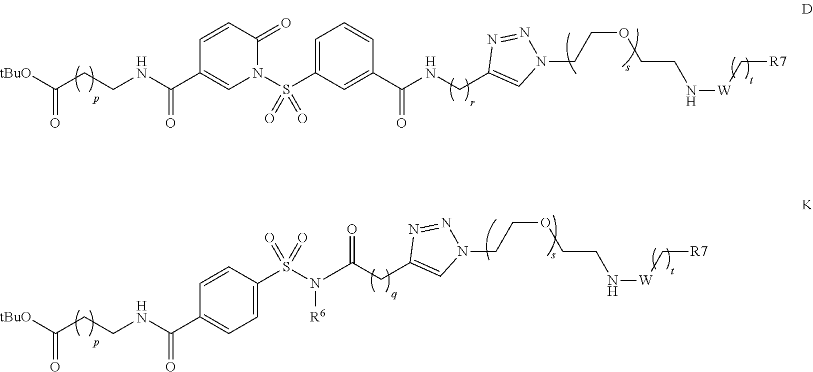
wherein W, R⁶, R⁷, p, q, r, and s are as defined in claim 1;
(b) reacting the compound D or K with an acid to afford compound D1 or K1 respectively 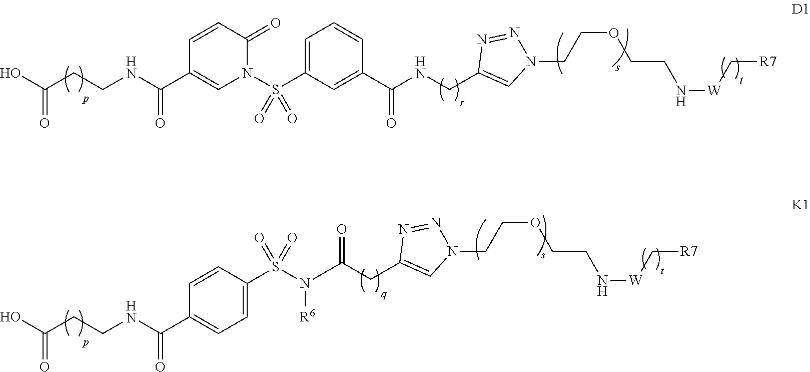
wherein W, R⁶, R⁷, p, q, r, and s are as defined in claim 1; and
(c) reacting the compound D1 or K₁ with an amino-substituted ligand for the protein of interest N, 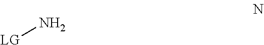
wherein LG is a ligand for the protein of interest,
in the presence of a coupling reagent and an acid/base buffer system;
to afford said fluorescent probe P1 or P2 for the protein of interest.

17. (canceled)

18. (canceled)

19. A method of imaging cannabinoid receptor 2 (CB₂R), comprising contacting said cannabinoid receptor 2 (CB₂R) with a compound of formula (I) according to claim 1.

20. (canceled)
